(19) United States
(12) Patent Application Publication
(10) Pub. No.: US 2025/0154583 A1
(43) Pub. Date: May 15, 2025

(54) METHODS AND NUCLEOTIDE COMPOSITIONS FOR SEQUENCING

(71) Applicant: Singular Genomics Systems, Inc., San Diego, CA (US)

(72) Inventors: Eli N. Glezer, Del Mar, CA (US); Ronald Graham, Carlsbad, CA (US)

(21) Appl. No.: 18/940,449

(22) Filed: Nov. 7, 2024

Related U.S. Application Data
(60) Provisional application No. 63/597,763, filed on Nov. 10, 2023.

(51) Int. Cl. C12Q 1/6874; C12Q 1/6818

(52) U.S. Cl. CPC C12Q 1/6874; C12Q 1/6818

(57) ABSTRACT

Disclosed herein, inter alia, are nucleotide compounds including cleavable moieties and fluorophore moieties, and methods of use thereof.

### CROSS-REFERENCE TO RELATED APPLICATIONS

This application claims the benefit of U.S. Provisional Application No. 63/597,763, filed Nov. 10, 2023, which is incorporated herein by reference in its entirety and for all purposes.

### BACKGROUND

DNA sequencing is a fundamental tool in biological and medical research; it is an essential technology for the paradigm of personalized precision medicine. Among various new DNA sequencing methods, sequencing by synthesis (SBS) is the leading method for realizing the goal of the $1,000 genome. Accordingly, there is a need for modified nucleotides that are effectively recognized as substrates by DNA polymerases and are efficiently and accurately incorporated into growing DNA chains during SBS. Disclosed herein, inter alia, are solutions to these and other problems in the art.

### BRIEF SUMMARY

In an aspect is provided a method of extending a primer, the method including (a) adding an extension solution including four nucleotides to a reaction vessel including a polymerase and the primer, wherein the primer is hybridized to a target polynucleotide, and incorporating one of four nucleotides into the primer, wherein the extension solution includes a first nucleotide including a first fluorophore moiety attached to the nucleotide via a first cleavable linker; a second nucleotide including a second fluorophore moiety attached to the nucleotide via a second cleavable linker; a third nucleotide including a third fluorophore moiety attached to the nucleotide via a third cleavable linker; a fourth nucleotide including a fourth fluorophore moiety attached to the nucleotide via a fourth cleavable linker, wherein the first and the second cleavable linkers are cleavable under identical conditions and the third and the fourth cleavable linkers are cleavable under different identical conditions; (b) exciting a fluorophore, wherein exciting includes: (i) directing a first excitation light and second excitation light at the reaction vessel; (ii) adding a first cleaving agent into the reaction vessel; followed by (iii) adding a second cleaving agent into the reaction vessel.

In an aspect is provided a method of sequencing a template polynucleotide, the method including: (a) contacting a first primer hybridized to the template polynucleotide with four nucleotides and incorporating with a polymerase one of the four nucleotides into the first primer, wherein two of the four nucleotides include a first fluorophore moiety attached to each nucleotide via a first cleavable linker and two of the four nucleotides include a second fluorophore moiety attached to each nucleotide via a second cleavable linker, wherein the first fluorophore moiety generates a first emission light and the second fluorophore moiety generates a second emission light; (b) determining the identity of the incorporated nucleotide by: (i) detecting the first emission light; cleaving the first cleavable linker; and detecting the absence of the first emission light; (ii) detecting the first emission light; cleaving the first cleavable linker; and detecting the first emission light again; (iii) detecting the second emission light; cleaving the first cleavable linker; and detecting the absence of the first emission light; or (iv) detecting the second emission light; cleaving the first cleavable linker; and detecting the second emission light; (c) repeating steps (a) and (b), thereby sequencing a template polynucleotide.

In an aspect is provided a composition including: a first nucleotide including a first fluorophore moiety attached to the nucleotide via a first cleavable linker; a second nucleotide including a second fluorophore moiety attached to the nucleotide via a second cleavable linker; a third nucleotide including a third fluorophore moiety attached to the nucleotide via a third cleavable linker; a fourth nucleotide including a fourth fluorophore moiety attached to the nucleotide via a fourth cleavable linker; wherein the first and the second cleavable linkers are cleavable under identical conditions and the third and the fourth cleavable linkers are cleavable under identical conditions.

### BRIEF DESCRIPTION OF THE DRAWINGS

FIG. 1 illustrates an example of two potential sets of fluorescently labelled nucleotides, where each set differ from the other by the orthogonal cleavable linkers attached between the nucleobase and the fluorophore moiety (shown as the star shape). It is understood that any configuration of the two sets is contemplated herein, for example dATP and dCTP may form the first set, or alternatively dATP and dTTP form the first set. The differences between each set relates to the cleavable linker type. As depicted in FIG. 1, the first set includes an adenine nucleotide covalently labeled with Dye 1 and a guanine nucleotide covalently labeled with Dye 2. The adenine nucleotide and guanine nucleotide are attached to the respective fluorophore moieties via cleavable linker 1. The second set includes a thymine nucleotide covalently labeled with Dye 1 and a cytosine nucleotide covalently labeled with Dye 2. The thymine nucleotide and cytosine nucleotide are attached to the respective fluorophore moieties via cleavable linker 2. The illustrations of the modified nucleotides include a cartoon block on the lower left side of each nucleotide, representing a 3′ reversible terminator. A reversible terminator serves as a temporary stopper for DNA synthesis, allowing the sequencing machinery to determine the identify of the incorporated base before continuing with the next cycle. After the base identification, the reversible terminator is chemically cleaved (e.g., contacting the incorporated nucleotide with a cleaving agent) thereby removing the blocking group. In embodiments, the reversible terminator and the cleavable linker are cleaved under identical conditions. This cleavage reaction allows the DNA synthesis to continue with the next nucleotide incorporation. The reversible nature of the terminator enables repeating this process for multiple cycles, facilitating high-throughput sequencing.

FIG. 2 outlines the dye detection events following successive rounds of imaging and cleaving using the nucleotide sets illustrated in FIG. 1. For example, following incorporation and excitation an image is obtained in both channels and the fluorescence emission signal deriving from Dye 1 of the adenine and the thymine nucleotides as well as the signal from Dye 2 of the guanine and cytosine nucleotides may be detected during the first image event. Though not explicitly shown in the workflow, it is understood that one of the corresponding nucleotides is incorporated into a complementary strand during a sequencing cycle. The possibility of detecting Dye 1 and Dye 2 is depicted by the presence of all the stars in the first row of FIG. 2. After the cleavage of cleavable linker 1, the fluorescence emission signal of Dye 1 from the adenine nucleotide and the fluorescence emission signal of Dye 2 from the guanine nucleotide would be extinguished if either of these nucleotides were incorporated into the complementary strand during the sequencing cycle. Any fluorescence signal detected from Dye 1 or Dye 2 following the cleavage of cleavable linker 1 would derive from the thymine nucleotide and cytosine nucleotide, respectively, as these nucleotides are attached to the respective fluorophore moieties via cleavable linker 2. Following the cleavage of cleavable linker 2, the fluorescence emission signal of Dye 1 or Dye 2 will also be removed and a new sequencing cycle may then occur.

FIG. 3 illustrates the iterative process for base calling using fluorescently labeled nucleotides described herein through iterative steps of excitation of Dye 1 and Dye 2, detection of fluorescence emission signal, cleavage of cleavable linker 1, detection of fluorescence emission signal, and cleavage of cleavable linker 2 from fluorescently labelled nucleotides from FIG. 1. An adenine nucleotide or a guanine nucleotide would be called if the detection of the fluorescence emission from Dye 1 or Dye 2, respectively, was detected prior to the introduction of the cleaving agent for cleavable linker 1 and disappeared following the addition of the cleaving agent. In contrast, a thymine nucleotide and cytosine nucleotide would be called if the detection of the fluorescence emission from Dye 1 or Dye 2, respectively, remained following the introduction of the cleaving agent for cleavable linker 1 but disappeared after the addition of the cleaving agent for cleavable linker 2.

FIGS. 4A-4B. FIG. 4A illustrates an example of two potential sets of fluorescently labelled nucleotides. It is understood that any configuration of the two sets is contemplated herein, for example dATP and dCTP may form the first set, or alternatively dATP and dTTP form the first set. The first set includes an adenine nucleotide covalently labeled with Dye 1 and a guanine nucleotide covalently labeled with Dye 2. The adenine nucleotide and guanine nucleotide are attached to the respective fluorophore moieties via a cleavable linker containing a disulfide moiety. The second set includes a thymine nucleotide covalently labeled with Dye 1 and a cytosine nucleotide covalently labeled with Dye 2. The thymine nucleotide and cytosine nucleotide are attached to the respective fluorophore moieties via a cleavable linker containing vicinal diols. The illustrations of the modified nucleotides include a cartoon block on the lower left side of each nucleotide, representing a 3′ reversible terminator, as described in FIG. 1. FIG. 4B illustrates the iterative process for base calling using fluorescently labeled nucleotides described herein through iterative steps of excitation of Dye 1 and Dye 2, detection of fluorescence emission signal, reductive cleavage of the disulfide moiety present in the first set of nucleotides, detection of fluorescence emission signal, and oxidative cleavage of the vicinal diols present in the second set of nucleotides shown in FIG. 4A. An adenine nucleotide or a guanine nucleotide would be called if the detection of the fluorescence emission from Dye 1 or Dye 2, respectively, was detected prior to the introduction of the cleaving agent for disulfide moiety (e.g., the reducing agent) and disappeared following the addition a reducing agent. In contrast, a thymine nucleotide and cytosine nucleotide would be called if the detection of the fluorescence emission from Dye 1 or Dye 2, respectively, remained following the introduction of the reducing agent.

FIGS. 5A-5B. FIG. 5A illustrates another example of two potential sets of fluorescently labelled nucleotides. It is understood that any configuration of the two sets is contemplated herein, for example dATP and dCTP may form the first set, or alternatively dATP and dTTP form the first set. The first set includes an adenine nucleotide covalently labeled with Dye 1 and a guanine nucleotide covalently labeled with Dye 2. The adenine nucleotide and guanine nucleotide are attached to the respective fluorophore moieties via a cleavable linker containing a disulfide moiety. The second set includes a thymine nucleotide covalently labeled with Dye 1 and a cytosine nucleotide covalently labeled with Dye 2. The thymine nucleotide and cytosine nucleotide are attached to the respective fluorophore moieties via a cleavable linker containing a β-galactosidase substrate moiety. The illustrations of the modified nucleotides include a cartoon block on the lower left side of each nucleotide, representing a 3′ reversible terminator, as described in FIG. 1. The nitro group is depicted with the β-galactosidase substrate moiety as it has been previously shown to facilitate efficient cleavage of the β-galactosidase substrate moiety (see, e.g., Kostova, V. et al. Pharmaceuticals (Basel). 2021 May 7; 14(5):442). FIG. 5B illustrates the iterative process for base calling using fluorescently labeled nucleotides described herein through iterative steps of excitation of Dye 1 and Dye 2, detection of fluorescence emission signal, reductive cleavage of the disulfide moiety present in the first set of nucleotides, detection of fluorescence emission signal, and enzymatic cleavage of the β-galactosidase substrate moiety present in the second set of nucleotides shown in FIG. 5A. An adenine nucleotide or a guanine nucleotide would be called if the detection of the fluorescence emission from Dye 1 or Dye 2, respectively, was detected prior to the introduction of the cleaving agent for disulfide moiety (e.g., the reducing agent) and disappeared following the addition a reducing agent. In contrast, a thymine nucleotide and cytosine nucleotide would be called if the detection of the fluorescence emission from Dye 1 or Dye 2, respectively, remained following the introduction of the reducing agent.

FIG. 6 provides an illustration of the sequential collection of information to inform on the structure of a cell and/or tissue. Spectrally distinct dyes are used in the first set, and optionally reused in subsequent sets. For example, the first set includes Alexa Fluor® 532 (emission: 532 nm), Alexa Fluor® 594 (emission: 594 nm), Alexa Fluor® 647 (emission: 647 nm), and Alexa Fluor® 680 (emission: 680 nm) to illuminate the Golgi Apparatus, endoplasmic reticulum, actin, lysosomes, and specific cell surface receptors of a cell. Following cleavage and removal of the fluorophores, the second set of targeting molecules are incubated with the sample cell. The second set can then illuminate the nucleus, nucleoli, mitochondria, nuclear envelop, cell surface receptors, and plasma membrane. The sequential addition of cell paints can continue for N cycles providing additional information about the cell. The resulting images may be computationally processed and overlaid to provide a composite image of the cell and/or tissue.

### DETAILED DESCRIPTION

The aspects and embodiments described herein relate to modified nucleotides including cleavable linkers, reversible terminators, and fluorophore moieties and methods of use thereof in 2-color sequencing.

### I. Definitions

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by,
″\*MERGEFORMAT\*MERGEFORMAT —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to:
″\*MERGEFORMAT\*MERGEFORMAT —CH₂—CH₂—O—CH₃, —CH₂—CH₂—NH—CH₃, —CH₂—CH₂—N(CH₃)—CH₃, —CH₂—S—CH₂—CH₃,
″\*MERGEFORMAT\*MERGEFORMAT —CH₂—S—CH₂, —S(O)—CH₃, —CH₂—CH₂—S(O)₂—CH₃, —CH═CHO—CH₃, —Si(CH₃)₃,
″\*MERGEFORMAT\*MERGEFORMAT —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as
″\*MERGEFORMAT\*MERGEFORMAT —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, =N—OR′, —NR′R″, —SR′, -halogen, —SiR′R″R″′, —OC(O)R′,
″\*MERGEFORMAT\*MERGEFORMAT —C(O)R′, —CO₂R′, —CONR′R″, —OC(O)NR′R″, —NR″C(O)R′, —NR′—C(O)NR″R″′, —NR″C(O)₂R′,
″\*MERGEFORMAT\*MERGEFORMAT —NR—C(NR′R″R″′)═NR″″, —NR—C(NR′R″)═NR″′, —S(O)R′, —S(O)₂R′, —S(O)₂NR′R″, —NRSO₂R′, —NR′NR″R″′, —ONR′R″, —NR′C(O)NR″NR′″R″″, —CN, —NO₂, —NR′SO₂R″, —NR′C(O)R″,
″\*MERGEFORMAT\*MERGEFORMAT —NR′C(O)—OR″, —NR′OR″, in a number ranging from zero to (2m′+1), where m′ is the total number of carbon atoms in such radical. R, R′, R″, R″′, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R″′, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen,
″\*MERGEFORMAT\*MERGEFORMAT —SiR′R″R″, —OC(O)R′, —C(O)R′, —CO₂R′, —CONR′R″, —OC(O)NR′R″, —NR″C(O)R′,
″\*MERGEFORMAT\*MERGEFORMAT —NR′—C(O)NR″R″′, —NR″C(O)₂R′, —NR—C(NR′R″R″′)=NR″″, —NR—C(NR′R″)═NR″′, —S(O)R′,
″\*MERGEFORMAT\*MERGEFORMAT —S(O)₂R′, —S(O)₂NR′R″, —NRSO₂R′, —NR′NR″R″′, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″,
″\*MERGEFORMAT\*MERGEFORMAT —NR′C(O)—OR″, —NR′OR″, in a number ranging from zero to the total number of open valences on the aromatic ring system; and where R′, R″, R″′, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R″′, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —PO₃H, —PO₄H, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —PO₃H, —PO₄H, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —PO₃H, —PO₄H, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₁₂, —OCHBr₂, —OCHI₂, —OCHF₂, —PO₃H, —PO₄H, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound (e.g., nucleotide analogue) is a chemical species set forth in the Examples section, claims, embodiments, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Where a moiety is substituted (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene), the moiety is substituted with at least one substituent (e.g., a substituent group, a size-limited substituent group, or lower substituent group) and each substituent is optionally different. Additionally, where multiple substituents are present on a moiety, each substituent may be optionally different.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

“Analog,” “analogue” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹⁰ substituents are present, each R¹⁰ substituent may be distinguished as R^{10.1}, R^{10.2}, R^{10.3}, R^{10.4}, etc., wherein each of R^{10.1}, R^{10.2}, R^{10.3}, R^{10.4}, etc. is defined within the scope of the definition of R¹⁰ and optionally differently. Where an R moiety, group, or substituent as disclosed herein is attached through the representation of a single bond and the R moiety, group, or substituent is oxo, a person having ordinary skill in the art will immediately recognize that the oxo is attached through a double bond in accordance with the normal rules of chemical valency.

Descriptions of the compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The compounds of the present invention may exist as salts, such as with pharmaceutically acceptable acids. The present invention includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art. The neutral forms of the compounds are preferably regenerated by contacting the salt with a base or acid and isolating the parent compound in the conventional manner. The parent form of the compound may differ from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

“Contacting” is used in accordance with its plain ordinary meaning and refers to the process of allowing at least two distinct species (e.g., chemical compounds including biomolecules or cells, or bioconjugate reactive moieties) to become sufficiently proximal to react, interact or physically touch. It should be appreciated, however, that the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound as described herein and a nucleotide, linker, protein, or enzyme.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Nucleic acid,” “oligonucleotide,” “nucleic acid molecule,” “nucleic acid sequence,” “nucleic acid fragment,” or “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. The term “nucleic acid” includes single- or double-stranded DNA, RNA and analogs (derivatives) thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. Oligonucleotides are typically from about 5, 6, 7, 8, 9, 10, 12, 15, 25, 30, 40, 50 or more nucleotides in length, up to about 100 nucleotides in length. Nucleic acids and polynucleotides are polymers of any length, including longer lengths, e.g., 200, 300, 500, 1000, 2000, 3000, 5000, 7000, 10,000, etc. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support. In certain embodiments the nucleic acids herein contain phosphodiester bonds. In other embodiments, nucleic acid analogs are included that may have alternate backbones, comprising, e.g., phosphoramidate, phosphorothioate, phosphorodithioate, or O-methylphosphoroamidite linkages (see, Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. A residue of a nucleic acid, as referred to herein, is a monomer of the nucleic acid (e.g., a nucleotide).

“Nucleotide,” as used herein, refers to a nucleoside-5′-polyphosphate compound, or a structural analog thereof, which can be incorporated (e.g., partially incorporated as a nucleoside-5′-monophosphate or derivative thereof) by a nucleic acid polymerase to extend a growing nucleic acid chain (such as a primer). Nucleotides may include bases such as guanine (G), adenine (A), thymine, (T), uracil (U), cytosine (C), or analogues thereof, and may comprise 2, 3, 4, 5, 6, 7, 8, or more phosphates in the phosphate group. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleotides may be modified at one or more of the base, sugar, or phosphate group. A nucleotide may have a label or tag attached (a “labeled nucleotide” or “tagged nucleotide”). In embodiments, the nucleotide is a modified nucleotide which terminates primer extension reversibly. In embodiments, nucleotides may further include a polymerase-compatible cleavable moiety covalently bound to the 3′ oxygen.

A “nucleoside” is structurally similar to a nucleotide but lacks the phosphate moieties. The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine and inosine. Nucleosides may be modified at the base and/or the sugar. An example of a nucleoside analog would be one in which the label is linked to the base and there is no phosphate group attached to the sugar molecule.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/ or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g., phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

In embodiments, “nucleotide analogue,” “nucleotide analog,” or “nucleotide derivative” shall mean an analogue of A, G, C, T or U (that is, an analogue or derivative of a nucleotide comprising the base A, G, C, T or U), including a phosphate group, which may be recognized by DNA or RNA polymerase (whichever is applicable) and may be incorporated into a strand of DNA or RNA (whichever is appropriate). Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the —OH group at the 3′-position of deoxyribose. Nucleotide analogues and DNA polymerase-based DNA sequencing are also described in U.S. Pat. No. 6,664,079, which is incorporated herein by reference in its entirety for all purposes.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

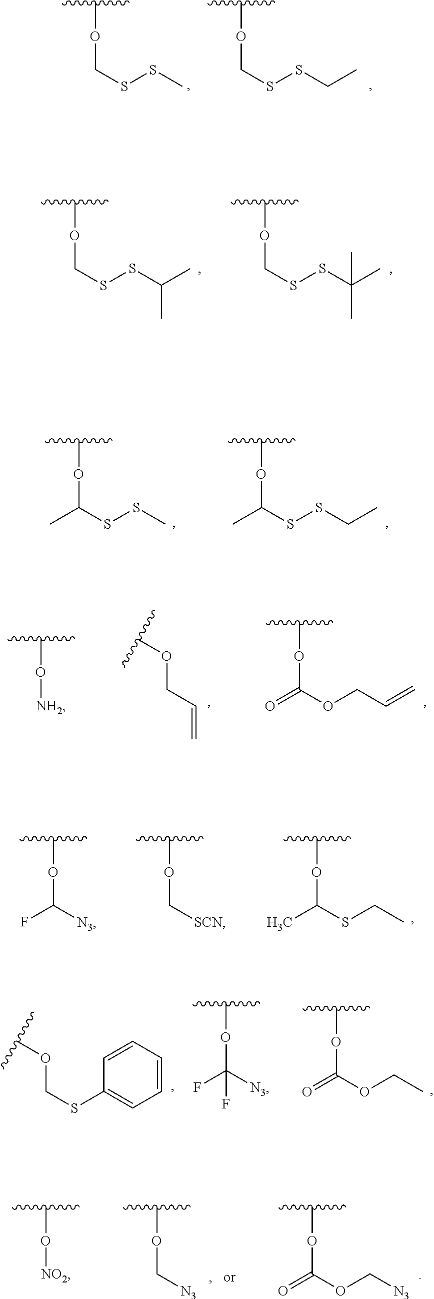

A label moiety of a modified nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the OH group at the 3′-position of deoxyribose. Nucleotide analogues and DNA polymerase-based DNA sequencing are also described in U.S. Pat. No. 6,664,079, which is incorporated herein by reference in its entirety for all purposes. Non-limiting examples of detectable labels include labels including fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the dye is a fluorescent dye. Non-limiting examples of dyes, some of which are commercially available, include CF® dyes (Biotium, Inc.), Atto™ dyes (ATTO-TEC GmbH), Alexa Fluor® dyes (Thermo Fisher), DyLight® dyes (Thermo Fisher), Cy® dyes (GE Healthscience), IRDye® dyes (Li-Cor Biosciences, Inc.), and HiLyte™ dyes (Anaspec, Inc.). In embodiments, the label is a fluorophore.

In some embodiments, a nucleic acid includes a label. As used herein, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF® dyes (Biotium, Inc.), Atto™ dyes (ATTO-TEC GmbH), Alexa Fluor® dyes (Thermo Fisher), DyLight® dyes (Thermo Fisher), Cy® dyes (GE Healthscience), IRDye® dyes (Li-Cor Biosciences, Inc.), and HiLyte™ dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing). Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa Fluor® dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy3®). In embodiments, the cyanine moiety has 5 methine structures (i.e., cyanine 5 or Cy5®). In embodiments, the cyanine moiety has 7 methine structures (i.e., cyanine 7 or Cy7®).

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. In general, the conditions under which a removable group is removed are compatible with a process employing the removable group (e.g., an amplification process or sequencing process).

As used herein, the terms “reversible blocking groups” and “reversible terminators” are used in accordance with their plain and ordinary meanings and refer to a blocking moiety located, for example, at the 3′ position of a modified nucleotide and may be a chemically cleavable moiety such as an allyl group, an azidomethyl group or a methoxymethyl group, or may be an enzymatically cleavable group such as a phosphate ester. Non-limiting examples of nucleotide blocking moieties are described in applications WO 2004/018497, WO 96/07669, U.S. Pat. Nos. 7,057,026, 7,541,444, 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is attached to the 3′-oxygen of the nucleotide, having the formula

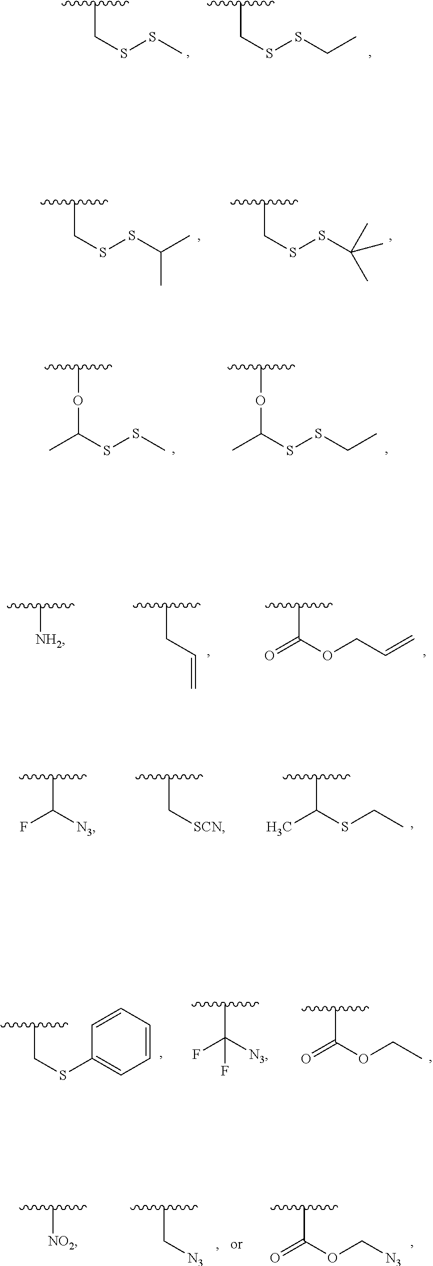

wherein the 3′ oxygen of the nucleotide is not shown in the formulae above. The term “allyl” as described herein refers to an unsubstituted methylene attached to a vinyl group (i.e., —CH═CH₂). In embodiments, the reversible terminator moiety is

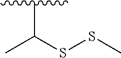 [SMILES: *C(C)SSC]

as described in U.S. Pat. No. 10,738,072 or 11,174,281, each of which are incorporated herein by reference for all purposes. For example, a nucleotide including a reversible terminator moiety may be represented by the formula:

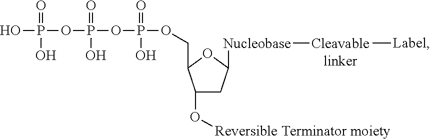

where the nucleobase is adenine or adenine analogue, thymine or thymine analogue, guanine or guanine analogue, or cytosine or cytosine analogue.

In some embodiments, a nucleic acid (e.g., a probe or a primer) includes a molecular identifier or a molecular barcode. As used herein, the term “molecular barcode” (which may be referred to as a “tag”, a “barcode”, a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads including the barcode can be identified as originating from a single sample polynucleotide molecule on the basis of the barcode alone. In other embodiments, individual barcode sequences may be used more than once, but adapters including the duplicate barcodes are associated with different sequences and/or in different combinations of barcoded adaptors, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random. In some embodiments, a barcode may include a nucleic acid sequence from within a pool of known sequences. In some embodiments, the barcodes may be pre-defined. In embodiments, the barcodes are selected to form a known set of barcodes, e.g., the set of barcodes may be distinguished by a particular Hamming distance. In embodiments, each barcode sequence is unique within the known set of barcodes. In embodiments, each barcode sequence is associated with a particular oligonucleotide.

In embodiments, a nucleic acid (e.g., an adapter or primer) includes a sample barcode. In general, a “sample barcode” is a nucleotide sequence that is sufficiently different from other sample barcode to allow the identification of the sample source based on sample barcode sequence(s) with which they are associated. In embodiments, a plurality of nucleotides (e.g., all nucleotides from a particular sample source, or sub-sample thereof) are joined to a first sample barcode, while a different plurality of nucleotides (e.g., all nucleotides from a different sample source, or different subsample) are joined to a second sample barcode, thereby associating each plurality of polynucleotides with a different sample barcode indicative of sample source. In embodiments, each sample barcode in a plurality of sample barcodes differs from every other sample barcode in the plurality by at least three nucleotide positions, such as at least 3, 4, 5, 6, 7, 8, 9, 10, or more nucleotide positions. In some embodiments, substantially degenerate sample barcodes may be known as random. In some embodiments, a sample barcode may include a nucleic acid sequence from within a pool of known sequences. In some embodiments, the sample barcodes may be pre-defined. In embodiments, the sample barcode includes about 1 to about 10 nucleotides. In embodiments, the sample barcode includes about 3, 4, 5, 6, 7, 8, 9, or about 10 nucleotides. In embodiments, the sample barcode includes about 3 nucleotides. In embodiments, the sample barcode includes about 5 nucleotides. In embodiments, the sample barcode includes about 7 nucleotides. In embodiments, the sample barcode includes about 10 nucleotides. In embodiments, the sample barcode includes about 6 to about 10 nucleotides.

The term “complement” is used in accordance with its plain and ordinary meaning and refers to a nucleotide (e.g., RNA nucleotide or DNA nucleotide) or a sequence of nucleotides capable of base pairing with a complementary nucleotide or sequence of nucleotides (e.g., Watson-Crick base pairing). As described herein and commonly known in the art the complementary (matching) nucleotide of adenosine is thymidine and the complementary (matching) nucleotide of guanosine is cytosine. Thus, a complement may include a sequence of nucleotides that base paired with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. Another example of complementary sequences are a template sequence and an amplicon sequence polymerized by a polymerase along the template sequence. “Duplex” means at least two oligonucleotides and/or polynucleotides that are fully or partially complementary undergo Watson-Crick type base pairing among all or most of their nucleotides so that a stable complex is formed. Complementary single stranded nucleic acids and/or substantially complementary single stranded nucleic acids can hybridize to each other under hybridization conditions, thereby forming a nucleic acid that is partially or fully double stranded. When referring to a double-stranded polynucleotide including a first strand hybridized to a second strand, it is understood that each of the first strand and the second strand are independently single-stranded polynucleotides. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, an oligonucleotide is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides, made up of “dNTPs,” which do not have a hydroxyl group at the 2′ position, and oligoribonucleotides, made up of “NTPs,” which have a hydroxyl group in the 2′ position. Oligonucleotides also may include derivatives, in which the hydrogen of the hydroxyl group is replaced with an organic group, e.g., an allyl group.

Oligonucleotides, as described herein, typically are capable of forming hydrogen bonds with oligonucleotides having a complementary base sequence. These bases may include the natural bases, such as A, G, C, T, and U, as well as artificial, non-standard or non-natural nucleotides such as iso-cytosine and iso-guanine. As described herein, a first sequence of an oligonucleotide is described as being 100% complementary with a second sequence of an oligonucleotide when the consecutive bases of the first sequence (read 5′-to-3′) follow the Watson-Crick rule of base pairing as compared to the consecutive bases of the second sequence (read 3′-to-5′). An oligonucleotide may include nucleotide substitutions. For example, an artificial base may be used in place of a natural base such that the artificial base exhibits a specific interaction that is similar to the natural base.

As used herein, the terms “polynucleotide primer” and “primer” refers to any polynucleotide molecule that may hybridize to a polynucleotide template, be bound by a polymerase, and be extended in a template-directed process for nucleic acid synthesis (e.g., amplification and/or sequencing). The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In certain embodiments, a primer has a length of 10 to 150 nucleotides, 15 to 150 nucleotides, 5 to 100 nucleotides, 5 to 50 nucleotides or 10 to 50 nucleotides. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “primer binding sequence” refers to a polynucleotide sequence that is complementary to at least a portion of a primer (e.g., a sequencing primer or an amplification primer). Primer binding sequences can be of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amio acid on a protein or polypeptide through a covalent, non-covalent or other interaction.

As used herein, a “platform primer” is a primer oligonucleotide immobilized or otherwise bound to a solid support (i.e. an immobilized oligonucleotide). Examples of platform primers include P7 and P5 primers (i.e., Illumina® platform sequences), or S1 and S2 primers (i.e., Singular Genomics® platform sequences), or the reverse complements thereof. A “platform primer binding sequence” refers to a sequence or portion of an oligonucleotide that is capable of binding to a platform primer (e.g., the platform primer binding sequence is complementary to the platform primer). In embodiments, a platform primer binding sequence may form part of an adapter. In embodiments, a platform primer binding sequence is complementary to a platform primer sequence. In embodiments, a platform primer binding sequence is complementary to a primer. Illumina is a registered trademark of Illumina, Inc. Singular Genomics is a registered trademark of Singular Genomics Systems, Inc.

The order of elements within a nucleic acid molecule is typically described herein from 5′ to 3′. In the case of a double-stranded molecule, the “top” strand is typically shown from 5′ to 3′, according to convention, and the order of elements is described herein with reference to the top strand.

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9° N polymerase or a variant thereof, E. coli DNA polymerase I, Bacteriophage T4 DNA polymerase, Sequenase™ (GE Healthcare), Taq DNA polymerase, DNA polymerase from Bacillus stearothermophilus, Bst 2.0 DNA polymerase, 9° N polymerase (exo-)A485L/Y409V, Phi29 DNA Polymerase ((φ29 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or or Therminator™ IX DNA Polymerase. In embodiments, the polymerase is a protein polymerase. Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g. Therminator™ γ, 9° N polymerase (exo-), Therminator™ II, Therminator™ III, or Therminator™ IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884. For example, a polymerase catalyzes the addition of a next correct nucleotide to the 3′-OH group of the primer via a phosphodiester bond, thereby chemically incorporating the nucleotide into the primer. Optionally, the polymerase used in the provided methods is a processive polymerase. Optionally, the polymerase used in the provided methods is a distributive polymerase. Therminator™ is a trademark of New England Biolabs.

As used herein, the term “incorporating” or “chemically incorporating,” when used in reference to a primer and cognate nucleotide, refers to the process of joining the cognate nucleotide to the primer or extension product thereof by formation of a phosphodiester bond.

As used herein, the term “selective” or “selectivity” or the like of a compound refers to the compound's ability to discriminate between molecular targets. For example, a chemical reagent may selectively modify one nucleotide type in that it reacts with one nucleotide type (e.g., cytosines) and not other nucleotide types (e.g., adenine, thymine, or guanine). When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the term “template polynucleotide” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In embodiments, the template polynucleotide includes a target nucleic acid sequence and one or more barcode sequences. In embodiments, the template polynucleotide is a barcode sequence.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances, two or more associated species are “tethered”, “coated”, “attached”, or “immobilized” to one another or to a common solid or semisolid support (e.g. a receiving substrate). An association may refer to a relationship, or connection, between two entities. For example, a barcode sequence may be associated with a particular target by binding a probe including the barcode sequence to the target. In embodiments, detecting the associated barcode provides detection of the target. Associated may refer to the relationship between a sample and the DNA molecules, RNA molecules, or polynucleotides originating from or derived from that sample. These relationships may be encoded in oligonucleotide barcodes, as described herein. A polynucleotide is associated with a sample if it is an endogenous polynucleotide, i.e., it occurs in the sample at the time the sample is obtained, or is derived from an endogenous polynucleotide. For example, the RNAs endogenous to a cell are associated with that cell. cDNAs resulting from reverse transcription of these RNAs, and DNA amplicons resulting from PCR amplification of the cDNAs, contain the sequences of the RNAs and are also associated with the cell. The polynucleotides associated with a sample need not be located or synthesized in the sample, and are considered associated with the sample even after the sample has been destroyed (for example, after a cell has been lysed). Barcoding can be used to determine which polynucleotides in a mixture are associated with a particular sample. In embodiments, a proximity probe is associated with a particular barcode, such that identifying the barcode identifies the probe with which it is associated. Because the proximity probe specifically binds to a target, identifying the barcode thus identifies the target.

The term “adapter” as used herein refers to any oligonucleotide that can be ligated to a nucleic acid molecule, thereby generating nucleic acid products that can be sequenced on a sequencing platform (e.g., an Illumina® or Singular Genomics G4® sequencing platform). In embodiments, adapters include two reverse complementary oligonucleotides forming a double-stranded structure. In embodiments, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In embodiments, adapters include sequences that bind to sequencing primers. In embodiments, adapters include sequences that bind to immobilized oligonucleotides (e.g., P7 and P5 sequences) or reverse complements thereof. In embodiments, the adapter is substantially non-complementary to the 3′ end or the 5′ end of any target polynucleotide present in the sample. In embodiments, the adapter can include a sequence that is substantially identical, or substantially complementary, to at least a portion of a primer, for example a universal primer. In embodiments, the adapter can include an index sequence (also referred to as barcode or tag) to assist with downstream error correction, identification or sequencing. In some embodiments, an adapter is hairpin adapter (also referred to herein as a hairpin). In some embodiments, a hairpin adapter includes a single nucleic acid strand including a stem-loop structure. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter. In some embodiments, a method herein includes ligating a first adapter to a first end of a double stranded nucleic acid, and ligating a second adapter to a second end of a double stranded nucleic acid. In some embodiments, the first adapter and the second adapter are different. For example, in certain embodiments, the first adapter and the second adapter may include different nucleic acid sequences or different structures. In some embodiments, the first adapter is a Y-adapter and the second adapter is a hairpin adapter. In some embodiments, the first adapter is a hairpin adapter and a second adapter is a hairpin adapter. In certain embodiments, the first adapter and the second adapter may include different primer binding sites, different structures, and/or different capture sequences (e.g., a sequence complementary to a capture nucleic acid). In some embodiments, some, all or substantially all of the nucleic acid sequence of a first adapter and a second adapter are the same. In some embodiments, some, all or substantially all of the nucleic acid sequence of a first adapter and a second adapter are substantially different.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

The term “bioconjugate group” or “bioconjugate reactive moiety” or “bioconjugate reactive group” refers to a chemical moiety which participates in a reaction to form bioconjugate linker (e.g., covalent linker). Non-limiting examples of bioconjugate reactive groups and the resulting bioconjugate reactive linkers may be found in the Bioconjugate Table below:

| Bioconjugate | Bioconjugate | |
|---|---|---|
| reactive group 1 | reactive group 2 | |
| (e.g., electrophilic | (e.g., nucleophilic | Resulting |
| bioconjugate | bioconjugate | Bioconjugate |
| reactive moiety) | reactive moiety) | reactive linker |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

As used herein, the term “bioconjugate” or “bioconjugate linker” refers to the resulting association between atoms or molecules of bioconjugate reactive groups. The association can be direct or indirect. For example, a conjugate between a first bioconjugate reactive group (e.g.,
″\*MERGEFORMAT\*MERGEFORMAT —NH₂, —COOH, —N— hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g.,
″\*MERGEFORMAT\*MERGEFORMAT -sulfo-N-hydroxysuccinimide moiety) is covalently attached to the second bioconjugate reactive group (e.g., an amine).

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc. (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (l) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate includes a molecular entity derived from the reaction of an unsaturated bond, such as a maleimide, and a sulfhydryl group.

In embodiments, the compounds of the present disclosure use a cleavable linker to attach a label (e.g., a fluorophore moiety) to the molecule. The use of the term “cleavable linker” is not meant to imply that the whole linker is required to be removed. The cleavage site can be located at a position on the linker that ensures that part of the linker remains attached to the molecule after cleavage.

The term “cleavable linker” or “cleavable moiety” refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄)). In embodiments, a chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase.

A photocleavable linker (e.g., including or consisting of an o-nitrobenzyl group) refers to a linker which is capable of being split in response to photo-irradiation (e.g., ultraviolet radiation). An acid-cleavable linker refers to a linker which is capable of being split in response to a change in the pH (e.g., increased acidity). A base-cleavable linker refers to a linker which is capable of being split in response to a change in the pH (e.g., decreased acidity). An oxidant-cleavable linker refers to a linker which is capable of being split in response to the presence of an oxidizing agent. A reductant-cleavable linker refers to a linker which is capable of being split in response to the presence of an reducing agent (e.g., Tris(3-hydroxypropyl)phosphine). In embodiments, the cleavable linker is a dialkylketal linker, an azo linker, an allyl linker, a cyanoethyl linker, a 1-(4,4-dimethyl-2,6-dioxocyclohex-1-ylidene)ethyl linker, or a nitrobenzyl linker.

The term “orthogonally cleavable linker” or “orthogonal cleavable linker” as used herein refers to a cleavable linker that is cleaved by a first cleaving agent (e.g., enzyme, nucleophilic/basic reagent, reducing agent, photo-irradiation, electrophilic/acidic reagent, organometallic and metal reagent, oxidizing reagent) in a mixture of two or more different cleaving agents and is not cleaved by any other different cleaving agent in the mixture of two or more cleaving agents. For example, two different cleavable linkers are both orthogonal cleavable linkers when a mixture of the two different cleavable linkers are reacted with two different cleaving agents and each cleavable linker is cleaved by only one of the cleaving agents and not the other cleaving agent. In embodiments, an orthogonally is a cleavable linker that following cleavage the two separated entities (e.g., fluorescent dye, bioconjugate reactive group) do not further react and form a new orthogonally cleavable linker. In embodiments, each of the two nucleotides of a first nucleotide set include a first orthogonal cleavable linker. For example, each nucleotide is attached to a fluorophore moiety via a cleavable linker capable of being cleaved by a reducing agent. It follows that, in embodiments, the second nucleotide set includes two nucleotides each including a fluorophore moiety attached via a second orthogonal cleavable linker that does not cleave upon contact with a reducing agent that cleaved the first orthogonal cleavable linker. For example, the second orthogonal cleavable linker is an acid-cleavable linker (i.e., cleavable upon contact with an acid).

The term “orthogonal detectable label” or “orthogonal detectable moiety” as used herein refer to a detectable label (e.g., fluorescent dye or detectable dye) that is capable of being detected and identified (e.g., by use of a detection means (e.g., emission wavelength, physical characteristic measurement)) in a mixture or a panel (collection of separate samples) of two or more different detectable labels. For example, two different detectable labels that are fluorescent dyes are both orthogonal detectable labels when a panel of the two different fluorescent dyes is subjected to a wavelength of light that is absorbed by one fluorescent dye but not the other and results in emission of light from the fluorescent dye that absorbed the light but not the other fluorescent dye. Orthogonal detectable labels may be separately identified by different absorbance or emission intensities of the orthogonal detectable labels compared to each other and not only be the absolute presence of absence of a signal. An example of a set of four orthogonal detectable labels is the set of Rox™-Labeled Tetrazine, Alexa Fluor® 488-Labeled SHA, Cy5®-Labeled Streptavidin, and R6G-Labeled Dibenzocyclooctyne.

The term “salt” refers to acid or base salts of the compounds described herein. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. In embodiments, compounds may be presented with a positive charge, for example

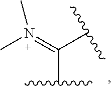

and it is understood an appropriate counter-ion (e.g., chloride ion, fluoride ion, or acetate ion) may also be present, though not explicitly shown. Likewise, for compounds having a negative charge

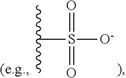 [SMILES: *S(=O)(=O)[O-]]

it is understood an appropriate counter-ion (e.g., a proton, sodium ion, potassium ion, or ammonium ion) may also be present, though not explicitly shown. The protonation state of the compound (e.g., a compound described herein) depends on the local environment (i.e., the pH of the environment), therefore, in embodiments, the compound may be described as having a moiety in a protonated state

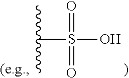 [SMILES: *S(=O)(=O)O]

or an ionic state

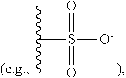 [SMILES: *S(=O)(=O)[O-]]

and it is understood these are interchangeable. In embodiments, the counter-ion is represented by the symbol M (e.g., M⁺ or M⁻).

The term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The term “allyl” as described herein refers to an unsubstituted methylene attached to a vinyl group (i.e., —CH═CH₂), having the formula

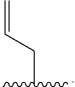 [SMILES: *CC=C]

An “allyl linker” refers to a divalent unsubstituted methylene attached to a vinyl group, having the formula

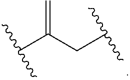 [SMILES: *CC(*)=C]

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or
″\*MERGEFORMAT\*MERGEFORMAT —CH₃). Likewise, for a linker variable (e.g., L¹, L², L³, or L⁴ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

As used herein, the terms “specific”, “specifically”, “specificity”, or the like of a compound refers to the compound's ability to cause a particular action, such as binding, to a particular molecular target with minimal or no action to other proteins in the cell.

The terms “attached,” “bind,” and “bound” as used herein are used in accordance with their plain and ordinary meanings and refer to an association between atoms or molecules. The association can be direct or indirect. For example, attached molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

“Specific binding” is where the binding is selective between two molecules. A particular example of specific binding is that which occurs between an antibody and an antigen. Typically, specific binding can be distinguished from non-specific when the dissociation constant (KD) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. In embodiments, the KD (equilibrium dissociation constant) between two specific binding molecules is less than 10⁻⁶ M, less than 10⁻⁷ M, less than 10⁻⁸ M, less than 10⁻⁹ M, less than 10⁻⁹ M, less than 10⁻¹¹ M, or less than about 10⁻¹² M or less.

As used herein, a “specific binding reagent” or “specific binding agent” refers to an agent that binds specifically to a particular biomolecule (e.g., carbohydrate, cell surface receptor, protein, nucleic acid, or lipid molecule). Examples of a specific binding reagent include, but are not limited to, an antibody or target-specific oligonucleotide.

As used herein, the term “proximity probe” or “probe” (e.g., a first probe or a second probe described herein) is used in accordance with its plain ordinary meaning and refers to a specific binding agent (e.g., an antibody) attached to an oligonucleotide. In embodiments, sets of probes can be employed to target multiple biomolecules of interest. Alternatively, in embodiments, a plurality of probes may be employed for a single biomolecule of interest.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein includes contacting a template and an annealed primer with a suitable polymerase under conditions suitable for polymerase extension and/or sequencing. The sequencing methods are preferably carried out with the target polynucleotide arrayed on a solid substrate. Multiple target polynucleotides can be immobilized on the solid support through linker molecules, or can be attached to particles, e.g., microspheres, which can also be attached to a solid substrate. In embodiments, the solid substrate is in the form of a chip, a bead, a well, a capillary tube, a slide, a wafer, a filter, a fiber, a porous media, or a column. In embodiments, the solid substrate is gold, quartz, silica, plastic, glass, diamond, silver, metal, or polypropylene. In embodiments, the solid substrate is porous.

The term “particle” means a small body made of a rigid or semi-rigid material. The body can have a shape characterized, for example, as a sphere, oval, microsphere, or other recognized particle shape whether having regular or irregular dimensions. The particles may in one way or another rest upon a two dimensional surface by magnetic, gravitational, or ionic forces, or by chemical bonding, or by any other means known to those skilled in the art. In further embodiments, the bead may have magnetic properties. Further the beads may have a density that allows them to rest upon a two dimensional surface in solution. Particles may consist of glass, polystyrene, latex, metal, quantum dot, polymers, silica, metal oxides, ceramics, or any other substance suitable for binding to nucleic acids, or chemicals or proteins which can then attach to nucleic acids. The particles may be rod shaped or spherical or disc shaped, or comprise any other shape. The particles may also be distinguishable by their shape or size or physical location. The particles may be distinguished through spectroscopy by having a composition containing dyes or fluorochromes in various ratios or concentrations. The particles may also be distinguishable by barcode or holographic images or other imprinted forms of particle coding. Where the particles are magnetic particles, they may be attracted to the surface of the chamber by application of a magnetic field and the magnetic particles may be dispersed from the surface of the chamber by removal of the magnetic field. The magnetic particles are preferably paramagnetic or superparamagnetic.

The term “gel” in this context refers to a semi-rigid solid that is permeable to liquids and gases. As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers. Hydrogels can contain over 99% water and may comprise natural or synthetic polymers, or a combination thereof. Hydrogels also possess a degree of flexibility very similar to natural tissue, due to their significant water content. A detailed description of suitable hydrogels may be found in published U.S. patent application 2010/0055733, herein specifically incorporated by reference. By “hydrogel subunits” or “hydrogel precursors” is meant hydrophilic monomers, prepolymers, or polymers that can be crosslinked, or “polymerized”, to form a three-dimensional (3D) hydrogel network. Hydrogels can be derived from a single species of monomer or from two or more different monomer species with at least one hydrophilic component. Hydrogels may be prepared by cross-linking hydrophilic biopolymers or synthetic polymers. Thus, in some embodiments, the hydrogel may include a crosslinker. As used herein, the term “crosslinker” refers to a molecule that can form a three-dimensional network when reacted with the appropriate base monomers. Examples of the hydrogel polymers, which may include one or more crosslinkers, include but are not limited to, hyaluronans, chitosans, agar, heparin, sulfate, cellulose, alginates (including alginate sulfate), collagen, dextrans (including dextran sulfate), pectin, carrageenan, polylysine, gelatins (including gelatin type A), agarose, (meth)acrylate-oligolactide-PEO-oligolactide-(meth)acrylate, PEO-PPO-PEO copolymers (Pluronics), poly(phosphazene), poly(methacrylates), poly(N-vinylpyrrolidone), PL(G)A-PEO-PL(G)A copolymers, poly(ethylene imine), polyethylene glycol (PEG)-thiol, PEG-acrylate, acrylamide, N,N′-bis(acryloyl)cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetracrylate, or combinations thereof. Thus, for example, a combination may include a polymer and a crosslinker, for example polyethylene glycol (PEG)-thiol/PEG-acrylate, acrylamide/N,N′-bis(acryloyl)cystamine (BACy), or PEG/polypropylene oxide (PPO). In embodiments, the hydrogel includes chemical crosslinks (e.g., intermolecular or intramolecular joining of two or more molecules by a covalent bond) and may be referred to as a chemical hydrogel. In embodiments, the hydrogel includes physical crosslinks (e.g., intermolecular or intramolecular joining of two or more molecules by a non-covalent bond) and may be referred to as a physical hydrogel. In embodiments, the physical hydrogel include one or more crosslinks including hydrogen bonds, hydrophobic interactions, and/or polymer chain entanglements.

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer.

The term “polymer” refers to a molecule including repeating subunits (e.g., polymerized monomers). For example, polymeric molecules may be based upon polyethylene glycol (PEG), tetraethylene glycol (TEG), polyvinylpyrrolidone (PVP), poly(xylene), or poly(p-xylylene). The term “polymerizable monomer” is used in accordance with its meaning in the art of polymer chemistry and refers to a compound that may covalently bind chemically to other monomer molecules (such as other polymerizable monomers that are the same or different) to form a polymer. In embodiments, polymer refers to PEG, having the formula:

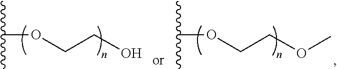

wherein n is an integer from 1 to 30.

Polymers can be hydrophilic, hydrophobic or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers.

As used herein, the term “substrate” refers to a solid support material. The substrate can be non-porous or porous. The substrate can be rigid or flexible. As used herein, the terms “solid support” and “solid surface” refers to discrete solid or semi-solid surface. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A nonporous substrate generally provides a seal against bulk flow of liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor® (Zeon Corporation), silica or silica-based materials including silicon and modified silicon, carbon, metals, inorganic glasses, optical fiber bundles, photopatternable dry film resists, UV-cured adhesives and polymers. Particularly useful solid supports for some embodiments have at least one surface located within a flow cell. Solid surfaces can also be varied in their shape depending on the application in a method described herein. For example, a solid surface useful herein can be planar, or contain regions which are concave or convex. In embodiments, the geometry of the concave or convex regions (e.g., wells) of the solid surface conform to the size and shape of the particle to maximize the contact between as substantially circular particle. In embodiments, the wells of an array are randomly located such that nearest neighbor features have random spacing between each other. Alternatively, in embodiments the spacing between the wells can be ordered, for example, forming a regular pattern. The term solid substrate is encompassing of a substrate (e.g., a flow cell) having a surface including a polymer coating covalently attached thereto. In embodiments, the solid substrate is a flow cell. The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip), for example a metal surface (e.g., steel, gold, silver, aluminum, silicon and copper). In embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In embodiments a substrate includes a magnetic material (e.g., iron, nickel, cobalt, platinum, aluminum, and the like). In embodiments a substrate includes a magnetic bead (e.g., DYNABEADS™ (Invitrogen), hematite, AMPure XP). Magnets can be used to purify and/or capture nucleic acids bound to certain substrates (e.g., substrates including a metal or magnetic material). The flow cell is typically a glass slide containing small fluidic channels (e.g., a glass slide 75 mm×25 mm×1 mm having one or more channels), through which sequencing solutions (e.g., polymerases, nucleotides, and buffers) may traverse. Though typically glass, suitable flow cell materials may include polymeric materials, plastics, silicon, quartz (fused silica), Borofloat® (SCHOTT) glass, silica, silica-based materials, carbon, metals, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength. Conversely, it may be desirable to select a material that does not pass radiation of a certain wavelength (e.g., being opaque, absorptive, or reflective). In embodiments, the material of the flow cell is selected due to the ability to conduct thermal energy. In embodiments, a flow cell includes inlet and outlet ports and a flow channel extending there between.

As used herein, the term “channel” refers to a passage in or on a substrate material that directs the flow of a fluid. A channel may run along the surface of a substrate, or may run through the substrate between openings in the substrate. A channel can have a cross section that is partially or fully surrounded by substrate material (e.g., a fluid impermeable substrate material). For example, a partially surrounded cross section can be a groove, trough, furrow or gutter that inhibits lateral flow of a fluid. The transverse cross section of an open channel can be, for example, U-shaped, V-shaped, curved, angular, polygonal, or hyperbolic. A channel can have a fully surrounded cross section such as a tunnel, tube, or pipe. A fully surrounded channel can have a rounded, circular, elliptical, square, rectangular, or polygonal cross section. In particular embodiments, a channel can be located in a flow cell, for example, being embedded within the flow cell. A channel in a flow cell can include one or more windows that are transparent to light in a particular region of the wavelength spectrum. In embodiments, the channel contains one or more polymers of the disclosure. In embodiments, the channel is filled by the one or more polymers, and flow through the channel (e.g., as in a sample fluid) is directed through the polymer in the channel. In embodiments, the tissue is in a channel of a flow cell.

As used herein, the term “inlet” or “inlet port” refers to the location on a flow cell assembly where the reagents and fluids used for methods described herein enters the flow cell. As used herein, the term “outlet” or “outlet port” refers to the location on a flow cell assembly where the reagents and fluids used for methods described herein exits the flow cell after contacting the reaction chamber containing the cell or tissue to be analyzed.

As used herein, the term “reaction chamber” refers to a contained space or vessel designed for conducting chemical, biological, or physical reactions. A reaction chamber may include features such as inlets and outlets for introducing and removing substances, sensors for monitoring reaction conditions, and mechanisms for agitation or mixing. In embodiments, the reaction chamber is a part of the flow cell where the cell or tissue is in contact with the fluids (e.g., buffers), polymerases, nucleotides, and reagents used for the methods described herein. In embodiments, the reaction chamber is an enclosed (i.e., closed) container containing one or two openings for introducing and removing fluids and reagents.

The term “surface” is intended to mean an external part or external layer of a substrate. The surface can be in contact with another material such as a gas, liquid, gel, polymer, organic polymer, second surface of a similar or different material, metal, or coat. The surface, or regions thereof, can be substantially flat. The substrate and/or the surface can have surface features such as wells, pits, channels, ridges, raised regions, pegs, posts or the like.

The term “microplate”, or “multiwell container” as used herein, refers to a substrate including a surface, the surface including a plurality of reaction chambers separated from each other by interstitial regions on the surface. In embodiments, the microplate has dimensions as provided and described by American National Standards Institute (ANSI) and Society for Laboratory Automation And Screening (SLAS); for example the tolerances and dimensions set forth in ANSI SLAS 1-2004 (R2012); ANSI SLAS 2-2004 (R2012); ANSI SLAS 3-2004 (R2012); ANSI SLAS 4-2004 (R2012); and ANSI SLAS 6-2012, which are incorporated herein by reference. The dimensions of the microplate as described herein and the arrangement of the reaction chambers may be compatible with an established format for automated laboratory equipment. In embodiments, the device described herein provides methods for high-throughput screening. High-throughput screening (HTS) refers to a process that uses a combination of modern robotics, data processing and control software, liquid handling devices, and/or sensitive detectors, to efficiently process a large amount of (e.g., thousands, hundreds of thousands, or millions) samples in biochemical, genetic, or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 μl, 200 μl, 100 μl, 50 μl or less. Through this process, one can rapidly identify active compounds, small molecules, antibodies, proteins or polynucleotides in a cell.

The reaction chambers may be provided as wells of a multiwell container (alternatively referred to as reaction chambers), for example a microplate may contain 2, 4, 6, 12, 24, 48, 96, 384, or 1536 sample wells. In embodiments, the 96 and 384 wells are arranged in a 2:3 rectangular matrix. In embodiments, the 24 wells are arranged in a 3:8 rectangular matrix. In embodiments, the 48 wells are arranged in a 3:4 rectangular matrix. In embodiments, the reaction chamber is a microscope slide (e.g., a glass slide about 75 mm by about 25 mm). In embodiments the slide is a concavity slide (e.g., the slide includes a depression). In embodiments, the slide includes a coating for enhanced cell adhesion (e.g., poly-L-lysine, silanes, carbon nanotubes, polymers, epoxy resins, or gold). In embodiments, the microplate is about 5 inches by about 3.33 inches, and includes a plurality of 5 mm diameter wells. In embodiments, the microplate is about 5 inches by about 3.33 inches and includes a plurality of 6 mm diameter wells. In embodiments, the microplate is about 5 inches by about 3.33 inches and includes a plurality of 7 mm diameter wells. In embodiments, the microplate is about 5 inches by about 3.33 inches and includes a plurality of 7.5 mm diameter wells. In embodiments, the microplate is 5 inches by 3.33 inches, and includes a plurality of 7.5 mm diameter wells. In embodiments, the microplate is about 5 inches by about 3.33 inches and includes a plurality of 8 mm diameter wells. In embodiments, the microplate is a flat glass or plastic tray in which an array of wells are formed, wherein each well can hold between from a few microliters to hundreds of microliters of fluid reagents and samples. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 5-7 mm. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 6 mm.

The term “well” refers to a discrete concave feature in a substrate having a surface opening that is completely surrounded by interstitial region(s) of the surface. Wells can have any of a variety of shapes at their opening in a surface including but not limited to round, elliptical, square, polygonal, or star shaped (i.e., star shaped with any number of vertices). The cross section of a well taken orthogonally with the surface may be curved, square, polygonal, hyperbolic, conical, or angular. The wells of a microplate are available in different shapes, for example F-Bottom: flat bottom; C-Bottom: bottom with minimal rounded edges; V-Bottom: V-shaped bottom; or U-Bottom: U-shaped bottom. In embodiments, the well is substantially square. In embodiments, the well is square. In embodiments, the well is F-bottom. In embodiments, the microplate includes 24 substantially round flat bottom wells. In embodiments, the microplate includes 48 substantially round flat bottom wells. In embodiments, the microplate includes 96 substantially round flat bottom wells. In embodiments, the microplate includes 384 substantially square flat bottom wells.

The discrete regions (i.e., features, wells) of the microplate may have defined locations in a regular array, which may correspond to a rectilinear pattern, circular pattern, hexagonal pattern, or the like. In embodiments, the pattern of wells includes concentric circles of regions, spiral patterns, rectilinear patterns, hexagonal patterns, and the like. In embodiments, the pattern of wells is arranged in a rectilinear or hexagonal pattern A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. In embodiments, interstitial regions have a surface material that differs from the surface material of the wells (e.g., the interstitial region contains a photoresist and the surface of the well is glass). In embodiments, interstitial regions have a surface material that is the same as the surface material of the wells (e.g., both the surface of the interstitial region and the surface of well contain a polymer or copolymer).

As used herein, the term “sequencing reaction mixture” is used in accordance with its plain and ordinary meaning and refers to an aqueous mixture that contains the reagents necessary to allow dNTP or dNTP analogue (e.g., a modified nucleotide) to add a nucleotide to a DNA strand by a DNA polymerase. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

As used herein, the term “sequencing cycle” is used in accordance with its plain and ordinary meaning and refers to incorporating one or more nucleotides (e.g., nucleotide analogues) to the 3′ end of a polynucleotide with a polymerase and detecting one or more labels useful for identifying the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions.

As used herein, the term “base calling” refers to the determination the identity of the nucleobase (A, G, C, or T) that was incorporated for each cluster on a flow cell during a sequencing cycle. Images at the clusters on the flow cell are acquired to facilitate the determination of the identity of the nucleobase (A, G, C, or T), and the base calling is based on the measurement of the fluorescence emission signal using an emission filter specific for the maximum fluorescence emission wavelength and detection of absence of the detected fluorescence emission signal following the introduction of orthogonal cleaving agents specific to one of the two orthogonal cleavable linkers.

As used herein, the term “extension” or “elongation” is used in accordance with their plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In embodiments, a sequencing read includes reading a barcode sequence and a template nucleotide sequence. In embodiments, a sequencing read includes reading a template nucleotide sequence. In embodiments, a sequencing read includes reading a barcode and not a template nucleotide sequence. Reads of length 20-40 base pairs (bp) are referred to as ultra-short. Typical sequencers produce read lengths in the range of 100-500 bp. Read length is a factor which can affect the results of biological studies. For example, longer read lengths improve the resolution of de novo genome assembly and detection of structural variants. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. In some embodiments, a sequencing read may include 300, 400, 500, 600, 700, 800, 900, 1,000, 1,100, 1,200, 1,300, 1,400, 1,500, or more nucleotide bases.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic. As used herein, the term “multiplex” is used to refer to an assay in which multiple (i.e. at least two) different biomolecules are assayed at the same time, and more particularly in the same aliquot of the sample, or in the same reaction mixture. In embodiments, more than two different biomolecules are assayed at the same time. In embodiments, at least 2, 4, 6, 8, 10, 20, 50, 100, 200, 300, 400, 500, 600, 700, 800, 900, 1000, 1100, 1200, 1300, 1400 or 1500 or more biomolecules are detected according to the present method.

Complementary single stranded nucleic acids and/or substantially complementary single stranded nucleic acids can hybridize to each other under hybridization conditions, thereby forming a nucleic acid that is partially or fully double stranded. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

“Hybridize” shall mean the annealing of a nucleic acid sequence to another nucleic acid sequence (e.g., one single-stranded nucleic acid (such as a primer) to another nucleic acid) based on the well-understood principle of sequence complementarity. In an embodiment the other nucleic acid is a single-stranded nucleic acid. In some embodiments, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution.

As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acids. For example, specific hybridization includes the hybridization of a primer or capture nucleic acid to a portion of a target nucleic acid (e.g., a template, or adapter portion of a template) that is substantially complementary to the primer or capture nucleic acid. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double stranded portion of nucleic acid.

As used herein, the term “adjacent,” refers to two nucleotide sequences in a nucleic acid, can refer to nucleotide sequences separated by 0 to about 20 nucleotides, more specifically, in a range of about 1 to about 10 nucleotides, or to sequences that directly abut one another. As those of skill in the art appreciate, two nucleotide sequences that that are to ligated together will generally directly abut one another.

A nucleic acid can be amplified by a suitable method. The term “amplification,” “amplified” or “amplifying” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof (which may be referred to herein as an “amplification product” or “amplification products”). In some embodiments an amplification reaction comprises a suitable thermal stable polymerase. Thermal stable polymerases are known and are stable for prolonged periods of time, at temperature greater than 80° C. when compared to common polymerases found in most mammals. In certain embodiments the term “amplification,” “amplified” or “amplifying” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

As used herein, bridge-PCR (bPCR) amplification is a method for solid-phase amplification as exemplified by the disclosures of U.S. Pat. Nos. 5,641,658; 7,115,400; and U.S. Patent Publ. No. 2008/0009420, each of which is incorporated herein by reference in its entirety. Bridge-PCR involves repeated polymerase chain reaction cycles, cycling between denaturation, annealing, and extension conditions and enables controlled, spatially-localized, amplification, to generate amplification products (e.g., amplicons) immobilized on a solid support in order to form arrays comprised of colonies (or “clusters”) of immobilized nucleic acid molecule.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA (oligonucleotide ligation assay)/PCR, PCR/OLA, LDR/PCR, PCR/PCR/LDR, PCR/LDR, LCR/PCR, PCR/LCR (also known as combined chain reaction—CCR), and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. Nos. 6,027,998; 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27: e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May; 53(2):165-74, Schweitzer et al., Curr Opin Biotechnol. 2001 February; 12(1):21-7, U.S. Pat. Nos. 5,830,711, 6,027,889, 5,686,243, PCT Publication No. WO0056927A3, and PCT Publication No. WO9803673A1.

In some embodiments, amplification includes at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can include thermocycling or can be performed isothermally.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in-vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments solid phase amplification includes a nucleic acid amplification reaction including only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge PCR amplification, emulsion PCR, WildFire amplification (e.g., US patent publication US20130012399), the like or combinations thereof.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example, an array can have at least about 100 features/cm², at least about 1,000 features/cm², at least about 10,000 features/cm², at least about 100,000 features /cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

Provided herein are methods, systems, and compositions for analyzing a sample (e.g., sequencing nucleic acids within a sample) in situ. The term “in situ” is used in accordance with its ordinary meaning in the art and refers to a sample surrounded by at least a portion of its native environment, such as may preserve the relative position of two or more elements. For example, an extracted human cell obtained is considered in situ when the cell is retained in its local microenvironment so as to avoid extracting the target (e.g., nucleic acid molecules or proteins) away from their native environment. An in situ sample (e.g., a cell) can be obtained from a suitable subject. An in situ cell sample may refer to a cell and its surrounding milieu, or a tissue. A sample can be isolated or obtained directly from a subject or part thereof. In embodiments, the methods described herein (e.g., sequencing a plurality of target nucleic acids of a cell in situ) are applied to an isolated cell (i.e., a cell not surrounded by least a portion of its native environment). For the avoidance of any doubt, when the method is performed within a cell (e.g., an isolated cell) the method may be considered in situ. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). A sample may include a cell and RNA transcripts. A sample can include nucleic acids obtained from one or more subjects. In some embodiments a sample includes nucleic acid obtained from a single subject. A subject can be any living or non-living organism, including but not limited to a human, non-human animal, plant, bacterium, fungus, virus, or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a plant. In some embodiments, a subject is a human subject. A subject can be a patient (e.g., a human patient). In some embodiments a subject is suspected of having a genetic variation or a disease or condition associated with a genetic variation.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. A protein may refer to a protein expressed in a cell.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods, such as a population of prokaryotic or eukaryotic organisms, including bacteria or yeast.

The term “cellular component” is used in accordance with its ordinary meaning in the art and refers to any organelle, nucleic acid, protein, or analyte that is found in a prokaryotic, eukaryotic, archaeal, or other organismic cell type. Examples of cellular components (e.g., a component of a cell) include RNA transcripts, proteins, membranes, lipids, and other analytes.

A “gene” refers to a polynucleotide that is capable of conferring biological function after being transcribed and/or translated. Functionally, a genome is subdivided into genes. Each gene is a nucleic acid sequence that encodes an RNA or polypeptide. A gene is transcribed from DNA into RNA, which can either be non-coding (ncRNA) with a direct function, or an intermediate messenger (mRNA) that is then translated into protein. Typically a gene includes multiple sequence elements, such as for example, a coding element (i.e., a sequence that encodes a functional protein), non-coding element, and regulatory element. Each element may be as short as a few bp to 5kb. In embodiments, the gene is the protein coding sequence of RNA. Non-limiting examples of genes include developmental genes (e.g., adhesion molecules, cyclin kinase inhibitors, Wnt family members, Pax family members, Winged helix family members, Hox family members, cytokines/lymphokines and their receptors, growth/differentiation factors and their receptors, neurotransmitters and their receptors); oncogenes (e.g., ABL1, BCL1, BCL2, BCL6, CBFA2, CBL, CSF1R, ERBA, ERBB, ERBB2, ETS1, ETS1, ETV6, FGR, FOS, FYN, HCR, HRAS, JUN, KRAS, LCK, LYN, MDM2, MLL, MYB, MYC, MYCL1, MYCN, NRAS, PIM1, PML, RET, SRC, TAL1, TCL3, and YES); tumor suppressor genes (e.g., APC, BRCA1, BRCA2, MADH4, MCC, NF1, NF2, RB1, TP53, and WTI1); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases). In embodiments, a gene includes at least one mutation associated with a disease or condition mediated by a mutant form of the gene.

As used herein, “biomaterial” refers to any biological material produced by an organism. In some embodiments, biomaterial includes secretions, extracellular matrix, proteins, lipids, organelles, membranes, cells, portions thereof, and combinations thereof. In some embodiments, cellular material includes secretions, extracellular matrix, proteins, lipids, organelles, membranes, cells, portions thereof, and combinations thereof. In some embodiments, biomaterial includes viruses. In some embodiments, the biomaterial is a replicating virus and thus includes virus infected cells. In embodiments, a biological sample includes biomaterials.

As used herein, the terms “biomolecule”, “analyte”, or “target molecule” refers to an agent (e.g., a compound, macromolecule, or small molecule), and the like derived from a biological system (e.g., an organism, a cell, or a tissue) to be detected using the methods and compositions described herein. The biomolecule (i.e., target molecule) may contain multiple individual components that collectively construct the biomolecule, for example, in embodiments, the biomolecule (i.e., target molecule) is a polynucleotide wherein the polynucleotide is composed of nucleotide monomers. The biomolecule (i.e., target molecule) may be or may include DNA, RNA, organelles, carbohydrates, lipids, proteins, or any combination thereof. These components may be extracellular. In some examples, the biomolecule (i.e., target molecule) may be referred to as a clump or aggregate of combinations of components. In some instances, the biomolecule (i.e., target molecule) may include one or more constituents of a cell but may not include other constituents of the cell. In embodiments, a biomolecule (i.e., target molecule) is a molecule produced by a biological system (e.g., an organism). The biomolecule (i.e., target molecule) may be any substance (e.g., molecule) or entity that is desired to be detected by the method of the invention. The biomolecule is the “target” of the assay method of the invention. The biomolecule (i.e., target molecule) may accordingly be any compound that may be desired to be detected, for example a peptide or protein, or nucleic acid molecule or a small molecule, including organic and inorganic molecules. The biomolecule (i.e., target molecule) may be a cell or a microorganism, including a virus, or a fragment or product thereof. Biomolecules of particular interest (i.e., target molecules) may thus include proteinaceous molecules such as peptides, polypeptides, proteins or prions or any molecule which includes a protein or polypeptide component, etc., or fragments thereof. The biomolecule (i.e., target molecule) may be a single molecule or a complex that contains two or more molecular subunits, which may or may not be covalently bound to one another, and which may be the same or different. Thus, in addition to cells or microorganisms, such a complex biomolecule (i.e., target molecule) may also be a protein complex. Such a complex may thus be a homo- or hetero-multimer. Aggregates of molecules e.g., proteins may also be target analytes, for example aggregates of the same protein or different proteins. The biomolecule (i.e., target molecule) may also be a complex between proteins or peptides and nucleic acid molecules such as DNA or RNA. Of particular interest may be the interactions between proteins and nucleic acids, e.g., regulatory factors, such as transcription factors, and interactions between DNA or RNA molecules.

The term “organelle” as used herein refers to an entity of cell associated with a particular function. In embodiments, an organelle refers to a specialized subunit within a cell that has a specific function, and is usually separately enclosed within its own lipid bilayer. Examples of organelles include the nucleus, mitochondria, endoplasmic reticulum, Golgi apparatus, lysosomes, and chloroplasts (in plant cells). Although most organelles are functional units within cells, some organelles function extend outside of cells, such as cilia, flagellum, archaellum, and the trichocyst. In embodiments, the organelle is a membrane bound organelle. In embodiments, the organelle is a non-membrane bound organelle. Non-membrane bounded organelles, also called biomolecular complexes, are assemblies of macromolecules such as the ribosome, the spliceosome, the proteasome, the nucleosome, and the centriole. Commonly detected organelles includes the nucleus, which is often visualized using dyes such as DAPI, Hoechst, and SYTO™ Green, mitochondria are with MitoTracker™ dyes and Rhodamine 123, endoplasmic reticulum (ER) utilizing dyes like ER-Tracker® Green/Red or DiOC6, the Golgi apparatus is stained with BODIPY™ FL C5-Ceramide and NBD C6-Ceramide, lysosomes are typically stained using LysoTracker™ dyes and Acridine Orange, and peroxisomes may be stained with Peroxisome-Tracker® Red and Peroxy Green dyes. Although not membrane-bound, ribosomes may detected using antibodies such as anti-RPL10 or anti-RPS6. Additionally, the cytoskeleton, specifically actin filaments, is frequently stained to study cell shape with Phalloidin conjugates and Alexa Fluor® Phalloidin being widely used. In embodiments, the organelle is a biomolecular complex including a plurality of subunits. In embodiments, the organelle is a macromolecule. In embodiments, the organelle is a eukaryotic organelle. In embodiments, the organelle is the cell membrane, the endoplasmic reticulum, a flagellum, a Golgi apparatus, a mitochondria, the nucleus, a vacuole. In embodiments, the organelle is a lysosome. In embodiments, the organelle is the nucleolus.

In some embodiments, a sample includes one or more nucleic acids, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

The methods and kits of the present disclosure may be applied, mutatis mutandis, to the sequencing of RNA, or to determining the identity of a ribonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits. Vessels may include any structure capable of supporting or containing a liquid or solid material and may include, tubes, vials, jars, containers, tips, etc. In embodiments, a wall of a vessel may permit the transmission of light through the wall. In embodiments, the vessel may be optically clear. The kit may include the enzyme and/or nucleotides in a buffer.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

In some embodiments, a “sample” includes one or more nucleic acids, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid.

The term “covalent linker” is used in accordance with its ordinary meaning and refers to a divalent moiety which connects at least two moieties to form a molecule.

The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

As used herein, the term “upstream” refers to a region in the nucleic acid sequence that is towards the 5′ end of a particular reference point, and the term “downstream” refers to a region in the nucleic acid sequence that is toward the 3′ end of the reference point.

As used herein, the terms “incubate,” and “incubation refer collectively to altering the temperature of an object in a controlled manner such that conditions are sufficient for conducting the desired reaction. Thus, it is envisioned that the terms encompass heating a receptacle (e.g., a microplate) to a desired temperature and maintaining such temperature for a fixed time interval. Also included in the terms is the act of subjecting a receptacle to one or more heating and cooling cycles (i.e., “temperature cycling” or “thermal cycling”). While temperature cycling typically occurs at relatively high rates of change in temperature, the term is not limited thereto, and may encompass any rate of change in temperature.

The term “isolated” means altered or removed from the natural state. For example, a nucleic acid or a polypeptide naturally present in a living animal is not isolated, but the same nucleic acid or polypeptide partially or completely separated from the coexisting materials of its natural state is isolated. An isolated nucleic acid or protein can exist in substantially purified form, or can exist in a non-native environment such as, for example, a host cell. In embodiments, “isolated” refers to a nucleic acid, polynucleotide, polypeptide, protein, or other component that is partially or completely separated from components with which it is normally associated (other proteins, nucleic acids, cells, etc.).

The term “synthetic target” as used herein refers to a modified protein or nucleic acid such as those constructed by synthetic methods. In embodiments, a synthetic target is artificial or engineered, or derived from or contains an artificial or engineered protein or nucleic acid (e.g., non-natural or not wild type). For example, a polynucleotide that is inserted or removed such that it is not associated with nucleotide sequences that normally flank the polynucleotide as it is found in nature is a synthetic target polynucleotide.

The term “nucleic acid sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics® (e.g., the G4® system), Illumina™ (e.g., HiSeq™, MiSeq™, NextSeq™, or NovaSeq™ systems), Life Technologies™ (e.g., ABI PRISM™, or SOLiD™ systems), Pacific Biosciences (e.g., systems using SMRT™ Technology such as the Sequel™ or RS II™ systems), or Qiagen (e.g., Genereader™ system). Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), ascorbic acid, tris(hydroxymethyl)aminomethane or “Tris”), aqueous salts (e.g., KCl or (NH4)2SO4)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA). Non-limited examples of reservoirs include cartridges, pouches, vials, containers, and eppendorf tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light emitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the illuminator or light source is a light-emitting diode (LED). The LED can be, for example, an Organic Light Emitting Diode (OLED), a Thin Film Electroluminescent Device (TFELD), or a Quantum dot based inorganic organic LED. The LED can include a phosphorescent OLED (PHOLED). In embodiments, the nucleic acid sequencing device includes an imaging system (e.g., an imaging system as described herein). The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a system described herein and may include a fluorescence spectrophotometer including an objective lens and/or a solid-state imaging device. The solid-state imaging device may include a charge coupled device (CCD) and/or a complementary metal oxide semiconductor (CMOS). The system may also include circuitry and processors, including systems using microcontrollers, reduced instruction set computers (RISC), application specific integrated circuits (ASICs), field programmable gate array (FPGAs), logic circuits, and any other circuit or processor capable of executing functions described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. In embodiments, the device includes a thermal control assembly useful to control the temperature of the reagents.

The term “image” is used according to its ordinary meaning and refers to a representation of all or part of an object. The representation may be an optically detected reproduction. For example, an image can be obtained from fluorescent, luminescent, scatter, or absorption signals. The part of the object that is present in an image can be the surface or other xy plane of the object. Typically, an image is a 2 dimensional representation of a 3 dimensional object. An image may include signals at differing intensities (i.e., signal levels). An image can be provided in a computer readable format or medium. An image is derived from the collection of focus points of light rays coming from an object (e.g., the sample), which may be detected by any image sensor.

The term “xy coordinates” refers to information that specifies location, size, shape, and/or orientation in an xy plane. The information can be, for example, numerical coordinates in a Cartesian system. The coordinates can be provided relative to one or both of the x and y axes or can be provided relative to another location in the xy plane (e.g., a fiducial). The term “xy plane” refers to a 2 dimensional area defined by straight line axes x and y. When used in reference to a detecting apparatus and an object observed by the detector, the xy plane may be specified as being orthogonal to the direction of observation between the detector and object being detected.

As used herein, the term “tissue section” refers to a piece of tissue that has been obtained from a subject, optionally fixed and attached to a surface, e.g., a microscope slide.

As used herein, the term “code,” means a system of rules to convert information, such as signals obtained from a detection apparatus, into another form or representation, such as a base call or nucleic acid sequence. For example, signals that are produced by one or more incorporated nucleotides can be encoded by a digit. The digit can have several potential values, each value encoding a different signal state. For example, a binary digit will have a first value for a first signal state and a second value for a second signal state. A digit can have a higher radix including, for example, a ternary digit having three potential values, a quaternary digit having four potential values, etc. A series of digits can form a codeword. The length of the codeword is the same as the number of sequencing steps performed. Exemplary codes include, but are not limited to, a Hamming code. A Hamming code is used in accordance with its ordinary meaning in computer science, mathematics, telecommunication sciences and refers to a code that can be used to detect and correct the errors that can occur when the data is moved or stored. The Hamming distance refers to the difference in integer number between two codewords of equal length, and may be determined using known techniques in the art such as the Hamming distance test or the Hamming distance algorithm. For example, for two codewords (i.e., two sequenced barcodes that have been converted to a string of integers), a difference of 0 indicates that the codewords (i.e., the sequences) are identical. A difference of 1 in integer value indicates a Hamming distance of 1, thus 1 base difference between the oligos. Hamming distance is the number of positions for which the corresponding bit values in the two strings are different. In other words, the test measures the minimum number of substitutions that would be necessary to change one bit string into the other.

As used herein, the term “identification oligonucleotide” can also refer to a “barcode” or “index” or “unique molecular identifier (UMI)” and refers to a known nucleic acid sequence which has feature(s) that can be identified. Typically, an identification oligonucleotide is unique to a particular feature in a pool of identification oligonucleotide that differ from one another in sequence, and each of which is associated with a different feature. In embodiments, identification oligonucleotides are about or at least about 5, 6, 7, 8, 9, 10, 15, 20, 25, 30, 40, 50, 75 or more nucleotides in length. In embodiments, identification oligonucleotides are shorter than 20, 15, 10, 9, 8, 7, 6, or 5 nucleotides in length. In embodiments, identification oligonucleotides are 10-50 nucleotides in length, such as 15-40 or 20-30 nucleotides in length. In a pool of different identification oligonucleotides, identification oligonucleotides may have the same or different lengths. In general, identification oligonucleotides are of sufficient length and include sequences that are sufficiently different to allow the identification of associated features (e.g., a binding agent or analyte) based on identification oligonucleotides with which they are associated. In embodiments, an identification oligonucleotide can be identified accurately after the mutation, insertion, or deletion of one or more nucleotides in the identification oligonucleotide sequence, such as the mutation, insertion, or deletion of 1, 2, 3, 4, 5, or more nucleotides. In embodiments, each identification oligonucleotide in a plurality of identification oligonucleotides differs from every other identification oligonucleotide in the plurality by at least three nucleotide positions, such as at least 3, 4, 5, 6, 7, 8, 9, 10, or more nucleotide positions.

The terms “detect” and “detecting” as used herein refer to the act of viewing (e.g., imaging, indicating the presence of, quantifying, or measuring (e.g., spectroscopic measurement), an agent based on an identifiable characteristic of the agent, for example, the light emitted from the present compounds. For example, the compound described herein can be bound to an agent, and, upon being exposed to an absorption light, will emit an emission light. The presence of an emission light can indicate the presence of the agent. Likewise, the quantification of the emitted light intensity can be used to measure the concentration of the agent.

The term “detectable moiety” or “detectable agent” or “detectable label” can also refer to a “label” or “labels” and generally refer to molecules that can directly or indirectly produce or result in a detectable signal either by themselves or upon interaction with another molecule. Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” In embodiments, “detectable moiety” or “detectable agent” or “detectable label” refers to a compound containing a fluorescent dye moiety or derivatives thereof, which can be used to detect a target analyte or biomolecule of interest. Detection of a detectable label is typically accomplished by measuring an emission wavelength emitted by the fluorescent dye moiety following its absorption of an excitation light at a specific wavelength. In embodiments, a detectable label is conjugated to a biomolecule through a covalent linker. In embodiments, a detectable label is conjugated to a biomolecule through a cleavable linker. Examples of detectable moieties include fluorescein, rhodamine, acridine dyes, Alexa Fluor® dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e. cyanine 3 or Cy3®). In embodiments, the cyanine moiety has 5 methine structures (i.e. cyanine 5 or Cy5®). In embodiments, the cyanine moiety has 7 methine structures (i.e., cyanine 7 or Cy7®). In embodiments, a detectable moiety is a moiety (e.g., monovalent form) of a detectable agent.

The terms “fluorophore,” “fluorescent agent,” “fluorescent dye,” or “fluorescent dye moiety” are used interchangeably and refer to a substance, compound, agent, or composition (e.g., compound) that can absorb light at one or more wavelengths and re-emit light at one or more longer wavelengths, relative to the one or more wavelengths of absorbed light. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Examples of fluorophores that may be included in the compounds and compositions described herein include fluorescent proteins, xanthene derivatives (e.g., fluorescein, rhodamine, Oregon green, eosin, or Texas red), cyanine and derivatives (e.g., cyanine, indocarbocyanine, oxacarbocyanine, thiacarbocyanine, or merocyanine), napththalene derivatives (e.g., dansyl or prodan derivatives), coumarin and derivatives, oxadiazole derivatives (e.g., pyridyloxazole, nitrobenzoxadiazole or benzoxadiazole), anthracene derivatives (e.g., anthraquinones, DRAQ5™, DRAQ7™, or CyTrak Orange™), pyrene derivatives (e.g., Cascade Blue® and derivatives), oxazine derivatives (e.g., Nile red, Nile blue, cresyl violet, or oxazine 170), acridine derivatives (e.g., proflavin, acridine orange, acridine yellow), arylmethine derivatives (e.g., auramine, crystal violet, or malachite green), tetrapyrrole derivatives (e.g., porphin, phthalocyanine, bilirubin), CF dye™, DRAQ™, CyTRAK™, BODIPY®, ATTO™ dyes (ATTO-TEC GmbH), Alexa Fluor™, DyLight™ Fluor™, Atto™, Tracy™, FluoProbes™ Abberior Dyes™, DY™ dyes, MegaStokes Dyes™, Sulfo Cy™, Seta™ dyes, SeTau™ dyes, Square Dyes™, Quasar™ dyes, Cal Fluor™ dyes, SureLight Dyes™, PerCP™ Phycobilisomes™, APC™, APCXL™, RPE™, and/or BPE™. A fluorescent moiety is a radical of a fluorescent agent. The emission from the fluorophores can be detected by any number of methods, including but not limited to, fluorescence spectroscopy, fluorescence microscopy, fluorimeters, fluorescent plate readers, infrared scanner analysis, laser scanning confocal microscopy, automated confocal nanoscanning, laser spectrophotometers, fluorescent-activated cell sorters (FACS), image-based analyzers and fluorescent scanners (e.g., gel/membrane scanners).

The term “directing an excitation light on a reaction vessel” refers to irradiating a sample including a fluorophore with a light including sufficient amplitude and wavelength to promote absorption and subsequent emission of the fluorophore. A light source (e.g., a laser, LED (light emitting diode), a mercury or tungsten lamp, or a super-continuous diode) can provide electromagnetic radiation in the ultraviolet (UV) range (about 200 to 390 nm), visible (VIS) range (about 390 to 770 nm). This light is then selectively filtered to a specific wavelength or a band of wavelengths (e.g., an excitation wavelength) to illuminate a sample containing a biomolecule. (See Lord, S. J., et al., Anal Chem. 2010 Mar. 15; 82(6): 2192-2203). In embodiments, the excitation light directed onto a reaction vessel has a wavelength between 200 nm to 1500 nm. In embodiments, the excitation beam directed onto a reaction vessel has a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm.

As used herein, the term “fluorescently labelled nucleotide” is used in accordance with its ordinary meaning in the art and refers to a nucleotide that is covalently attached to a fluorophore moiety via a covalent linker (e.g., covalently attached to the nucleobase). Commercially available examples of fluorescently labelled nucleotides include, but are not limited to, fluorescein-12-dCTP and rhodamine-12-dCTP (Jena Bioscience).

The term “detecting an emission light” is used in accordance with its ordinary meaning in the art and refers to the process of capturing and optionally quantifying light emitted from a fluorescent compound using a detector (e.g., charge-coupled device (CCD), avalanche photodiodes, or photomultiplier tubes (PMTs)). In embodiments, detecting a light emission includes detecting light with a wavelength of 400-800 nm. In embodiments, detecting a light emission includes detecting light with a wavelength of 443 nm, 506 nm, 512 nm, 514 nm, 517 nm, 518 nm, 519 nm, 520 nm, 521 nm, 523 nm, 526 nm, 527 nm, 533 nm, 537 nm, 540 nm, 548 nm, 550 nm, 554 nm, 555 nm, 556 nm, 565 nm, 568 nm, 572 nm, 573 nm, 574 nm, 575 nm, 578 nm, 580 nm, 590 nm, 591 nm, 595 nm, 596 nm, 603 nm, 605 nm, 615 nm, 617 nm, 618 nm, 619 nm, 630 nm, 647 nm, 650 nm, 665 nm, 670 nm, 690 nm, 694 nm, 702 nm, 723 nm, or 775 nm. In embodiments, detecting a light emission includes detecting light with a wavelength in the near-infrared spectrum. In embodiments, detecting a light emission includes detecting light with a maximum emission wavelength from 600 nm-900 nm. In embodiments, detecting a light emission includes detecting light with a maximum emission wavelength from 600 nm-1450 nm. In embodiments, detecting a light emission includes detecting light with a maximum emission wavelength from 1000 nm-1700 nm. In embodiments, detecting a light emission includes detecting light with a maximum emission wavelength in the “imaging window,” which refers to a range of wavelengths where tissue autofluorescence is minimal and the absorption and emission of light in tissue results in minimal light scattering (see, e.g., Pansare et al. Chem Mater. 2012 Mar. 13; 24(5): 812-827 and Wang et al. ACS Cent Sci. 2020 Aug. 26; 6(8): 1302-1316).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### II. Compositions & Kits

In an aspect is provided a composition including four nucleotides, wherein each nucleotide includes a different nucleobase (e.g., adenine, guanine, thymine, and cytosine). In embodiments, the composition includes a first nucleotide including a first fluorophore moiety attached to the nucleotide via a first cleavable linker; a second nucleotide including a second fluorophore moiety attached to the nucleotide via a second cleavable linker; a third nucleotide including a third fluorophore moiety attached to the nucleotide via a third cleavable linker; a fourth nucleotide including a fourth fluorophore moiety attached to the nucleotide via a fourth cleavable linker. In embodiments, the first and the second cleavable linkers each include a first orthogonal cleavable linker, and the third and the fourth cleavable linkers each include a second orthogonal cleavable linker, wherein the first orthogonal cleavable linker and the second orthogonal cleavable linker are cleavable under different conditions. In embodiments, the first and the second cleavable linkers are cleavable under similar or identical conditions and the third and the fourth cleavable linkers are cleavable under similar or identical conditions.

In embodiments, the first nucleotide, the second nucleotide, the third nucleotide, and the fourth nucleotide independently include the formula:

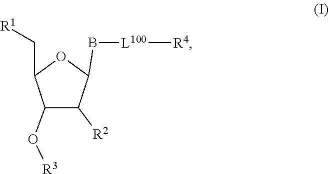

wherein B is a divalent nucleobase, R¹ is a polyphosphate moiety, monophosphate moiety, or nucleic acid moiety; R² is hydrogen or —OH; R³ is a reversible terminator; L¹⁰⁰ is a cleavable linker; and R⁴ is a fluorophore moiety.

In embodiments, the first nucleotide, the second nucleotide, the third nucleotide, and the fourth nucleotide each independently include the formula:

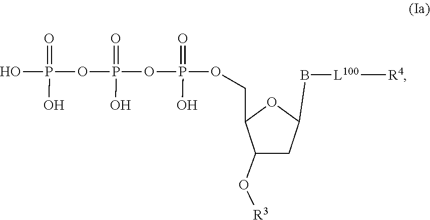

wherein B is a divalent nucleobase, R³ is a reversible terminator; L¹⁰⁰ is a cleavable linker; and R⁴ is a fluorophore moiety.

In embodiments, B is a divalent nucleobase. In embodiments, B is

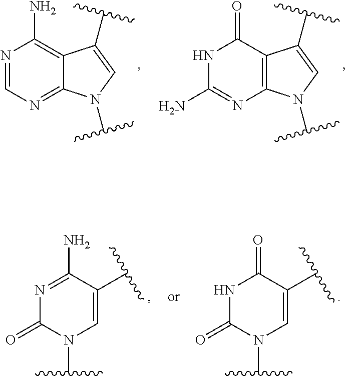 [SMILES: *c1cn(*)c2ncnc(N)c12 ; *c1cn(*)c2nc(N)[nH]c(=O)c12 ; *c1cn(*)c(=O)nc1N ; *c1cn(*)c(=O)[nH]c1=O]

In embodiments, B is

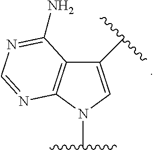 [SMILES: *c1cn(*)c2ncnc(N)c12]

In embodiments, B is

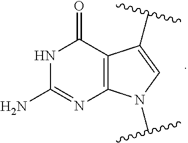 [SMILES: *c1cn(*)c2nc(N)[nH]c(=O)c12]

In embodiments, B is

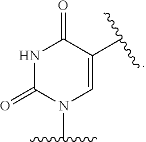 [SMILES: *c1cn(*)c(=O)[nH]c1=O]

In embodiments, B is

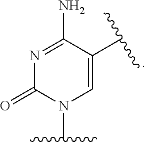 [SMILES: *c1cn(*)c(=O)nc1N]

In embodiments, B is a cytosine or a derivative thereof, guanine or a derivative thereof, adenine or a derivative thereof, thymine or a derivative thereof, uracil or a derivative thereof, hypoxanthine or a derivative thereof, xanthine or a derivative thereof, 7-methylguanine or a derivative thereof, 5,6-dihydrouracil or a derivative thereof, 5-methylcytosine or a derivative thereof, or 5-hydroxymethylcytosine or a derivative thereof. In embodiments, B is a substituted cytosine or a derivative thereof, substituted guanine or a derivative thereof, substituted adenine or a derivative thereof, substituted thymine or a derivative thereof, substituted uracil or a derivative thereof, substituted hypoxanthine or a derivative thereof, substituted xanthine or a derivative thereof, substituted 7-methylguanine or a derivative thereof, substituted 5,6-dihydrouracil or a derivative thereof, substituted 5-methylcytosine or a derivative thereof, or substituted 5-hydroxymethylcytosine or a derivative thereof. In embodiments, B is a substituted cytosine, substituted guanine, substituted adenine, substituted thymine, substituted uracil, substituted hypoxanthine, substituted xanthine, substituted 7-methylguanine, substituted 5,6-dihydrouracil, substituted 5-methylcytosine, or a substituted 5-hydroxymethylcytosine. In embodiments, B a substituted B is substituted with at least one substituent group, size-limited substituent group, or lower substituent group. In embodiments, when B is substituted, it is substituted with at least one substituent group. In embodiments, when B is substituted, it is substituted with at least one size-limited substituent group. In embodiments, when B is substituted, it is substituted with at least one lower substituent group.

In embodiments, B is a divalent cytosine or a derivative thereof, divalent guanine or a derivative thereof, divalent adenine or a derivative thereof, divalent thymine or a derivative thereof, divalent uracil or a derivative thereof, divalent hypoxanthine or a derivative thereof, divalent xanthine or a derivative thereof, divalent 7-methylguanine or a derivative thereof, divalent 5,6-dihydrouracil or a derivative thereof, divalent 5-methylcytosine or a derivative thereof, or divalent 5-hydroxymethylcytosine or a derivative thereof. In embodiments, B is a divalent cytosine or a derivative thereof. In embodiments, B is a divalent guanine or a derivative thereof. In embodiments, B is a divalent adenine or a derivative thereof. In embodiments, B is a divalent thymine or a derivative thereof. In embodiments, B is a divalent uracil or a derivative thereof. In embodiments, B is a divalent hypoxanthine or a derivative thereof. In embodiments, B is a divalent xanthine or a derivative thereof. In embodiments, B is a divalent 7-methylguanine or a derivative thereof. In embodiments, B is a divalent 5,6-dihydrouracil or a derivative thereof. In embodiments, B is a divalent 5-methylcytosine or a derivative thereof. In embodiments, B is a divalent 5-hydroxymethylcytosine or a derivative thereof. In embodiments, B is a divalent cytosine. In embodiments, B is a divalent guanine. In embodiments, B is a divalent adenine. In embodiments, B is a divalent thymine. In embodiments, B is a divalent uracil. In embodiments, B is a divalent hypoxanthine. In embodiments, B is a divalent xanthine. In embodiments, B is a divalent 7-methylguanine. In embodiments, B is a divalent 5,6-dihydrouracil. In embodiments, B is a divalent 5-methylcytosine. In embodiments, B is a divalent 5-hydroxymethylcytosine.

In embodiments, R¹ is a polyphosphate moiety. In embodiments, R¹ is a triphosphate moiety. In embodiments, R¹ is a monophosphate moiety. In embodiments, R¹ is a nucleic acid moiety. In embodiments, R¹ includes one or more phosphate moieties. In embodiments, R¹ includes one or more phosphorothioate moieties.

In embodiments, R² is hydrogen. In embodiments, R² is-OH.

In embodiments, R³ is a reversible terminator moiety (e.g., a reversible terminator moiety known in the art). In embodiments, R³ is a —O-reversible terminator moiety, wherein the —O— is attached to the 3′ position of the ribose sugar of a nucleotide and a reversible terminator moiety is as described herein. In embodiments, R³ is

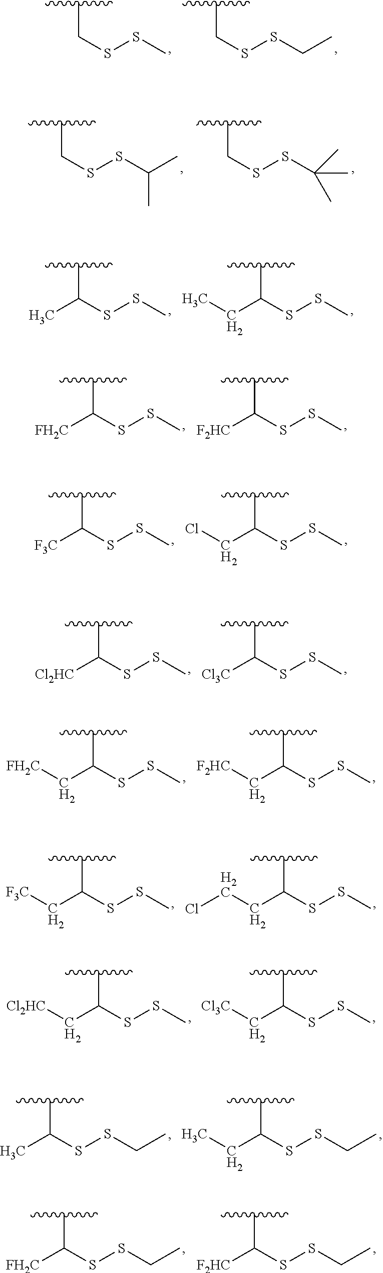

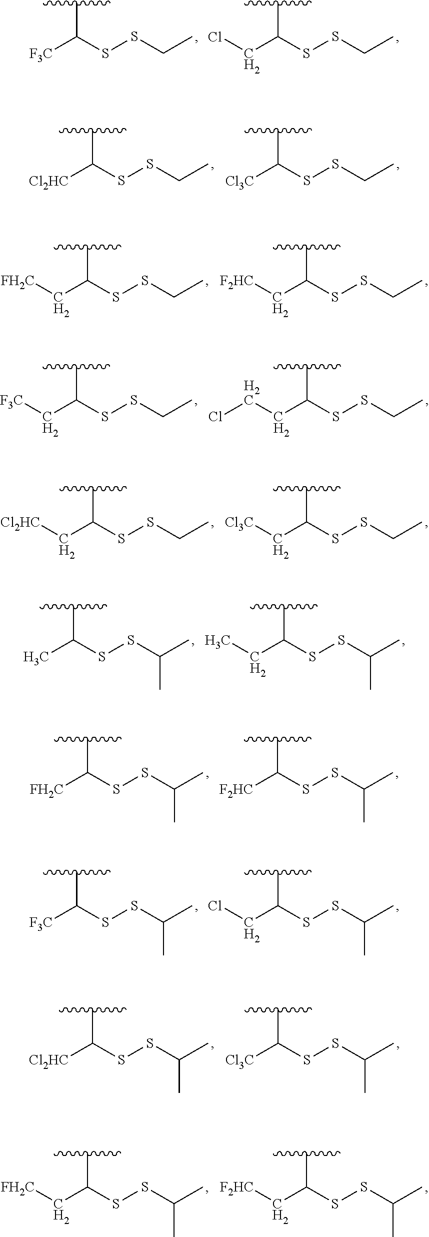

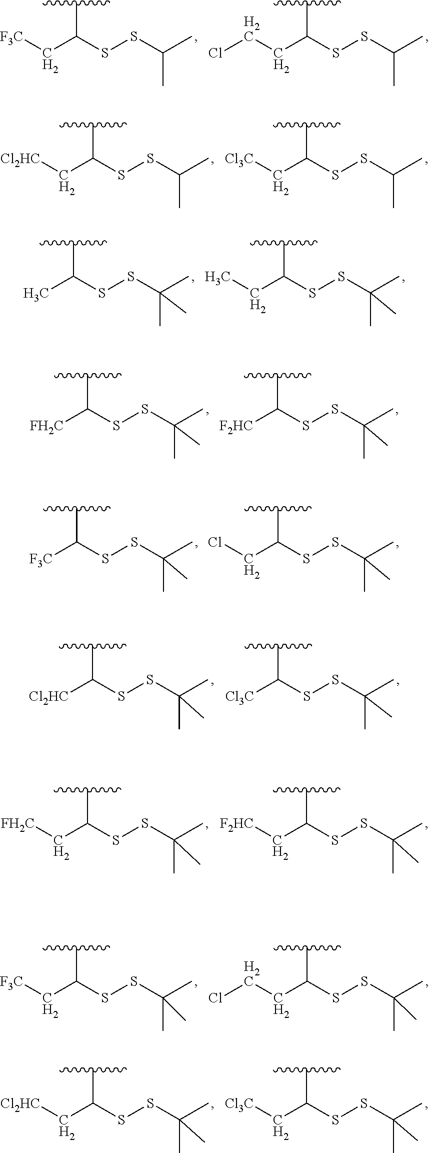

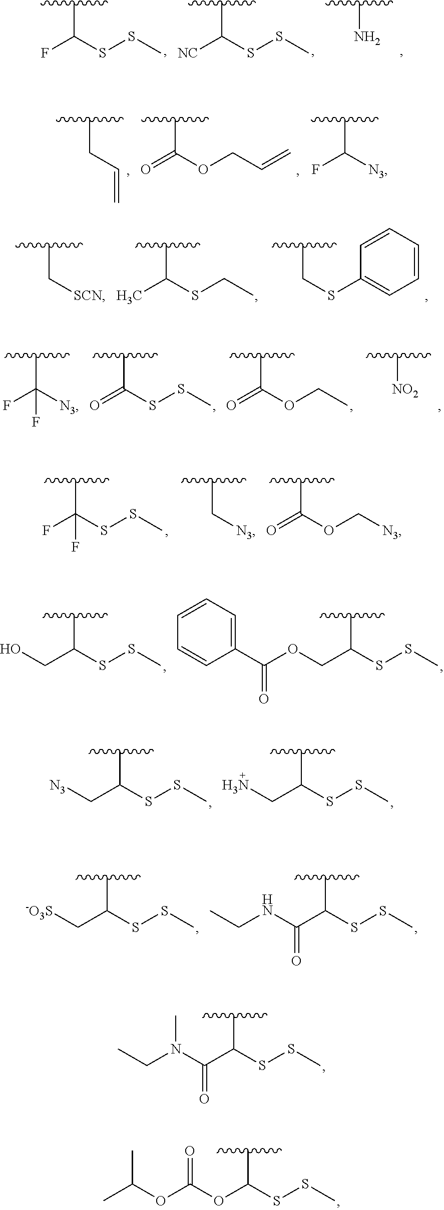

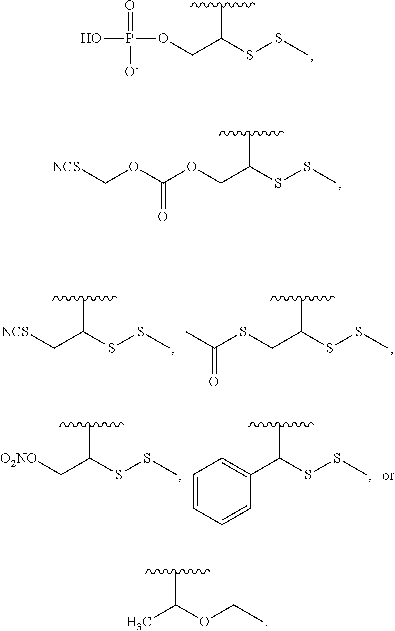

In embodiments, the polymerase-compatible cleavable moiety is:

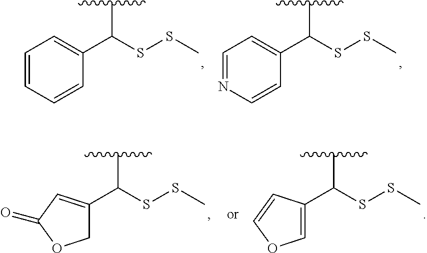

In embodiments, the reversible terminator moiety is

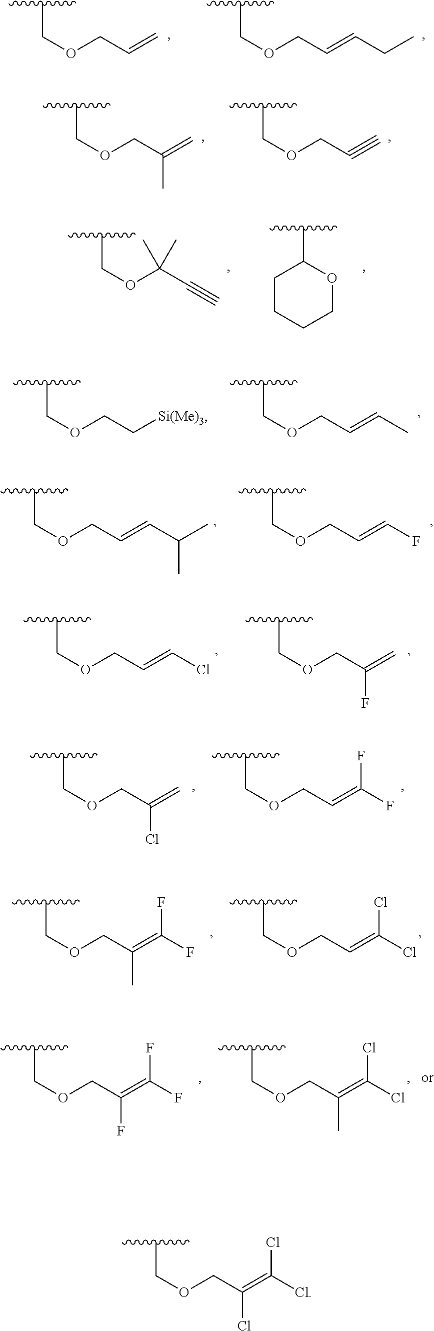

In embodiments, the reversible terminator moiety is

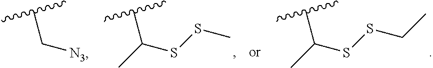

In embodiments, the reversible terminator moiety is

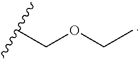 [SMILES: *COCC]

In embodiments, the reversible terminator moiety includes a halogen (e.g., —F). In embodiments, the reversible terminator moiety includes a —CN moiety. In embodiments, the reversible terminator moiety includes a —N₃ moiety. In embodiments, the reversible terminator moiety includes a —F moiety. In embodiments, the reversible terminator moiety includes a —Cl moiety. In embodiments, the reversible terminator moiety includes a —Br moiety. In embodiments, the reversible terminator moiety includes a —I moiety.

In embodiments, L¹⁰⁰ is a cleavable linker. In embodiments, L¹⁰⁰ is an orthogonal cleavable linker. In embodiments, the first cleavable linker attached to the first fluorophore moiety of two of the four nucleotides is an orthogonal cleavable linker relative to the second cleavable linker attached to the second fluorophore moiety of two of the four nucleotides.

In embodiments, L¹⁰⁰ is a bond, —NH—, —S—, —O—, —C(O)—, —C(O)O—, —OC(O)—, —NHC(O)—,
″\*MERGEFORMAT\*MERGEFORMAT —C(O)NH—, —NHC(O)NH—, —NHC(NH)NH—, —C(S)—, substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20, 8 to 20, 2 to 10, 2 to 8, 2 to 6, or 2 to 4 membered), substituted or unsubstituted cycloalkylene (e.g., C₃-C₈, C₃-C₆, or C₅-C₆), substituted or unsubstituted heterocycloalkylene (e.g., 3 to 8, 3 to 6, or 5 to 6 membered), substituted or unsubstituted arylene (e.g., C₆-C₁₀, C₁₀, or phenylene), or substituted or unsubstituted heteroarylene (e.g., 5 to 10, 5 to 9, or 5 to 6 membered). In embodiments, L¹⁰⁰ is not a bond.

In embodiments, L¹⁰⁰ is substituted (e.g., substituted alkylene, substituted heteroalkylene, substituted cycloalkylene, substituted heterocycloalkylene, substituted arylene, and/or substituted heteroarylene) with at least one substituent group, size-limited substituent group, or lower substituent group; wherein if the substituted L¹⁰⁰ is substituted with a plurality of groups selected from substituent groups, size-limited substituent groups, and lower substituent groups; each substituent group, size-limited substituent group, and/or lower substituent group may optionally be different. In embodiments, when L¹⁰⁰ is substituted, it is substituted with at least one substituent group. In embodiments, when L¹⁰⁰ is substituted, it is substituted with at least one size-limited substituent group. In embodiments, when L¹⁰⁰ is substituted, it is substituted with at least one lower substituent group.

In embodiments, L¹⁰⁰ is a bond, —NH—, —S—, —O—, —C(O)—, —C(O)O—, —OC(O)—, —NHC(O)—, —C(O)NH—, —NHC(O)NH—, —NHC(NH)NH—, —C(S)—, —N═N—, substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20, 8 to 20, 2 to 10, 2 to 8, 2 to 6, or 2 to 4 membered), substituted or unsubstituted cycloalkylene (e.g., C₃-C₈, C₃-C₆, or C₅-C₆), substituted or unsubstituted heterocycloalkylene (e.g., 3 to 8, 3 to 6, or 5 to 6 membered), substituted or unsubstituted arylene (e.g., C₆-C₁₀, C₁₀, or phenylene), or substituted or unsubstituted heteroarylene (e.g., 5 to 10, 5 to 9, or 5 to 6 membered). In embodiments, L¹⁰⁰ is a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L¹⁰⁰ is R¹⁰⁰-substituted or unsubstituted alkylene (e.g., C₁-C₂₀, C₁₀-C₂₀, C₁-C₈, C₁-C₆, or C₁-C₄), R¹⁰⁰-substituted or unsubstituted heteroalkylene (e.g., 2 to 20, 8 to 20, 2 to 10, 2 to 8, 2 to 6, or 2 to 4 membered), R¹⁰⁰-substituted or unsubstituted cycloalkylene (e.g., C₃-C₈, C₃-C₆, or C₅-C₆), R¹⁰⁰-substituted or unsubstituted heterocycloalkylene (e.g., 3 to 8, 3 to 6, or 5 to 6 membered), R¹⁰⁰-substituted or unsubstituted arylene (e.g., C₆-C₁₀, C₁₀, or phenylene), or R¹⁰⁰-substituted or unsubstituted heteroarylene (e.g., 5 to 10, 5 to 9, or 5 to 6 membered).

R¹⁰⁰ is oxo, halogen, —CCl₃, —CBr₃, —CF₃, —CI₃, —CHCl₂, —CHBr₂, —CHF₂,
″\*MERGEFORMAT\*MERGEFORMAT —CHI₂, —CH₂Cl, —CH₂Br, —CH₂F, —CH₂I, —CN, —OH, —NH₂, —COOH, —CONH₂, —NO₂, —SH, —SO₃H,
″\*MERGEFORMAT\*MERGEFORMAT —SO₄H, —SO₂NH₂, —NHNH₂, —ONH₂, —NHC(O)NHNH₂, —NHC(O)NH₂, —NHSO₂H, —NHC(O)H,
″\*MERGEFORMAT\*MERGEFORMAT —NHC(O)OH, —NHOH, —OCCl₃, —OCF₃, —OCBr₃, —OCI₃, —OCHCl₂, —OCHBr₂, —OCHI₂, —OCHF₂,
″\*MERGEFORMAT\*MERGEFORMAT —OCH₂Cl, —OCH₂Br, —OCH₂1, —OCH₂F, —N₃, —SFs, —NH₃+, —SO₃, —OPO₃H, —SCN, —ONO₂, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, L¹⁰⁰ is -L¹⁰¹-L¹⁰²-L¹⁰³-L¹⁰⁴-L¹⁰⁵-, wherein L¹⁰¹, L¹⁰², L¹⁰³, L¹⁰⁴, and L¹⁰⁵ are independently a bond, —NH—, —O—, —C(O)—, —C(O)NH—, —NHC(O)—, —NHC(O)NH—,
″\*MERGEFORMAT\*MERGEFORMAT —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L¹⁰¹ is substituted (e.g., substituted alkylene, substituted heteroalkylene, substituted cycloalkylene, substituted heterocycloalkylene, substituted arylene, and/or substituted heteroarylene) with at least one substituent group, size-limited substituent group, or lower substituent group; wherein if the substituted L¹⁰¹ is substituted with a plurality of groups selected from substituent groups, size-limited substituent groups, and lower substituent groups; each substituent group, size-limited substituent group, and/or lower substituent group may optionally be different. In embodiments, when L¹⁰¹ is substituted, it is substituted with at least one substituent group. In embodiments, when L¹⁰¹ is substituted, it is substituted with at least one size-limited substituent group. In embodiments, when L¹⁰¹ is substituted, it is substituted with at least one lower substituent group.

In embodiments, L¹⁰¹ is R¹⁰¹-substituted or unsubstituted alkylene, R¹⁰¹-substituted or unsubstituted heteroalkylene, R¹⁰¹-substituted or unsubstituted cycloalkylene, R¹⁰¹-substituted or unsubstituted heterocycloalkylene, R¹⁰¹-substituted or unsubstituted arylene, or R¹⁰¹-substituted or unsubstituted heteroarylene. In embodiments, L¹⁰¹ is R¹⁰¹-substituted or unsubstituted alkylene or R¹⁰¹-substituted or unsubstituted heteroalkylene. In embodiments, L¹⁰¹ is unsubstituted alkylene or unsubstituted heteroalkylene.

R¹⁰¹ is independently oxo, halogen, —CCl₃, —CBr₃, —CF₃, —CI₃, —CHCl₂, —CHBr₂, —CHF₂,
″\*MERGEFORMAT\*MERGEFORMAT —CHI₂, —CH₂Cl, —CH₂Br, —CH₂F, —CH₂I, —CN, —OH, —NH₂, —COOH, —CONH₂, —NO₂, —SH, —SO₃H,
″\*MERGEFORMAT\*MERGEFORMAT —SO₄H, —SO₂NH₂, —NHNH₂, —ONH₂, —NHC(O)NHNH₂, —NHC(O)NH₂, —NHSO₂H, —NHC(O)H,
″\*MERGEFORMAT\*MERGEFORMAT —NHC(O)OH, —NHOH, —OCCl₃, —OCF₃, —OCBr₃, —OCI₃, —OCHCl₂, —OCHBr₂, —OCHI₂, —OCHF₂,
″\*MERGEFORMAT\*MERGEFORMAT —OCH₂Cl, —OCH₂Br, —OCH₂1, —OCH₂F, —N₃, —SF₅, —NH₃+, —SO₃, —OPO₃H, —SCN, —ONO₂, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, L¹⁰² is substituted (e.g., substituted alkylene, substituted heteroalkylene, substituted cycloalkylene, substituted heterocycloalkylene, substituted arylene, and/or substituted heteroarylene) with at least one substituent group, size-limited substituent group, or lower substituent group; wherein if the substituted L¹⁰² is substituted with a plurality of groups selected from substituent groups, size-limited substituent groups, and lower substituent groups; each substituent group, size-limited substituent group, and/or lower substituent group may optionally be different. In embodiments, when L¹⁰² is substituted, it is substituted with at least one substituent group. In embodiments, when L¹⁰² is substituted, it is substituted with at least one size-limited substituent group. In embodiments, when L¹⁰² is substituted, it is substituted with at least one lower substituent group.

In embodiments, L¹⁰² is R¹⁰²-substituted or unsubstituted alkylene, R¹⁰²-substituted or unsubstituted heteroalkylene, R¹⁰²-substituted or unsubstituted cycloalkylene, R¹⁰²-substituted or unsubstituted heterocycloalkylene, R¹⁰²-substituted or unsubstituted arylene, or R¹⁰²-substituted or unsubstituted heteroarylene. In embodiments, L¹⁰² is R¹⁰²-substituted or unsubstituted alkylene or R¹⁰²-substituted or unsubstituted heteroalkylene. In embodiments, L¹⁰² is unsubstituted alkylene or unsubstituted heteroalkylene.

R¹⁰² is independently oxo, halogen, —CCl₃, —CBr₃, —CF₃, —CI₃, —CHCl₂, —CHBr₂, —CHF₂,
″\*MERGEFORMAT\*MERGEFORMAT —CHI₂, —CH₂Cl, —CH₂Br, —CH₂F, —CH₂I, —CN, —OH, —NH₂, —COOH, —CONH₂, —NO₂, —SH, —SO₃H,
″\*MERGEFORMAT\*MERGEFORMAT —SO₄H, —SO₂NH₂, —NHNH₂, —ONH₂, —NHC(O)NHNH₂, —NHC(O)NH₂, —NHSO₂H, —NHC(O)H,
″\*MERGEFORMAT\*MERGEFORMAT —NHC(O)OH, —NHOH, —OCCl₃, —OCF₃, —OCBr₃, —OCI₃, —OCHCl₂, —OCHBr₂, —OCHI₂, —OCHF₂,
″\*MERGEFORMAT\*MERGEFORMAT —OCH₂Cl, —OCH₂Br, —OCH₂I, —OCH₂F, —N₃, —SF₅, —NH₃+, —SO₃, —OPO₃H, —SCN, —ONO₂, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, L¹⁰³ is substituted (e.g., substituted alkylene, substituted heteroalkylene, substituted cycloalkylene, substituted heterocycloalkylene, substituted arylene, and/or substituted heteroarylene) with at least one substituent group, size-limited substituent group, or lower substituent group; wherein if the substituted L¹⁰³ is substituted with a plurality of groups selected from substituent groups, size-limited substituent groups, and lower substituent groups; each substituent group, size-limited substituent group, and/or lower substituent group may optionally be different. In embodiments, when L¹⁰³ is substituted, it is substituted with at least one substituent group. In embodiments, when L¹⁰³ is substituted, it is substituted with at least one size-limited substituent group. In embodiments, when L¹⁰³ is substituted, it is substituted with at least one lower substituent group.

In embodiments, L¹⁰³ is R¹⁰³-substituted or unsubstituted alkylene, R¹⁰³-substituted or unsubstituted heteroalkylene, R¹⁰³-substituted or unsubstituted cycloalkylene, R¹⁰³-substituted or unsubstituted heterocycloalkylene, R¹⁰³-substituted or unsubstituted arylene, or R¹⁰³-substituted or unsubstituted heteroarylene. In embodiments, L¹⁰³ is R¹⁰³-substituted or unsubstituted alkylene or R¹⁰³-substituted or unsubstituted heteroalkylene. In embodiments, L¹⁰³ is unsubstituted alkylene or unsubstituted heteroalkylene.

R¹⁰³ is independently oxo, halogen, —CCl₃, —CBr₃, —CF₃, —CI₃, —CHCl₂, —CHBr₂, —CHF₂,
″\*MERGEFORMAT\*MERGEFORMAT —CHI₂, —CH₂Cl, —CH₂Br, —CH₂F, —CH₂I, —CN, —OH, —NH₂, —COOH, —CONH₂, —NO₂, —SH, —SO₃H,
″\*MERGEFORMAT\*MERGEFORMAT —SO₄H, —SO₂NH₂, —NHNH₂, —ONH₂, —NHC(O)NHNH₂, —NHC(O)NH₂, —NHSO₂H, —NHC(O)H,
″\*MERGEFORMAT\*MERGEFORMAT —NHC(O)OH, —NHOH, —OCCl₃, —OCF₃, —OCBr₃, —OCI₃, —OCHCl₂, —OCHBr₂, —OCHI₂, —OCHF₂,
″\*MERGEFORMAT\*MERGEFORMAT —OCH₂Cl, —OCH₂Br, —OCH₂I, —OCH₂F, —N₃, —SFs, —NH₃+, —SO₃, —OPO₃H, —SCN, —ONO₂, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, L¹⁰⁴ is substituted (e.g., substituted alkylene, substituted heteroalkylene, substituted cycloalkylene, substituted heterocycloalkylene, substituted arylene, and/or substituted heteroarylene) with at least one substituent group, size-limited substituent group, or lower substituent group; wherein if the substituted L¹⁰⁴ is substituted with a plurality of groups selected from substituent groups, size-limited substituent groups, and lower substituent groups; each substituent group, size-limited substituent group, and/or lower substituent group may optionally be different. In embodiments, when L¹⁰⁴ is substituted, it is substituted with at least one substituent group. In embodiments, when L¹⁰⁴ is substituted, it is substituted with at least one size-limited substituent group. In embodiments, when L¹⁰⁴ is substituted, it is substituted with at least one lower substituent group.

In embodiments, L¹⁰⁴ is R¹⁰⁴-substituted or unsubstituted alkylene, R¹⁰⁴-substituted or unsubstituted heteroalkylene, R¹⁰⁴-substituted or unsubstituted cycloalkylene, R¹⁰⁴-substituted or unsubstituted heterocycloalkylene, R¹⁰⁴-substituted or unsubstituted arylene, or R¹⁰⁴-substituted or unsubstituted heteroarylene. In embodiments, L¹⁰⁴ is R¹⁰⁴-substituted or unsubstituted alkylene or R¹⁰⁴-substituted or unsubstituted heteroalkylene. In embodiments, L¹⁰ 4 is unsubstituted alkylene or unsubstituted heteroalkylene.

R¹⁰⁴ is independently oxo, halogen, —CCl₃, —CBr₃, —CF₃, —CI₃, —CHCl₂, —CHBr₂, —CHF₂,
″\*MERGEFORMAT\*MERGEFORMAT —CHI₂, —CH₂Cl, —CH₂Br, —CH₂F, —CH₂I, —CN, —OH, —NH₂, —COOH, —CONH₂, —NO₂, —SH, —SO₃H,
″\*MERGEFORMAT\*MERGEFORMAT —SO₄H, —SO₂NH₂, —NHNH₂, —ONH₂, —NHC(O)NHNH₂, —NHC(O)NH₂, —NHSO₂H, —NHC(O)H,
″\*MERGEFORMAT\*MERGEFORMAT —NHC(O)OH, —NHOH, —OCCl₃, —OCF₃, —OCBr₃, —OCI₃, —OCHCl₂, —OCHBr₂, —OCHI₂, —OCHF₂,
″\*MERGEFORMAT\*MERGEFORMAT —OCH₂Cl, —OCH₂Br, —OCH₂I, —OCH₂F, —N₃, —SF₅, —NH₃+, —SO₃, —OPO₃H, —SCN, —ONO₂, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, L¹⁰⁵ is substituted (e.g., substituted alkylene, substituted heteroalkylene, substituted cycloalkylene, substituted heterocycloalkylene, substituted arylene, and/or substituted heteroarylene) with at least one substituent group, size-limited substituent group, or lower substituent group; wherein if the substituted L¹⁰⁵ is substituted with a plurality of groups selected from substituent groups, size-limited substituent groups, and lower substituent groups; each substituent group, size-limited substituent group, and/or lower substituent group may optionally be different. In embodiments, when L¹⁰⁵ is substituted, it is substituted with at least one substituent group. In embodiments, when L¹⁰⁵ is substituted, it is substituted with at least one size-limited substituent group. In embodiments, when L¹⁰⁵ is substituted, it is substituted with at least one lower substituent group.

In embodiments, L¹⁰⁵ is R¹⁰⁵-substituted or unsubstituted alkylene, R¹⁰⁵-substituted or unsubstituted heteroalkylene, R¹⁰⁵-substituted or unsubstituted cycloalkylene, R¹⁰⁵-substituted or unsubstituted heterocycloalkylene, R¹⁰⁵-substituted or unsubstituted arylene, or R¹⁰⁵-substituted or unsubstituted heteroarylene. In embodiments, L¹⁰⁵ is R¹⁰⁵-substituted or unsubstituted alkylene or R¹⁰⁵-substituted or unsubstituted heteroalkylene. In embodiments, L¹⁰⁵ is unsubstituted alkylene or unsubstituted heteroalkylene.

R¹⁰⁵ is independently oxo, halogen, —CCl₃, —CBr₃, —CF₃, —CI₃, —CHCl₂, —CHBr₂, —CHF₂,
″\*MERGEFORMAT\*MERGEFORMAT —CHI₂, —CH₂Cl, —CH₂Br, —CH₂F, —CH₂I, —CN, —OH, —NH₂, —COOH, —CONH₂, —NO₂, —SH, —SO₃H,
″\*MERGEFORMAT\*MERGEFORMAT —SO₄H, —SO₂NH₂, —NHNH₂, —ONH₂, —NHC(O)NHNH₂, —NHC(O)NH₂, —NHSO₂H, —NHC(O)H,
″\*MERGEFORMAT\*MERGEFORMAT —NHC(O)OH, —NHOH, —OCCl₃, —OCF₃, —OCBr₃, —OCI₃, —OCHCl₂, —OCHBr₂, —OCHI₂, —OCHF₂,
″\*MERGEFORMAT\*MERGEFORMAT —OCH₂Cl, —OCH₂Br, —OCH₂1, —OCH₂F, —N₃, —SF₅, —NH₃+, —SO₃, —OPO₃H, —SCN, —ONO₂, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, L¹⁰⁰ is a divalent linker including

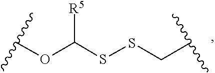

wherein R⁵ is described herein. In embodiments, L¹⁰⁰ is a divalent linker including

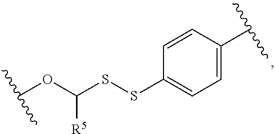

wherein R⁵ is described herein.

In embodiments, L¹⁰⁰ is a divalent linker including

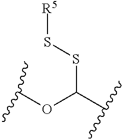

wherein R⁵ is described herein. In embodiments, L¹⁰⁰ is a divalent linker including,

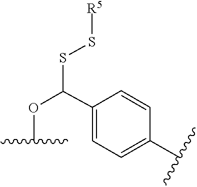

wherein R⁵ is described herein. In embodiments, L¹⁰⁰ is a divalent linker including

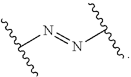 [SMILES: */N=N/*]

In embodiments, L¹⁰⁰ is a divalent linker including

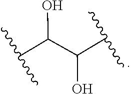 [SMILES: *C(O)C(*)O]

In embodiments, L¹⁰⁰ is a divalent linker including

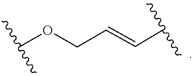 [SMILES: */C=C/CO*]

In embodiments, L¹⁰⁰ is a divalent linker including

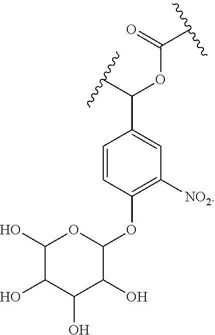 [SMILES: *C(=O)OC(*)c1ccc(OC2OC(O)C(O)C(O)C2O)c([N+](=O)[O-])c1]

In embodiments, L¹⁰⁰ is an enzymatically cleavable linker, wherein L¹⁰⁰ includes a Cathepsin B substrate moiety (e.g., a moiety including

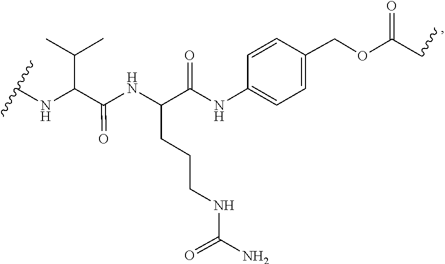

valine-citrulline dipeptide, alanine-citrulline dipeptide, z-Arginine-Arginine-para-nitroanalide, or z-Arginine-Arginine-amino-4-methylcoumarin as described in Zheng, Su. Acta Pharmaceutica Sinica B. 2021 December; 11(12): 3889-3907 and Yoon, M. et al. Biochemistry. 2023 Aug. 1; 62(15): 2289-2300), β-glucuronidase substrate moiety (e.g., a moiety including

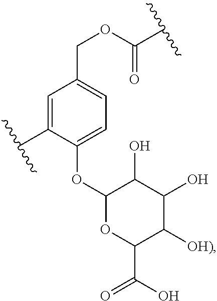

β-galactosidase substrate moiety (e.g., a moiety including

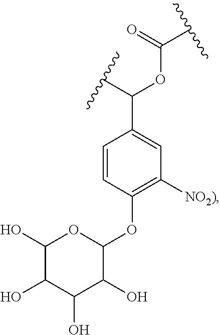

a sulfatase substrate moiety (e.g., a moiety including

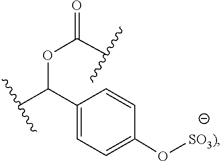

or derivatives thereof. In embodiments, L¹⁰⁰ is a divalent enzymatically cleavable linker including a β-galactosidase substrate moiety. In embodiments, L¹⁰⁰ is a divalent linker that is cleavable in the presence of a reducing agent, wherein L¹⁰⁰ includes a disulfide moiety or an azo moiety. In embodiments, L¹⁰⁰ is a divalent linker that is cleavable in the presence of an oxidative agent, wherein L¹⁰⁰ includes vicinal (i.e., 1,2-diol) diol moiety or selenium containing moiety. In embodiments, L¹⁰⁰ is a divalent linker that is a photocleavable linker, wherein L¹⁰⁰ includes 2-nitrobenzyl moiety (i.e., ortho-nitrobenzyl), phenacyl ester moiety, 8-quinolinyl benzenesulfonate moiety, or dicoumarin moiety, or bis-arylhydrazone moiety. In embodiments, L¹⁰⁰ is a divalent linker that is cleavable in the presence of a base, wherein L¹⁰⁰ includes cyanoethyl moiety or thioester moiety. In embodiments, L¹⁰⁰ is a divalent linker that is cleavable in the presence of an acid, wherein L¹⁰⁰ includes an acetal moiety, cyclic acetal moiety, dialkylketal moiety, silyl ether moiety, or hydrazone moiety (see, e.g., Leriche, G. et al. Bioorg Med Chem. 2012 Jan. 15; 20(2):571-82). In embodiments, L¹⁰⁰ is a divalent linker including a polynucleotide sequence. In embodiments, L¹⁰⁰ is a divalent linker including a polypeptide sequence (e.g., a linker including a 15-residue (Gly₄Ser)₃ peptide).

In embodiments, L¹⁰⁰ is a divalent linker from the first and second nucleotide as described herein that includes reductant-cleavable moiety, and L¹⁰⁰ is a divalent linker from the third and fourth nucleotide as described herein that includes an oxidant-cleavable moiety. In embodiments, L¹⁰⁰ is a divalent linker from the first and second nucleotide as described herein that includes reductant-cleavable moiety, and L¹⁰⁰ is a divalent linker from the third and fourth nucleotide as described herein that includes an enzyme-cleavable moiety. In embodiments, L¹⁰⁰ is a divalent linker from the first and second nucleotide as described herein that includes a disulfide moiety, and L¹⁰⁰ is a divalent linker from the third and fourth nucleotide as described herein that includes a β-galactosidase substrate moiety. In embodiments, L¹⁰⁰ is a divalent linker from the first and second nucleotide as described herein that includes a disulfide moiety, and L¹⁰⁰ is a divalent linker from the third and fourth nucleotide as described herein that includes a vicinal (i.e., 1,2-diol) diol moiety.

In embodiments, R⁵ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁵ is a substituted (e.g., substituted alkyl, substituted heteroalkyl, substituted cycloalkyl, substituted heterocycloalkyl, substituted aryl, and/or substituted heteroaryl) is substituted with at least one substituent group, size-limited substituent group, or lower substituent group; wherein if the substituted R⁵ is substituted with a plurality of groups selected from substituent groups, size-limited substituent groups, and lower substituent groups; each substituent group, size-limited substituent group, and/or lower substituent group may optionally be different. In embodiments, when R⁵ is substituted, it is substituted with at least one substituent group. In embodiments, when R⁵ is substituted, it is substituted with at least one size-limited substituent group. In embodiments, when R⁵ is substituted, it is substituted with at least one lower substituent group.

In embodiments, R⁵ is R⁵⁰⁰-substituted or unsubstituted alkyl (e.g., C₁-C₂₀, C₁₀-C₂₀,
″\*MERGEFORMAT\*MERGEFORMAT C₁-C₈, C₁-C₆, or C₁-C₄), R⁵⁰⁰-substituted or unsubstituted heteroalkyl (e.g., 2 to 20, 8 to 20, 2 to 10, 2 to 8, 2 to 6, or 2 to 4 membered), R⁵⁰⁰-substituted or unsubstituted cycloalkyl (e.g., C₃-C₈, C₃-C₆, or C₅-C₆), R⁵⁰⁰-substituted or unsubstituted heterocycloalkyl (e.g., 3 to 8, 3 to 6, or 5 to 6 membered), R⁵⁰⁰-substituted or unsubstituted aryl (e.g., C₆-C₁₀, C₁₀, or phenyl), or
″\*MERGEFORMAT\*MERGEFORMAT R⁵⁰⁰-substituted or unsubstituted heteroaryl (e.g., 5 to 10, 5 to 9, or 5 to 6 membered). In embodiments, R⁵ is substituted or unsubstituted alkyl (e.g., C₁-C₂₀, C₁₀-C₂₀, C₁-C₈, C₁-C₆, or
″\*MERGEFORMAT\*MERGEFORMAT C₁-C₄), substituted or unsubstituted heteroalkyl (e.g., 2 to 20, 8 to 20, 2 to 10, 2 to 8, 2 to 6, or 2 to 4 membered), substituted or unsubstituted cycloalkyl (e.g., C₃-C₈, C₃-C₆, or C₅-C₆), substituted or unsubstituted heterocycloalkyl (e.g., 3 to 8, 3 to 6, or 5 to 6 membered), substituted or unsubstituted aryl (e.g., C₆-C₁₀, C₁₀, or phenyl), or substituted or unsubstituted heteroaryl (e.g., 5 to 10, 5 to 9, or 5 to 6 membered).

In embodiments, R⁵ is R⁵⁰⁰-substituted or unsubstituted alkyl (e.g., C₁-C₂₀, C₁₀-C₂₀,
″\*MERGEFORMAT\*MERGEFORMAT C₁-C₈, C₁-C₆, or C₁-C₄). In embodiments, R⁵ is R⁵⁰⁰-substituted or unsubstituted C₁-C₂₀ alkyl. In embodiments, R⁵ is R⁵⁰⁰-substituted or unsubstituted C₁₀-C₂₀ alkyl. In embodiments, R⁵ is R⁵⁰⁰-substituted or unsubstituted C₁-C₈ alkyl. In embodiments, R⁵ is R⁵⁰⁰-substituted or unsubstituted C₁-C₆ alkyl. In embodiments, R⁵ is R⁵⁰⁰-substituted or unsubstituted C₁-C₄ alkyl.

In embodiments, R⁵ is unsubstituted C₁-C₄ alkyl. In embodiments, R⁵ is unsubstituted methyl. In embodiments, R⁵ is unsubstituted ethyl. In embodiments, R⁵ is unsubstituted propyl. In embodiments, R⁵ is unsubstituted butyl.

In embodiments, R⁵ is R⁵⁰⁰-substituted or unsubstituted heteroalkyl (e.g., 2 to 20, 8 to 20, 2 to 10, 2 to 8, 2 to 6, or 2 to 4 membered). In embodiments, R⁵ is R⁵⁰⁰-substituted or unsubstituted 2 to 20 membered heteroalkyl. In embodiments, R⁵ is R⁵⁰⁰-substituted or unsubstituted 8 to 20 membered heteroalkyl. In embodiments, R⁵ is R⁵⁰⁰-substituted or unsubstituted 2 to 10 membered heteroalkyl. In embodiments, R⁵ is R⁵⁰⁰-substituted or unsubstituted 2 to 8 membered heteroalkyl. In embodiments, R⁵ is R⁵⁰⁰-substituted or unsubstituted 2 to 6 membered heteroalkyl. In embodiments, R⁵ is R⁵⁰⁰-substituted or unsubstituted 2 to 4 membered heteroalkyl.

In embodiments, R⁵ is R⁵⁰⁰-substituted or unsubstituted cycloalkyl (e.g., C₃-C₈, C₃-C₆, or C₅-C₆). In embodiments, R⁵ is R⁵⁰⁰-substituted or unsubstituted C₃-C₈ cycloalkyl. In embodiments, R⁵ is R⁵⁰⁰-substituted or unsubstituted C₃-C₆ cycloalkyl. In embodiments, R⁵ is R⁵⁰⁰-substituted or unsubstituted C₅-C₆ cycloalkyl.

In embodiments, R⁵ is R⁵⁰⁰-substituted or unsubstituted heterocycloalkyl (e.g., 3 to 8, 3 to 6, or 5 to 6 membered). In embodiments, R⁵ is R⁵⁰⁰-substituted or unsubstituted 3 to 8 heterocycloalkyl. In embodiments, R⁵ is R⁵⁰⁰-substituted or unsubstituted 3 to 6 heterocycloalkyl. In embodiments, R⁵ is R⁵⁰⁰-substituted or unsubstituted 5 to 6 heterocycloalkyl.

In embodiments, R⁵ is R⁵⁰⁰-substituted or unsubstituted aryl (e.g., C₆-C₁₀, C₁₀, or phenyl). In embodiments, R⁵ is R⁵⁰⁰-substituted or unsubstituted C₆-C₁₀ aryl. In embodiments, R⁵ is R⁵⁰⁰-substituted or unsubstituted C₁₀ aryl. In embodiments, R⁵ is R⁵⁰⁰-substituted or unsubstituted phenyl.

In embodiments, R⁵ is R⁵⁰⁰-substituted or unsubstituted heteroaryl (e.g., 5 to 10, 5 to 9, or 5 to 6 membered). In embodiments, R⁵ is R⁵⁰⁰-substituted or unsubstituted 5 to 10 membered heteroaryl. In embodiments, R⁵ is R⁵⁰⁰-substituted or unsubstituted 5 to 9 membered heteroaryl. In embodiments, R⁵ is R⁵⁰⁰-substituted or unsubstituted 5 to 6 membered heteroaryl.

R⁵⁰⁰ is oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H,
″\*MERGEFORMAT\*MERGEFORMAT —SO₂NH₂, —NHNH₂, —ONH₂, —NHC(O)NHNH₂, —NHC(O)NH₂, —NHSO₂H, —NHC(O)H,
″\*MERGEFORMAT\*MERGEFORMAT —NHC(O)OH, —NHOH, —OCCl₃, —OCF₃, —OCBr₃, —OCI₃, —OCHCl₂, —OCHBr₂, —OCHI₂, —OCHF₂,
″\*MERGEFORMAT\*MERGEFORMAT —OCH₂Cl, —OCH₂Br, —OCH₂I, —OCH₂F, —N₃, —SF₅, unsubstituted alkyl (e.g., C₁-C₂₀, C₁₀-C₂₀,
″\*MERGEFORMAT\*MERGEFORMAT C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

Fluorescent compounds and fluorophore moieties absorb light and then emit light instantaneously at a different wavelength, most of the times at a longer one. Fluorescent compounds convert all or part of the light (depending on the absorbance coefficient and quantum yield of the molecule) absorbed in a certain energy interval to radiate it at longer wavelengths. This approach is used to fabricate or modify light sources that emit in the visible spectral range (light wavelengths between 400 and 800 nm). These latter sources are used in lighting devices that produce visible light. Examples of such lighting devices are fluorescent tubes, fluorescent compact lamps, or ultraviolet-based white light emitting diodes, where the ultraviolet radiation, invisible to the human eye, is converted by fluorescent materials into visible light (longer than UV) with a spectral distribution between 400 and 800 nm.

In embodiments, R⁴ is a fluorophore moiety. In embodiments, R⁴ is a fluorescent moiety (e.g., acridine dye moiety, cyanine dye moiety, fluorine dye moiety, oxazine dye moiety, phenanthridine dye moiety, or rhodamine dye moiety). In embodiments, the R⁴ is a fluorescent moiety or fluorescent dye moiety. In embodiments, R⁴ is a triarylmethane moiety, sulforhodamine 101 moiety, sulforhodamine B moiety, Janelia Fluor® dye moiety, naphthalimide moiety, fluorescein isothiocyanate moiety, tetramethylrhodamine-5-(and 6)-isothiocyanate moiety, cyanine moiety, Cy2® moiety, Cy3® moiety, Cy5® moiety, Cy7® moiety, 4′,6-diamidino-2-phenylindole moiety, Hoechst 33258 moiety, Hoechst 33342 moiety, Hoechst 34580 moiety, propidium-iodide moiety, or acridine orange moiety. In embodiments, R⁴ is an Indo-1 Ca saturated moiety, Indo-1 Ca²⁺ moiety, Cascade Blue® BSA moiety, Cascade Blue® moiety, LysoTracker® Blue moiety, Alexa Fluor® 405 moiety, LysoSensor® Blue moiety, DyLight™ 405 moiety, DyLight™ 350 moiety, BFP (Blue Fluorescent Protein) moiety, Alexa Fluor® 350 moiety, coumarin moiety, 7-Amino-4-methylcoumarin moiety, Amino Coumarin moiety, AMCA conjugate moiety, Coumarin moiety, 7-Hydroxy-4-methylcoumarin moiety, 6,8-Difluoro-7-hydroxy-4-methylcoumarin moiety, Hoechst 33342 moiety, Pacific Blue™ moiety, Hoechst 33258 moiety, Pacific Blue™ antibody conjugate moiety, PO-PRO™-1 moiety, PO-PRO™-1-DNA moiety, POPO™-1 moiety, POPO™-1-DNA moiety, DAPI-DNA moiety, DAPI moiety, Marina Blue® moiety, SYTOX Blue™-DNA moiety, CFP (Cyan Fluorescent Protein) moiety, eCFP (Enhanced Cyan Fluorescent Protein) moiety, 1-Anilinonaphthalene-8-sulfonic acid (1,8-ANS) moiety, Indo-1, Ca free moiety, 1,8-ANS (1-Anilinonaphthalene-8-sulfonic acid) moiety, BO-PRO™-1-DNA moiety, BOPRO-1 moiety, BOBO™-1-DNA moiety, SYTO™ 45-DNA moiety, evoglow-Pp1 moiety, evoglow-Bs1 moiety, evoglow-Bs2 moiety, Auramine O moiety, DiO moiety, LysoSensor™ Green moiety, Cy2® moiety, Fura-2 high Ca moiety, SYTO™ 13-DNA moiety, YO-PRO™-1-DNA moiety, YOYO™-1-DNA moiety, eGFP (Enhanced Green Fluorescent Protein) moiety, LysoTracker™ Green moiety, GFP (S65T) moiety, BODIPY® FL, Sapphire moiety, BODIPY® FL conjugate moiety, MitoTracker™ Green moiety, MitoTracker™ Green FM™, Fluorescein moiety, Calcein moiety, Fura-2, no Ca moiety, Fluo-4 moiety, DTAF moiety, CFDA moiety, FITC moiety, Alexa Fluor® 488 hydrazide-water moiety, DyLight™ 488 moiety, 5-FAM moiety, Alexa Fluor® 488 moiety, Rhodamine 110 moiety, Acridine Orange moiety, BCECF moiety, PicoGreen® dsDNA quantitation reagent moiety, SYBR® Green I moiety, Rhodamine Green pH 7.0 moiety, CyQUANT™ GR-DNA moiety, NeuroTrace™ 500/525, green fluorescent Niss1 stain-RNA moiety, DansylCadaverine moiety, Fluoro-Emerald moiety, Niss1 moiety, Fluorescein dextran moiety, Rhodamine Green moiety, 5-(and-6)-Carboxy-2′, 7′-dichlorofluorescein moiety, DansylCadaverine, eYFP (Enhanced Yellow Fluorescent Protein) moiety, Oregon Green™ 488 moiety, Fluo-3 moiety, BCECF moiety, SBFI—Na⁺ moiety, Fluo-3 Ca²⁺ moiety, Rhodamine 123 moiety, FlAsH moiety, Calcium Green-1 Ca²⁺ moiety, Magnesium Green moiety, DM-NERF pH 4.0 moiety, Calcium Green moiety, Citrine moiety, LysoSensor® Yellow moiety, TO-PRO®-1-DNA moiety, Magnesium Green Mg²⁺ moiety, Sodium Green Na⁺ moiety, TOTO™-1-DNA moiety, Oregon Green™ 514 moiety, Oregon Green™ 514 antibody conjugate moiety, NBD-X moiety, DM-NERF pH 7.0 moiety, NBD-X, CI-NERF pH 6.0 moiety, Alexa Fluor® 430 moiety, CI-NERF pH 2.5 moiety, Lucifer Yellow, 6-TET, SE pH 9.0 moiety, Eosin antibody conjugate moiety, Eosin moiety, 6-Carboxyrhodamine 6G pH 7.0 moiety, 6-Carboxyrhodamine 6G, hydrochloride moiety, BODIPY® R6G SE moiety, BODIPY® R6G moiety, Cascade Yellow moiety, mBanana moiety, Alexa Fluor® 532 moiety, Erythrosin-5-isothiocyanate pH 9.0 moiety, 6-HEX, SE pH 9.0 moiety, mOrange moiety, mHoneydew moiety, Cy3® moiety, Rhodamine B moiety, DiI moiety, Alexa Fluor® 555 moiety, DyLight™ 549 moiety, BODIPY® TMR-X, SE moiety, BODIPY® TMR-X moiety, PO-PRO™-3-DNA moiety, PO-PRO™-3 moiety, Rhodamine moiety, POPO™-3 moiety, Alexa Fluor® 546 moiety, Calcium Orange Ca2+ moiety, TRITC moiety, Calcium Orange moiety, Rhodaminephalloidin pH 7.0 moiety, MitoTracker™ Orange moiety, MitoTracker™ Orange moiety, Phycoerythrin moiety, Magnesium Orange moiety, R-Phycoerythrin pH 7.5 moiety, 5-TAMRA™ moiety, Rhod-2 moiety, FM™ 1-43 moiety, Rhod-2 Ca²⁺ moiety, FM™ 1-43 lipid moiety, LOLO™-1-DNA moiety, dTomato moiety, DsRed moiety, Dapoxyl (2-aminoethyl) sulfonamide moiety, Tetramethylrhodamine dextran pH 7.0 moiety, Fluor-Ruby moiety, Resorufin moiety, Resorufin pH 9.0 moiety, mTangerine moiety, LysoTracker™ Red moiety, Lissamine rhodamine moiety, Cy3.5® moiety, Rhodamine Red-X antibody conjugate pH 8.0 moiety, Sulforhodamine 101 moiety, JC-1 pH 8.2 moiety, JC-1 moiety, mStrawberry moiety, MitoTracker™ Red moiety, MitoTracker™ Red, X-Rhod-1 Ca2+ moiety, Alexa Fluor® 568 moiety, 5-ROX™ pH 7.0 moiety, 5-ROX™ (5-Carboxy-X-rhodamine, triethylammonium salt) moiety, BO-PRO™-3-DNA moiety, BOPRO™-3 moiety, BOBO™-3-DNA moiety, Ethidium Bromide moiety, ReAsH moiety, Calcium Crimson moiety, mRFP moiety, mCherry moiety, HcRed moiety, DyLight™ 594 moiety, Ethidium homodimer-1-DNA moiety, Ethidiumhomodimer moiety, Propidium Iodide moiety, SYPRO® Ruby moiety, Propidium Iodide-DNA moiety, Alexa Fluor® 594 moiety, BODIPY® TR-X, SE moiety, BODIPY® TR-X, BODIPY® TR-X phallacidin pH 7.0 moiety, Alexa Fluor®610 R-phycoerythrin streptavidin pH 7.2 moiety, YO-PRO™-3-DNA moiety, Di-8 ANEPPS moiety, Di-8-ANEPPS-lipid moiety, YOYO™-3-DNA moiety, Nile Red moiety, DyLight™ 633 moiety, mPlum moiety, TO-PRO®-3-DNA moiety, DDAO pH 9.0 moiety, Fura Red™ high Ca moiety, Allophycocyanin pH 7.5 moiety, APC (allophycocyanin) moiety, Nile Blue, TOTO™-3-DNA moiety, Cy® 5 moiety, BODIPY® 650/665-X, Alexa Fluor® 647 R-phycoerythrin streptavidin pH 7.2 moiety, DyLight™ 649 moiety, Alexa Fluor® 647 moiety, Fura Red™ Ca²⁺ moiety, ATTO™ 647 moiety, Fura Red™, low Ca moiety, Carboxynaphthofluorescein pH 10.0 moiety, Alexa Fluor® 660 moiety, Cy® 5.5 moiety, Alexa Fluor® 680 moiety, DyLight™ 680 moiety, Alexa Fluor® 700 moiety, FM™ 4-64, 2% CHAPS moiety, or FM™ 4-64 moiety. In embodiments, the detectable moiety is a moiety of 1,1-Diethyl-4,4-carbocyanine iodide, 1,2-Diphenylacetylene, 1,4-Diphenylbutadiene, 1,4-Diphenylbutadiyne, 1,6-Diphenylhexatriene, 1,6-Diphenylhexatriene, 1-anilinonaphthalene-8-sulfonic acid, 2,7-Dichlorofluorescein, 2,5-Diphenyloxazole, 2-Di-1-ASP, 2-dodecylresorufin, 2-Methylbenzoxazole, 3,3-Diethylthiadicarbocyanine iodide, 4-Dimethylamino-4-Nitrostilbene, 5(6)-Carboxyfluorescein, 5(6)-Carboxynaphtofluorescein, 5(6)-Carboxytetramethylrhodamine B, 5-(and-6)-carboxy-2′,7′-dichlorofluorescein, 5-(and-6)-carboxy-2,7-dichlorofluorescein, 5-(N-hexadecanoyl)aminoeosin, 5-(N-hexadecanoyl)aminoeosin, 5-chloromethylfluorescein, 5-FAM, 5-ROX™, 5-TAMRA, 5-TAMRA, 6,8-difluoro-7-hydroxy-4-methylcoumarin, 6,8-difluoro-7-hydroxy-4-methylcoumarin, 6-carboxyrhodamine 6G, 6-HEX, 6-JOE, 6-TET, 7-aminoactinomycin D, 7-Benzylamino-4-Nitrobenz-2-Oxa-1,3-Diazole, 7-Methoxycoumarin-4-Acetic Acid, 8-Benzyloxy-5,7-diphenylquinoline, 8-Benzyloxy-5,7-diphenylquinoline, 9,10-Bis(Phenylethynyl)Anthracene, 9,10-Diphenylanthracene, 9-METHYLCARBAZOLE, (CS)2Ir(μ-Cl)2Ir(CS)2, AAA, Acridine Orange, Acridine Yellow, Adams Apple Red 680, Adirondack Green 520, Alexa Fluor® 350, Alexa Fluor® 405, Alexa Fluor® 430, Alexa Fluor® 480, Alexa Fluor® 488, Alexa Fluor® 488 hydrazide, Alexa Fluor® 500, Alexa Fluor® 514, Alexa Fluor® 532, Alexa Fluor® 546, Alexa Fluor®555, Alexa Fluor® 568, Alexa Fluor® 594, Alexa Fluor® 610, Alexa Fluor® 610-R-PE, Alexa Fluor® 633, Alexa Fluor® 635, Alexa Fluor® 647, Alexa Fluor® 647-R-PE, Alexa Fluor®660, Alexa Fluor® 680, Alexa Fluor® 680-APC, Alexa Fluor® 680-R-PE, Alexa Fluor®700, Alexa Fluor® 750, Alexa Fluor® 790, Allophycocyanin, AmCyan1, Aminomethylcoumarin, Amplex Gold (product), Amplex Red Reagent, Amplex UltraRed, Anthracene, APC, APC-Seta-750, AsRed2, ATTO™ 390, ATTO™ 425, ATTO™ 430LS, ATTO™ 465, ATTO™ 488, ATTO™ 490LS, ATTO™ 495, ATTO™ 514, ATTO™ 520, ATTO™ 532, ATTO™ 550, ATTO™ 565, ATTO™ 590, ATTO™ 594, ATTO™ 610, ATTO™ 620, ATTO™ 633, ATTO™ 635, ATTO™ 647, ATTO™ 647N, ATTO™ 655, ATTO™ 665, ATTO™ 680, ATTO™ 700, ATTO™ 725, ATTO™ 740, ATTO™ Oxa12, ATTO™ Rho3B, ATTO™ Rho6G, ATTO™ Rho 11, ATTO™ Rho12, ATTO™ Rho13, ATTO™ Rho14, ATTO™ Rho101, ATTO™ Thio12, Auramine O, Azami Green, Azami Green monomeric, B-phycoerythrin, BCECF, BCECF, Bex1, Biphenyl, Birch Yellow 580, Blue-green algae, BO-PRO™-1, BO-PRO™-3, BOBO™-1, BOBO™-3, BODIPY® 630 650-X, BODIPY® 650/665-X, BODIPY® FL, BODIPY® FL, BODIPY® R6G, BODIPY® TMR-X, BODIPY® TR-X, BODIPY® TR-X Ph 7.0, BODIPY® TR-X phallacidin, BODIPY®-DiMe, BODIPY®-Phenyl, BODIPY®-TMSCC, C3-Indocyanine, C3-Oxacyanine, C3-Thiacyanine Dye (EtOH), C3-Thiacyanine Dye (PrOH), C5-Indocyanine, C5-Oxacyanine, C5-Thiacyanine, C7-Indocyanine, C7-Oxacyanine, C545T, C-Phycocyanin, Calcein red-orange, Calcium Crimson, Calcium Green-1, Calcium Orange, Calcofluor white 2MR, Carboxy SNARF-1 pH 6.0, Carboxy SNARF-1 pH 9.0, Carboxynaphthofluorescein, Cascade Blue®, Cascade Yellow, Catskill Green 540, CBQCA, CellMask™ Orange, CellTrace™ BODIPY® TR methyl ester, CellTrace™ calcein violet, CellTrace™ Far Red, CellTracker™ Blue, CellTracker™ Red CMTPX, CellTracker™ Violet BMQC, CF405M, CF405S, CF488A, CF543, CF555, CFP, CFSE, CF™ 350, CF™ 485, Chlorophyll A, Chlorophyll B, Chromeo™ 488, Chromeo™ 494, Chromeo™ 505, Chromeo™ 546, Chromeo™ 642, Citrine, ClOH butoxy aza-BODIPY®, ClOH C12 aza-BODIPY®, CM-H2DCFDA, Coumarin 1, Coumarin 6, Coumarin 30, Coumarin 314, Coumarin 334, Coumarin 343, Coumarine 545T, Cresyl Violet Perchlorate, CryptoLight CF1, CryptoLight CF2, CryptoLight CF3, CryptoLight CF4, CryptoLight CF5, CryptoLight CF6, Crystal Violet, Cumarin153, Cy2®, Cy3®, Cy3.5®, Cy3B®, Cy5® ET, Cy5®, Cy5.5®, Cy7®, Cyanine3 NHS ester, Cyanine5 carboxylic acid, Cyanine5 NHS ester, CypHer5, CypHer5 pH 9.15, CyQUANT™ GR, CyTrak Orange™, Dabcyl SE, DAF-FM™, DAMC (Weiss), dansyl cadaverine, Dansyl Glycine (Dioxane), Dapoxyl (2-aminoethyl)sulfonamide, DDAO, Deep Purple, di-8-ANEPPS, DiA, Dichlorotris(1,10-phenanthroline) ruthenium(II), DiClOH C12 aza-BODIPY®, DiClOHbutoxy aza-BODIPY®, DiD, DiI, DiIC18(3), DiO, DiR, Diversa Cyan-FP, Diversa Green-FP, DM-NERF pH 4.0, DOCI, Doxorubicin, DPP pH-Probe 590-7.5, DPP pH-Probe 590-9.0, DPP pH-Probe 590-11.0, DPP pH-Probe 590-11.0, Dragon Green, DRAQ5™ DsRed, DsRed-Express, DsRed-Express2, DsRed-Express T1, dTomato, DY-350XL, DY-480, DY-480XL MegaStokes, DY-485, DY-485XL MegaStokes, DY-490, DY-490XL MegaStokes, DY-500, DY-500XL MegaStokes, DY-520, DY-520XL MegaStokes, DY-547, DY-549P1, DY-549P1, DY-554, DY-555, DY-557, DY-590, DY-615, DY-630, DY-631, DY-633, DY-635, DY-636, DY-647, DY-649P1, DY-650, DY-651, DY-656, DY-673, DY-675, DY-676, DY-680, DY-681, DY-700, DY-701, DY-730, DY-731, DY-750, DY-751, DY-776, DY-782, Dye-28, Dye-33, Dye-45, Dye-304, Dye-1041, DyLight™ 488, DyLight™ 549, DyLight™ 594, DyLight™ 633, DyLight™ 649, DyLight™ 680, E2-Crimson, E2-Orange, E2-Red/Green, EBFP, ECF, ECFP, ECL Plus, eGFP, ELF 97, Emerald, Envy Green, Eosin, Eosin Y, epicocconone, EqFP611, Erythrosin-5-isothiocyanate, Ethidium bromide, ethidium homodimer-1, Ethyl Eosin, Ethyl Nile Blue A, Ethyl-p-Dimethylaminobenzoate, Eu2O3 nanoparticles, Eu (Soini), Eu(tta)3DEADIT, EvaGreen®, EVOblue®-30, EYFP, FAD, FITC, FITC, FlAsH (Adams), Flash Red EX, FlAsH-CCPGCC, FlAsH-CCXXCC, Fluo-3, Fluo-4, Fluo-5F, Fluorescein-Dibase, fluoro-emerald, Fluorol 5G, FluoSpheres™ blue, FluoSpheres™ crimson, FluoSpheres™ dark red, FluoSpheres™ orange, FluoSpheres™ red, FluoSpheres™ yellow-green, FM™4-64 in CTC, FM™4-64 in SDS, FM™ 1-43, FM™ 4-64, Fort Orange 600, Fura Red™, Fura Red™ Ca free, fura-2, Fura-2 Ca²⁺ free, Gadodiamide, Gd-Dtpa-Bma, GelGreen™, GelRed™, H9-40, HcRed1, Hemo Red 720, HiLyte™ Fluor 488, HiLyte™ Fluor 555, HiLyte™ Fluor 647, HiLyte™ Fluor 680, HiLyte™ Fluor 750, HiLyte™ Plus 555, HiLyte™ Plus 647, HiLyte™ Plus 750, HmGFP, Hoechst 33258, Hoechst 33342, Hoechst-33258, Hops Yellow 560, HPTS, indo-1, Indo-1 Ca free, Ir(Cn)2(acac), Ir(Cs)2(acac), IR-775 chloride, IR-806, Ir—OEP—CO—Cl, IRDye® 650 Alkyne, IRDye® 650 Azide, IRDye® 650 Carboxylate, IRDye® 650 DBCO, IRDye® 650 Maleimide, IRDye® 650 NHS Ester, IRDye® 680LT Carboxylate, IRDye® 680LT Maleimide, IRDye® 680LT NHS Ester, IRDye® 680RD Alkyne, IRDye® 680RD Azide, IRDye® 680RD Carboxylate, IRDye® 680RD DBCO, IRDye® 680RD Maleimide, IRDye® 680RD NHS Ester, IRDye® 700 phosphoramidite, IRDye® 700DX, IRDye® 700DX, IRDye® 700DX Carboxylate, IRDye® 700DX NHS Ester, IRDye® 750 Carboxylate, IRDye® 750 Maleimide, IRDye® 750 NHS Ester, IRDye® 800 phosphoramidite, IRDye® 800CW, IRDye® 800CW Alkyne, IRDye® 800CW Azide, IRDye® 800CW Carboxylate, IRDye® 800CW DBCO, IRDye® 800CW Maleimide, IRDye® 800CW NHS Ester, IRDye® 800RS, IRDye® 800RS Carboxylate, IRDye® 800RS NHS Ester, IRDye® QC-1 Carboxylate, IRDye® QC-1 NHS Ester, JC-1, JOJO™-1, Jonamac Red Evitag T2, Kaede Green, Kaede Red, kusabira orange, Lake Placid 490, LDS 751, Lissamine Rhodamine (Weiss), LOLO™-1, Lucifer Yellow CH, Lucifer Yellow CH Dilitium salt, Lumio Green, Lumio Red, Lumogen F Orange, Lumogen Red F300, LysoSensor® Blue DND-192, LysoSensor® Green DND-153, LysoSensor® Yellow/Blue DND-160 pH 3, LysoSensor® YellowBlue DND-160, LysoTracker® Blue DND-22, LysoTracker® Blue DND-22, LysoTracker® Green DND-26, LysoTracker® Red DND-99, LysoTracker® Yellow HCK-123, Macoun Red Evitag T2, Macrolex® Fluorescence Red G, Macrolex® Fluorescence Yellow 1OGN, Macrolex® Fluorescence Yellow 1OGN, Magnesium Green, Magnesium Octaethylporphyrin, Magnesium Orange, Magnesium Phthalocyanine, Magnesium Phthalocyanine, Magnesium Tetramesitylporphyrin, Magnesium Tetraphenylporphyrin, malachite green isothiocyanate, Maple Red-Orange 620, Marina Blue®, mBanana, mBBr, mCherry, Merocyanine 540, Methyl green, Methylene Blue, mHoneyDew, MitoTracker™ Deep Red 633, MitoTracker™ Green FM™, MitoTracker™ Orange CMTMRos, MitoTracker™ Red CMXRos, monobromobimane, Monochlorobimane, Monoraphidium, mOrange, mOrange2, mPlum, mRaspberry, mRFP, mRFP1, mRFP1.2 (Wang), mStrawberry (Shaner), mTangerine (Shaner), N,N-Bis(2,4,6-trimethylphenyl)-3,4:9,10-perylenebis(dicarboximide), NADH, Naphthalene, Naphthofluorescein, NBD-X, NeuroTrace™ 500525, Nilblau perchlorate, Nile Blue, Nile Red, Nileblue A, NIR1, NIR2, NIR3, NIR4, NIR820, Octaethylporphyrin, OH butoxy aza-BODIPY®, OHC12 aza-BODIPY®, Orange Fluorescent Protein, Oregon Green™ 488, Oregon Green™ 488 DHPE, Oregon Green™ 514, Oxazin1, Oxazin 750, Oxazine 1, Oxazine 170, P4-3, P-Quaterphenyl, P-Terphenyl, PA-GFP (post-activation), PA-GFP (pre-activation), Pacific Orange®, Palladium(II) meso-tetraphenyl-tetrabenzoporphyrin, PdOEPK, PdTFPP, PerCP-Cy5.5®, Perylene, Perylene bisimide pH-Probe 550-5.0, Perylene bisimide pH-Probe 550-5.5, Perylene bisimide pH-Probe 550-6.5, Perylene Green pH-Probe 720-5.5, Perylene Green Tag pH-Probe 720-6.0, Perylene Orange pH-Probe 550-2.0, Perylene Orange Tag 550, Perylene Red pH-Probe 600-5.5, Perylene diimide, Perylne Green pH-Probe 740-5.5, Phenol, Phenylalanine, pHrodo™, succinimidyl ester, Phthalocyanine, PicoGreen® dsDNA quantitation reagent, Pinacyanol-Iodide, Piroxicam, Platinum(II) tetraphenyltetrabenzoporphyrin, Plum Purple, PO-PRO™-1, PO-PRO™-3, POPO™_1, POPO™-3, POPOP, Porphin, PPO, Proflavin, PromoFluor-350, PromoFluor-405, PromoFluor-415, PromoFluor-488, PromoFluor-488LSS, PromoFluor-500LSS, PromoFluor-505, PromoFluor-510LSS, PromoFluor-514LSS, PromoFluor-520LSS, PromoFluor-532, PromoFluor-546, PromoFluor-555, PromoFluor-590, PromoFluor-610, PromoFluor-633, PromoFluor-647, PromoFluor-670, PromoFluor-680, PromoFluor-700, PromoFluor-750, PromoFluor-770, PromoFluor-780, PromoFluor-840, propidium iodide, Protoporphyrin IX, PTIR475/UF, PTIR545/UF, PtOEP, PtOEPK, PtTFPP, Pyrene, QD525, QD565, QD585, QD605, QD655, QD705, QD800, QD903, QD PbS 950, QDot™ 525, QDot™ 545, QDot™ 565, QDot™ 585, QDot™ 605, QDot™ 625, QDot™ 655, QDot™ 705, QDot™ 800, QpyMe2, QSY™ 7, QSY™ 7, QSY™ 9, QSY™ 21, QSY™ 35, quinine, Quinine Sulfate, R-phycoerythrin, ReAsH-CCPGCC, ReAsH-CCXXCC, Red Beads (Weiss), Redmond Red, Resorufin, rhod-2, Rhodamin 700 perchlorate, rhodamine, Rhodamine 6G, Rhodamine 101, rhodamine 110, Rhodamine 123, Rhodamine B, Rhodamine Green, Rhodamine pH-Probe 585-7.0, Rhodamine pH-Probe 585-7.5, Rhodamine phalloidin, Rhodamine Red-X, Rhodamine Tag pH-Probe 585-7.0, Rhodol Green, Riboflavin, Rose Bengal, Sapphire, SBFI, SBFI Zero Na, SensiLight PBXL-1, SensiLight PBXL-3, Seta 633-NHS, Seta-633-NHS, SeTau-380-NHS, SeTau-647-NHS, Snake-Eye Red 900, SNIR1, SNIR2, SNIR3, SNIR4, Sodium Green, Solophenyl flavine 7GFE 500, SpectrumAqua™, Spectrum Blue, Spectrum FRed, Spectrum Gold, Spectrum Green, Spectrum Orange, Spectrum Red, Squarylium dye III, Stains All, Stilbene, Sulfo-Cyanine3 carboxylic acid, Sulfo-Cyanine3 NHS ester, Sulfo-Cyanine5 carboxylic acid, Sulforhodamine 101, Sulforhodamine B, Sulforhodamine G, Suncoast Yellow, SuperGlo BFP, SuperGlo GFP, Surf Green EX, SYBR® Gold nucleic acid gel stain, SYBR® Green I, SYPRO® Ruby, SYTO™ 9, SYTO™ 11, SYTO™ 13, SYTO™ 16, SYTO™ 17, SYTO™ 45, SYTO™ 59, SYTO™ 60, SYTO™ 61, SYTO™ 62, SYTO™ 82, SYTO™ RNASelect, SYTO™ RNASelect, SYTOX™ Blue, SYTOX™ Green, SYTOX™ Orange, SYTOX™ Red, T-Sapphire, Tb (Soini), tCO, tdTomato, Terrylene, Terrylendiimide, Tetra-t-Butylazaporphine, Tetra-t-Butylnaphthalocyanine, Tetracene, Tetrakis(o-Aminophenyl)Porphyrin, Tetramesitylporphyrin, Tetramethylrhodamine, Tetraphenylporphyrin, Texas Red™, Texas Red™ DUPE, Texas Red™-X, ThiolTracker Violet, Thionin acetate, TMRE, TO-PRO®-1, TO-PRO®-3, Toluene, Topaz (Tsien1998), TOTO™-1, TOTO™-3, Tris(2,2-Bipyridyl)Ruthenium(II) chloride, Tris(4,4-diphenyl-2,2-bipyridine) ruthenium(II) chloride, Tris(4,7-diphenyl-1,10-phenanthroline) ruthenium(II) TMS, TRITC Dextran, Tryptophan, Tyrosine, Vex1, Vybrant™ DyeCycle™ Green stain, Vybrant™ DyeCycle™ Orange stain, Vybrant™ DyeCycle™ Violet stain, WEGFP, WellRED D2, WellRED D3, WellRED D4, WtGFP, X-rhod-1, Yakima Yellow, YFP, YO-PRO™-1, YO-PRO™-3, YOYO™-1, YOYO™-1, YOYO™-3, Zinc Octaethylporphyrin, Zinc Phthalocyanine, Zinc Tetramesitylporphyrin, Zinc Tetraphenylporphyrin, ZsGreen1, or ZsYellow1. In embodiments, R⁴ is a monovalent moiety of one of the detectable moieties described immediately above. Janelia Fluor® is a registered trademark of Howard Hughes Medical Institute. Cascade Blue®, SYPRO®, and Oregon Green® are registered trademarks of Life Technologies. LysoTracker™ FluoSpheres™, FM™, Fura Red™, LysoSensor®, SYBR®, TO-PRO®, TOTO™, and Marina Blue® are trademarks of Invitrogen. Pacific Blue™, PO-PRO®, POPO®, SYTOX Blue™, BO-PRO™, BOBO™, YO-PRO™, YOYO™, MitoTracker™, PicoGreen®, NeuroTrace™, Fura Red™, CellTrace™, CellMask™, LOLO™-1, JOJO™-1, Qdot™, QSY™, CyQUANT™ DyLight® dyes, SYTO™, SYTOX Blue™, and Vybrant™ DyeCycle™ are trademarks of Thermo Fisher. BODIPY® is a registered trademark of Molecular Probes. TAMRA™ is a trademark of Appelera. Chromeo™ is a trademark of Active Motif Chromeon GmbH. CyTRACK Orange™ and DRAQ5™ are trademarks of Biostatus Limited. EvaGreen®, GelGreen®, GelRed®, CF®, and FM™ are trademarks of Biotium. Macrolex® is a trademark of Lanxess. SpectrumFRed™, SpectrumRed™, SpectrumGold™, SpectrumOrange™ SpectrumGreen™, SpectrumAqua™, and SpectrumBlue™ Series Vysis′™ SpectrumFRed™ SpectrumRed™, SpectrumGold™, SpectrumOrange™, SpectrumGreen™, SpectrumAqua™ and SpectrumBlue™ are trademarks of Abbott Molecular Inc. HiLyte™ is a trademark of Anaspec, Inc. IRDye® is a trademark of Li-Cor Biosciences, Inc. Rox™ is a trademark of Applied Biosystems. Atto™ is a trademark of ATTO-TEC GmbH. Cy® is a registered trademark of Cytiva.

In embodiments, R⁴ is a fluorescent moiety that has a maximum excitation wavelength between 350-400 nm. In embodiments, R⁴ is a fluorescent moiety that has a maximum excitation wavelength between 400-450 nm. In embodiments, R⁴ is a fluorescent moiety that has a maximum excitation wavelength between 450-500 nm. In embodiments, R⁴ is a fluorescent moiety that has a maximum excitation wavelength between 500-550 nm. In embodiments, R⁴ is a fluorescent moiety that has a maximum excitation wavelength between 550-600 nm. In embodiments, R⁴ is a fluorescent moiety that has a maximum excitation wavelength between 600-650 nm. In embodiments, R⁴ is a fluorescent moiety that has a maximum excitation wavelength between 650-700 nm. In embodiments, R⁴ is a fluorescent moiety that has a maximum excitation wavelength between 700-750 nm. In embodiments, R⁴ is a fluorescent moiety that has a maximum excitation wavelength between 750-800 nm. In embodiments, R⁴ is a fluorescent moiety that has a maximum excitation wavelength of 325 nm, 343 nm, 350 nm, 353 nm, 359 nm, 360 nm, 395 nm, 400 nm, 401 nm, 402 nm, 403 nm, 425 nm, 434 nm, 440 nm, 466 nm, 480 nm, 485 nm, 489 nm, 490 nm, 492 nm, 493 nm, 494 nm, 495 nm, 496 nm, 498 nm, 499 nm, 500 nm, 502 nm, 503 nm, 505 nm, 517 nm, 518 nm, 520 nm, 525 nm, 528 nm, 530 nm, 531 nm, 535 nm, 542 nm, 544 nm, 547 nm, 550 nm, 553 nm, 554 nm, 558 nm, 560 nm, 561 nm, 562 nm, 565 nm, 567 nm, 570 nm, 572 nm, 579 nm, 581 nm, 589 nm, 590 nm, 591 nm, 593 nm, 596 nm, 610 nm, 631 nm, 632 nm, 638 nm, 650 nm, 652 nm, 654 nm, 663 nm, 675 nm, 680 nm, 692 nm, 696 nm, 743 nm, 752 nm, 777 nm, or 782 nm.

In embodiments, R⁴ is a fluorescent moiety that has a maximum emission wavelength between 400-450 nm. In embodiments, R⁴ is a fluorescent moiety that has a maximum emission wavelength between 450-500 nm. In embodiments, R⁴ is a fluorescent moiety that has a maximum emission wavelength between 500-550 nm. In embodiments, R⁴ is a fluorescent moiety that has a maximum emission wavelength between 550-600 nm. In embodiments, R⁴ is a fluorescent moiety that has a maximum emission wavelength between 600-650 nm. In embodiments, R⁴ is a fluorescent moiety that has a maximum emission wavelength between 650-700 nm. In embodiments, R⁴ is a fluorescent moiety that has a maximum emission wavelength between 700-750 nm. In embodiments, R⁴ is a fluorescent moiety that has a maximum emission wavelength between 750-800 nm. In embodiments, R⁴ is a fluorescent moiety that has a maximum emission wavelength between 800-850 nm. In embodiments, R⁴ is a fluorescent moiety that has a maximum emission of 410 nm, 420 nm, 421 nm, 423 nm, 432 nm, 442 nm, 445 nm, 455 nm, 506 nm, 512 nm, 514 nm, 517 nm, 518 nm, 519 nm, 520 nm, 521 nm, 523 nm, 525 nm, 528 nm, 533 nm, 537 nm, 539 nm, 540 nm, 542 nm, 548 nm, 550 nm, 551 nm, 554 nm, 555 nm, 556 nm, 565 nm, 568 nm, 570 nm, 572 nm, 573 nm, 574 nm, 575 nm, 576 nm, 578 nm, 580 nm, 590 nm, 591 nm, 594 nm, 595 nm, 596 nm, 603 nm, 605 nm, 613 nm, 615 nm, 617 nm, 618 nm, 619 nm, 620 nm, 629 nm, 630 nm, 640 nm, 647 nm, 648 nm, 658 nm, 660 nm, 668 nm, 670 nm, 673 nm, 675 nm, 691 nm, 694 nm, 695 nm, 702 nm, 712 nm, 719 nm, 767 nm, 776 nm, 778 nm, 794 nm, or 804 nm.

In embodiments, the first nucleotide, the second nucleotide, the third nucleotide, and the fourth nucleotide independently has the formula:

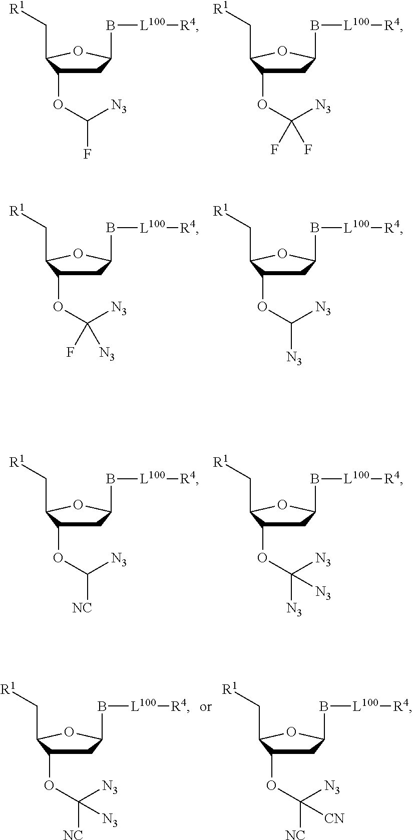

wherein B, L¹⁰⁰, R¹, and R⁴ are as described herein, including embodiments.

In embodiments, the first nucleotide, the second nucleotide, the third nucleotide, and the fourth nucleotide independently has the formula:

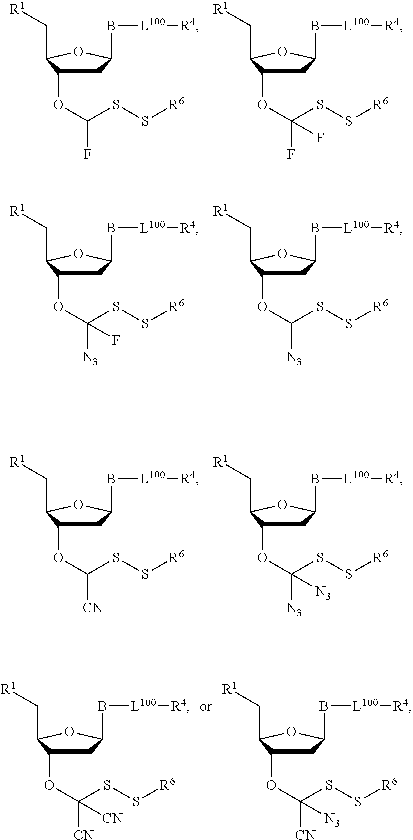

wherein B, L¹⁰⁰, R¹, R⁴, and R⁶ are as described herein, including embodiments.

In embodiments, R⁶ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁶ is substituted or unsubstituted alkyl. In embodiments, R⁶ is substituted alkyl (C₁-C₂₀, C₁₀-C₂₀, C₁-C₈, C₁-C₆, or C₁-C₄). In embodiments, R⁶ is unsubstituted alkyl (C₁-C₂₀, C₁₀-C₂₀, C₁-C₈, C₁-C₆, or C₁-C₄).

In embodiments, R⁶ is unsubstituted C₁-C₄ alkyl. In embodiments, R⁶ is unsubstituted C₁-C₆ alkyl. In embodiments, R⁶ is unsubstituted C₂ alkyl. In embodiments, R⁶ is unsubstituted C₃ alkyl. In embodiments, R⁶ is unsubstituted C₄ alkyl. In embodiments, R⁶ is unsubstituted C₅ alkyl. In embodiments, R⁸ is unsubstituted C₆ alkyl. In embodiments, R⁶ is unsubstituted methyl. In embodiments, R⁶ is unsubstituted ethyl. In embodiments, R⁶ is unsubstituted propyl. In embodiments, R⁶ is unsubstituted isopropyl. In embodiments, R⁶ is unsubstituted butyl. In embodiments, R⁶ is unsubstituted tert-butyl. In embodiments, R⁶ is unsubstituted pentyl. In embodiments, R⁶ is unsubstituted hexyl.

In embodiments, R⁶ is unsubstituted C₁-C₆ or C₁-C₄ saturated alkyl. In embodiments, R⁶ is unsubstituted C₁-C₄ saturated alkyl. In embodiments, R⁶ is unsubstituted C₁-C₆ saturated alkyl. In embodiments, R⁶ is unsubstituted methyl. In embodiments, R⁶ is unsubstituted C₂ saturated alkyl. In embodiments, R⁶ is unsubstituted C₃ saturated alkyl. In embodiments, R⁶ is unsubstituted C₄ saturated alkyl. In embodiments, R⁶ is unsubstituted C₅ saturated alkyl. In embodiments, R⁶ is unsubstituted C₆ saturated alkyl.

In embodiments, R⁶ is substituted C₁-C₄ alkyl. In embodiments, R⁶ is substituted C₁-C₆ alkyl. In embodiments, R⁶ is substituted C₂ alkyl. In embodiments, R⁶ is substituted C₃ alkyl. In embodiments, R⁶ is substituted C₄ alkyl. In embodiments, R⁶ is substituted C₅ alkyl. In embodiments, R⁸ is substituted C₆ alkyl. In embodiments, R⁶ is substituted methyl. In embodiments, R⁶ is substituted ethyl. In embodiments, R⁶ is substituted propyl. In embodiments, R⁶ is substituted isopropyl. In embodiments, R⁶ is substituted butyl. In embodiments, R⁶ is substituted tert-butyl. In embodiments, R⁶ is substituted pentyl. In embodiments, R⁶ is substituted hexyl.

In embodiments, R⁶ is R⁶⁰⁰-substituted or unsubstituted alkyl (e.g., C₁-C₂₀, C₁₀-C₂₀,
″\*MERGEFORMAT\*MERGEFORMAT C₁-C₈, C₁-C₆, or C₁-C₄), R⁶⁰⁰-substituted or unsubstituted heteroalkyl (e.g., 2 to 20, 8 to 20, 2 to 10, 2 to 8, 2 to 6, or 2 to 4 membered), R⁶⁰⁰-substituted or unsubstituted cycloalkyl (e.g., C₃-C₈, C₃-C₆, or C₅-C₆), R⁶⁰⁰-substituted or unsubstituted heterocycloalkyl (e.g., 3 to 8, 3 to 6, or 5 to 6 membered), R⁶⁰⁰-substituted or unsubstituted aryl (e.g., C₆-C₁₀, C₁₀, or phenyl), or
″\*MERGEFORMAT\*MERGEFORMAT R⁶⁰⁰-substituted or unsubstituted heteroaryl (e.g., 5 to 10, 5 to 9, or 5 to 6 membered). In embodiments, R⁶ is substituted or unsubstituted alkyl (e.g., C₁-C₂₀, C₁₀-C₂₀, C₁-C₈, C₁-C₆, or
″\*MERGEFORMAT\*MERGEFORMAT C₁-C₄), substituted or unsubstituted heteroalkyl (e.g., 2 to 20, 8 to 20, 2 to 10, 2 to 8, 2 to 6, or 2 to 4 membered), substituted or unsubstituted cycloalkyl (e.g., C₃-C₈, C₃-C₆, or C₅-C₆), substituted or unsubstituted heterocycloalkyl (e.g., 3 to 8, 3 to 6, or 5 to 6 membered), substituted or unsubstituted aryl (e.g., C₆-C₁₀, C₁₀, or phenyl), or substituted or unsubstituted heteroaryl (e.g., 5 to 10, 5 to 9, or 5 to 6 membered).

R⁶⁰⁰ is oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H, —SO₄H,
″\*MERGEFORMAT\*MERGEFORMAT —SO₂NH₂, —NHNH₂, —ONH₂, —NHC(O)NHNH₂, —NHC(O)NH₂, —NHSO₂H, —NHC(O)H,
″\*MERGEFORMAT\*MERGEFORMAT —NHC(O)OH, —NHOH, —OCCl₃, —OCF₃, —OCBr₃, —OCI₃, —OCHCl₂, —OCHBr₂, —OCHI₂, —OCHF₂,
″\*MERGEFORMAT\*MERGEFORMAT —OCH₂Cl, —OCH₂Br, —OCH₂I, —OCH₂F, —N₃, —SF₅, unsubstituted alkyl (e.g., C₁-C₂₀, C₁₀-C₂₀,
″\*MERGEFORMAT\*MERGEFORMAT C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, the first nucleotide, the second nucleotide, the third nucleotide, and the fourth nucleotide independently has the formula:

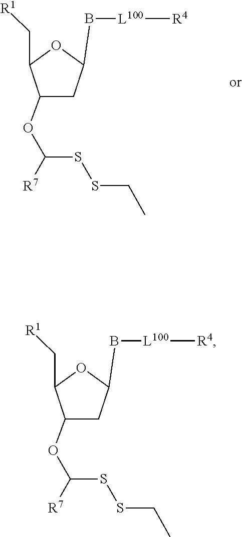

wherein B, R⁷, L¹⁰⁰, and R⁴ are as described herein, including in embodiments. R⁷ is

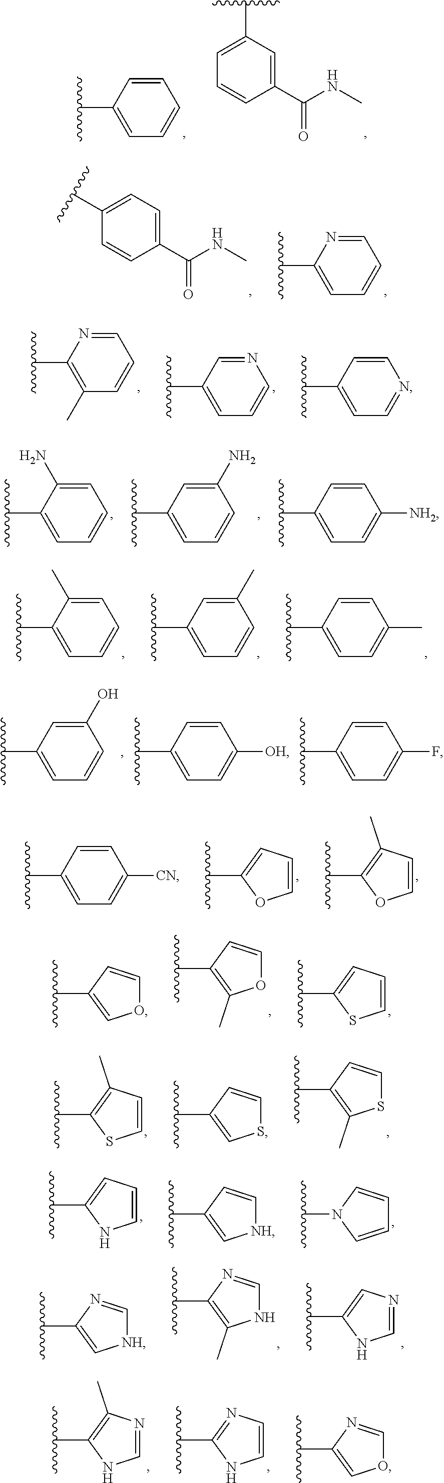

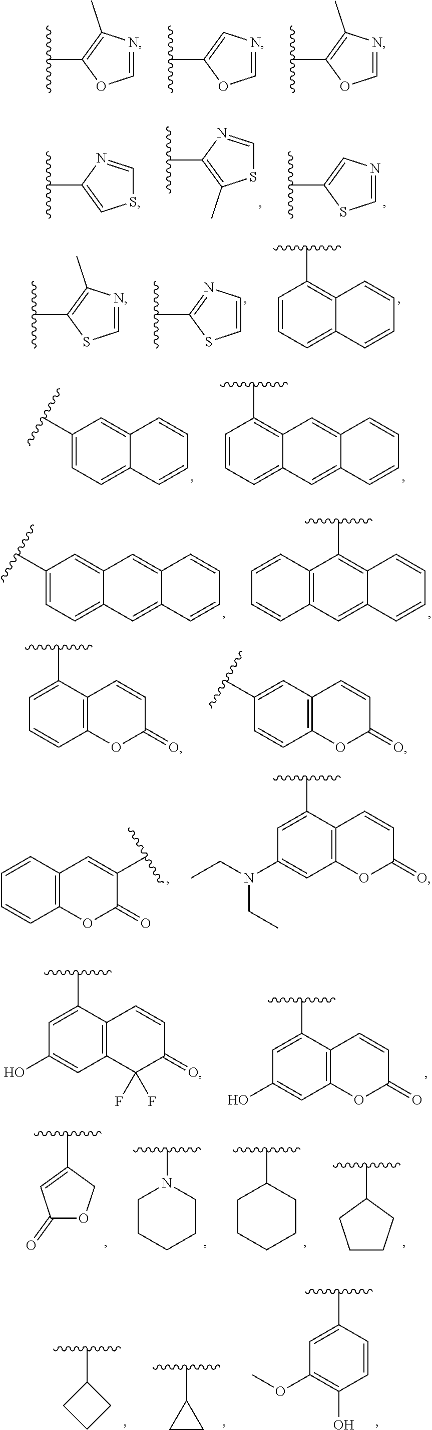

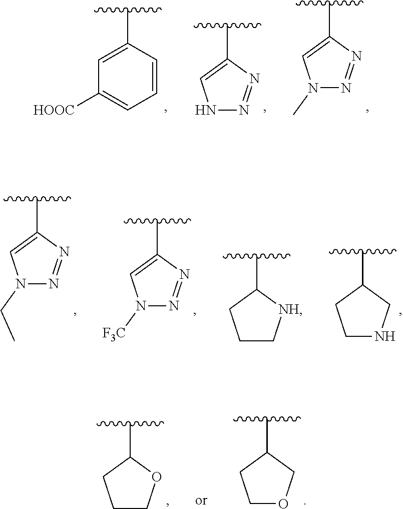

In embodiments, R⁷ is

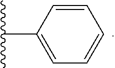 [SMILES: *c1ccccc1]

In embodiments, the first nucleotide, the second nucleotide, the third nucleotide, and the fourth nucleotide independently has the formula:

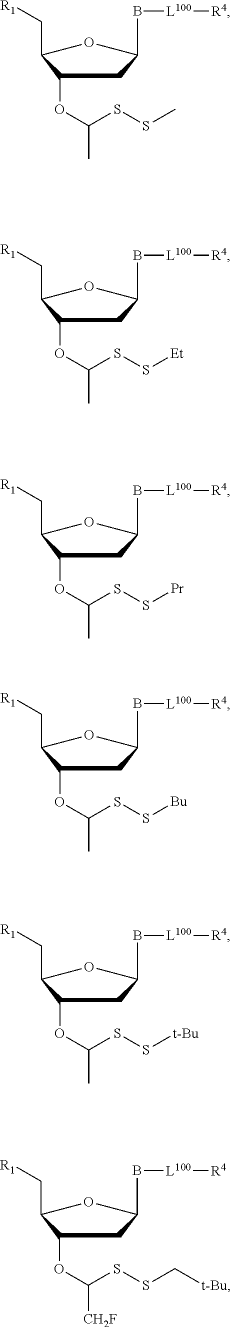

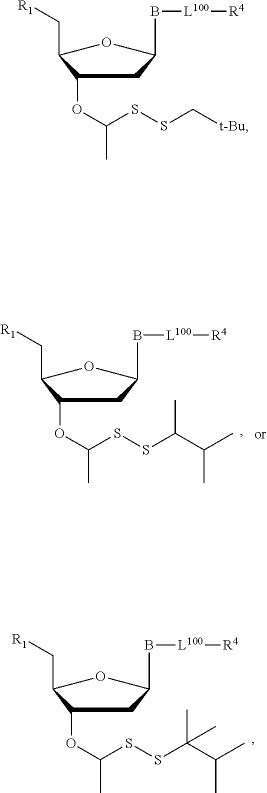

wherein Et, Pr, Bu, and t-Bu are abbreviations for ethyl, propyl, butyl, and tert-butyl moieties.

In an aspect is provided a kit. In embodiments, the kit includes a composition as described herein. In embodiments, the kit includes the reagents and containers useful for performing the methods as described herein. In embodiments, the kit includes a plurality of the first and second probes any one of the aspects and embodiments herein. In embodiments, the kit includes a plurality of the first, second, third, and fourth nucleotides of any one of the aspects and embodiments herein. Generally, the kit includes one or more containers providing a composition and one or more additional reagents (e.g., a buffer suitable for polynucleotide extension and/or sequencing). The kit may also include a template nucleic acid (DNA and/or RNA), one or more primer polynucleotides, one or more nucleotides described herein, and/or nucleoside triphosphates (including, e.g., deoxyribonucleotides, ribonucleotides, labeled nucleotides, and/or modified nucleotides), buffers, salts, and/or labels (e.g., fluorophores). In embodiments, the kit includes a multiwell container, a microplate, and/or reagents for sample preparation and purification, amplification, and/or sequencing (e.g., one or more sequencing reaction mixtures).

In embodiments, the kit includes a solid support. In embodiments, the kit includes a solid support including a cell or tissue immobilized to the surface of the solid support. In embodiments, kit includes a solid support, wherein the solid support includes a functionalized glass surface or a functionalized plastic surface (e.g., a surface including a plurality of reactive moieties).

In embodiments, amplification reagents and other reagents may be provided in lyophilized form. In embodiments, amplification reagents and other reagents may be provided in a container that includes wells within which the lyophilized reagent may be reconstituted.

In embodiments, the kit includes components useful for circularizing template polynucleotides using a ligation enzyme (e.g., Circligase enzyme, Taq DNA Ligase, HiFi Taq DNA Ligase, T4 ligase, SplintR ligase, or Ampligase DNA Ligase). For example, such a kit further includes the following components: (a) reaction buffer for controlling pH and providing an optimized salt composition for a ligation enzyme (e.g., Circligase enzyme, Taq DNA Ligase, HiFi Taq DNA Ligase, T4 ligase, SplintR ligase, or Ampligase DNA Ligase), and (b) ligation enzyme cofactors. In embodiments, the kit further includes instructions for use thereof. In embodiments, kits described herein include a polymerase. In embodiments, the polymerase is a DNA polymerase. In embodiments, the DNA polymerase is a thermophilic nucleic acid polymerase. In embodiments, the DNA polymerase is a modified archaeal DNA polymerase. In embodiments, the kit includes a sequencing solution. In embodiments, the sequencing solution include labeled nucleotides including differently labeled nucleotides, wherein the label (or lack thereof) identifies the type of nucleotide. For example, each adenine nucleotide, or analog thereof; a thymine nucleotide; a cytosine nucleotide, or analog thereof; and a guanine nucleotide, or analog thereof may be labeled with a different fluorescent label. In embodiments, the kit includes a modified terminal deoxynucleotidyl transferase (TdT) enzyme.

In embodiments, the kit further includes a cleaving agent. In embodiments, the kit further includes a first cleaving agent and a second cleaving agent. In embodiments, the cleaving agent is a reducing agent. In embodiments, the cleaving agent is a phosphine containing agent. In embodiments, the cleaving agent is a thiol containing agent. In embodiments, the cleaving agent is di-mercaptopropane sulfonate (DMPS). In embodiments, the cleaving agent is aqueous sodium sulfide (Na₂S). In embodiments, the cleaving agent is Tris-(2-carboxyethyl)phosphines trisodium salt (TCEP), tris(hydroxypropyl)phosphine (THPP), guanidine, urea, cysteine, 2-mercaptoethylamine, or dithiothreitol (DTT). In embodiments, the cleaving agent is an acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄). In embodiments, the method includes contacting the compound (e.g., a compound described herein) with a reducing agent. In embodiments, the kit further includes a wash buffer and an assay buffer.

In embodiments, the kit includes a buffered solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can include one or more divalent cations. Examples of divalent cations can include, but are not limited to, Mg²⁺, Mn²⁺, Zn²⁺, and Ca²⁺. In embodiments, the buffered solution can contain one or more divalent cations at a concentration sufficient to permit hybridization of a nucleic acid. In embodiments, the buffered solution can contain one or more divalent cations at a concentration sufficient to permit hybridization of a nucleic acid. In embodiments, the buffered solution includes about 10 mM Tris, about 20 mM Tris, about 30 mM Tris, about 40 mM Tris, or about 50 mM Tris. In embodiments the buffered solution includes about 50 mM NaCl, about 75 mM NaCl, about 100 mM NaCl, about 125 mM NaCl, about 150 mM NaCl, about 200 mM NaCl, about 300 mM NaCl, about 400 mM NaCl, or about 500 mM NaCl. In embodiments, the buffered solution includes about 0.05 mM EDTA, about 0.1 mM EDTA, about 0.25 mM EDTA, about 0.5 mM EDTA, about 1.0 mM EDTA, about 1.5 mM EDTA or about 2.0 mM EDTA. In embodiments, the buffered solution includes about 0.01% Triton X-100, about 0.025% Triton X-100, about 0.05% Triton X-100, about 0.1% Triton X-100, or about 0.5% Triton X-100. In embodiments, the buffered solution includes 20 mM Tris pH 8.0, 100 mM NaCl, 0.1 mM EDTA, 0.025% Triton X-100. In embodiments, the buffered solution includes 20 mM Tris pH 8.0, 150 mM NaCl, 0.1 mM EDTA, 0.025% Triton X-100. In embodiments, the buffered solution includes a poloxamer. In embodiments, the buffered solution includes about 0.002% Pluronic® F-127, about 0.01% Pluronic® F-127, about 0.02% Pluronic® F-127, about 0.05% Pluronic® F-127, about 0.1% Pluronic® F-127, about 0.2% Pluronic® F-127, about 0.3% Pluronic® F-127, about 0.4% Pluronic® F-127, about 0.5% Pluronic® F-127, about 0.6% Pluronic® F-127, about 0.7% Pluronic® F-127, about 0.8% Pluronic® F-127, about 0.9% Pluronic® F-127, about 1% Pluronic® F-127, about 1.1% Pluronic® F-127, about 1.2% Pluronic® F-127, about 1.3% Pluronic® F-127, about 1.4% Pluronic® F-127, about 1.5% Pluronic® F-127, about 1.6% Pluronic® F-127, about 1.7% Pluronic® F-127, about 1.8% Pluronic® F-127, about 1.9% Pluronic® F-127, or about 2% Pluronic® F-127. In embodiments, the buffered solution includes 0.1 mM DTT, 0.5 mM DTT, 1 mM DTT, 2 mM DTT, 3 mM DTT, 4 mM DTT, 5 mM DTT, 6 mM DTT, 7 mM DTT, 8 mM DTT, 9 mM DTT, 10 mM DTT, 11 mM DTT, 12 mM DTT, 13 mM DTT, 14 mM DTT, 15 mM DTT, 16 mM DTT, 17 mM DTT, 18 mM DTT, 19 mM DTT, or 20 mM DTT. Triton™ is a registered trademark of Dow Chemical Company. In embodiments, the buffered solution includes about 1 mM MgCl₂, about 2 mM MgCl₂, about 3 mM MgCl₂, about 4 mM MgCl₂, about 5 mM MgCl₂, about 6 mM MgCl₂, about 7 mM MgCl₂, about 8 mM MgCl₂, about 9 mM MgCl₂, about 10 mM MgCl₂, about 11 mM MgCl₂, about 12 mM MgCl₂, about 13 mM MgCl₂, about 14 mM MgCl₂, about 15 mM MgCl₂, about 16 mM MgCl₂, about 17 mM MgCl₂, about 18 mM MgCl₂, about 19 mM MgCl₂, or about 20 mM MgCl₂. In embodiments, the buffered solution includes about 0.01 mM ATP, about 0.05 mM ATP, about 0.1 mM ATP, about 0.25 mM ATP, about 0.5 mM ATP, about 0.75 mM ATP, about 1 mM ATP, about 2 mM ATP, about 3 mM ATP, about 4 mM ATP, about 5 mM ATP, about 6 mM ATP, about 7 mM ATP, about 8 mM ATP, about 9 mM ATP, or about 10 mM ATP. In embodiments, the buffered solution includes about 25 mM LiCl, about 50 mM LiCl, about 75 mM LiCl, about 100 mM LiCl, about 125 mM LiCl, about 150 mM LiCl, about 175 mM LiCl, about 200 mM LiCl, about 225 mM LiCl, about 250 mM LiCl, about 275 mM LiCl, about 300 mM LiCl, about 325 mM LiCl, about 350 mM LiCl, about 375 mM LiCl, about 400 mM LiCl, about 425 mM LiCl, about 450 mM LiCl, about 475 mM LiCl, or about 500 mM LiCl. In embodiments, the buffered solution includes 20 mM Tris pH 8.0, 300 mM NaCl, 0.1 mM EDTA, 0.025% Triton X-100. In embodiments, the buffered solution includes 20 mM Tris pH 8.0, 400 mM NaCl, 0.1 mM EDTA, 0.025% Triton X-100. In embodiments, the buffered solution includes 20 mM Tris pH 8.0, 500 mM NaCl, 0.1 mM EDTA, 0.025% Triton X-100.

In embodiments, the kit includes one or more sequencing reaction mixtures. In embodiments, the sequencing reaction mixture includes a buffer. In embodiments, the buffer includes an acetate buffer, 3-(N-morpholino)propanesulfonic acid (MOPS) buffer, N—(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

In embodiments, the kit includes, without limitation, nucleic acid primers, probes, adapters, enzymes, and the like, and are each packaged in a container, such as, without limitation, a vial, tube or bottle, in a package suitable for commercial distribution, such as, without limitation, a box, a sealed pouch, a blister pack and a carton. The package typically contains a label or packaging insert indicating the uses of the packaged materials. As used herein, “packaging materials” includes any article used in the packaging for distribution of reagents in a kit, including without limitation containers, vials, tubes, bottles, pouches, blister packaging, labels, tags, instruction sheets and package inserts.

In addition to the above components, the subject kits may further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, digital storage medium, etc., on which the information has been recorded. Yet another means that may be present is a website address which may be used via the Internet to access the information at a removed site. Any convenient means may be present in the kits.

In embodiments, the kit can further include one or more biological stain(s) (e.g., any of the biological stains as described herein). For example, the kit can further include eosin and hematoxylin. In other examples, the kit can include a biological stain such as acridine orange, Bismarck brown, carmine, coomassie blue, cresyl violet, DAPI, eosin, ethidium bromide, acid fuchsin, hematoxylin, Hoechst stains, iodine, methyl green, methylene blue, neutral red, Nile blue, Nile red, osmium tetroxide, propidium iodide, rhodamine, safranin, or any combination thereof.

In embodiments, the kit further includes a sample collection device. In embodiments, the sample collection device includes EDTA or heparin (e.g., when the sample is obtained from plasma). In embodiments, following collection the sample is stored at less than −20° C. In embodiments, the sample collection device is a serum separator tube (SST). In embodiments, the sample collection device is a vial. In some embodiments, the kit includes instructions for sample collection. In embodiments, the kit includes instructions and information on fasting, diet, and medication restrictions. In embodiments, the kit includes reagents (e.g., ethanol), sterilizing swabs, a marking pen, cotton, distilled water, spoons, scoops, tongue depressor, forceps, tongs, spatula, pipettes, Moore swabs (i.e., gauze strips), sponges, containers, and/or plastic bags. In embodiments, the kit includes an ice pack. In embodiments, the individual components of the kit can be alternatively contained either together in one storage container or separately in two or more storage containers (e.g., separate bottles or vials). In embodiments, the kit includes nucleotides in a buffer. In embodiments, the kit includes a buffer. For example, the sequencing solution and/or the chase solution may include a buffer such as ethanolamine (EA), tris(hydroxymethyl)aminomethane (Tris), glycine, a carbonate salt, a phosphate salt, a borate salt, 2-dimethyalaminomethanol (DMEA), 2-diethyalaminomethanol (DEEA), N,N,N′,N′-tetramethylethylenediamine (TEMED), and N,N,N′,N′-tetraethylethylenediamine (TEEDA), and combinations thereof. For example, the buffer may Tris-HCl (pH 9.2 at 25° C.), ammonium sulfate, MgCl₂, 0.1% Tween® 20, and dNTPs.

In embodiments, the kit is stored for 1 to 90 days. In embodiments, the kit is stored for greater than 90 days. In embodiments, the kit is stored for 1 to 30 days. In embodiments, the kit is stored for 1, 5, 7, 14, 21, 30, 45, 60, 75, 90, or more days. In embodiments, the kit is stored at less than about 25° C. In embodiments, the kit is stored at less than about 5° C. In embodiments, the kit is stored at about 4° C. In embodiments, the kit is stored in the dark (e.g., in the absence of light, such as visible light or UV light). In embodiments, the kit is stored at 2-8° C. In embodiments, the kit is stored for at least 1 day, at least 2 days, at least 3 days, or at least 7 days. In embodiments, the kit is stored for about 1 week, about 2 weeks, about 3 weeks, about 4 weeks, about 5 weeks, about 6 weeks, about 7 weeks, or about 8 weeks. In embodiments, the kit is stored for about 1 month, about 2 months, about 3 months, about 4 months, about 5 months, about 6 months, about 7 months, about 8 months, about 9 months, about 10 months, about 11 months, or about 12 months. In embodiments, the kit is stored at about 2° C. −8° C., about 20° C.-30° C., or about 4° C.-37° C.

### III. Methods

In an aspect is a provided a method of extending a primer. In embodiments, the method described herein includes (a) adding an extension solution comprising four nucleotides to a reaction vessel comprising a polymerase and the primer, wherein the primer is hybridized to a target polynucleotide, and incorporating one of four nucleotides into the primer, wherein the extension solution includes a first nucleotide including a first fluorophore moiety attached to the nucleotide via a first cleavable linker; a second nucleotide including a second fluorophore moiety attached to the nucleotide via a second cleavable linker; a third nucleotide including a third fluorophore moiety attached to the nucleotide via a third cleavable linker; a fourth nucleotide including a fourth fluorophore moiety attached to the nucleotide via a fourth cleavable linker, wherein the first and the second cleavable linkers are cleavable under identical conditions and the third and the fourth cleavable linkers are cleavable under different identical conditions and (b) exciting a fluorophore, wherein exciting includes (i) directing a first excitation light and second excitation light at the reaction vessel; (ii) adding a first cleaving agent into the reaction vessel; followed by (iii) adding a second cleaving agent into the reaction vessel. In embodiments, the method further includes detecting the incorporated nucleotide, wherein detecting includes detecting an emission light after step (i) (i.e., directing a first excitation light and second excitation light at the reaction vessel). In embodiments, the method further includes detecting the incorporated nucleotide, wherein detecting comprises detecting a first emission light after step (i) (i.e., directing a first excitation light and second excitation light at the reaction vessel) and detecting a second emission light after step (ii) (i.e., adding a first cleaving agent into the reaction vessel). In embodiments, the method further includes determining the identity of the incorporated nucleotide based on the detection of the emission light.

In embodiments, the method further includes determining the identity of the incorporated nucleotide based on the detection of the emission light after step (i) (i.e., directing a first excitation light and second excitation light at the reaction vessel) and detecting the absence of the emission light after step (ii) (i.e., adding a first cleaving agent into the reaction vessel). In embodiments, the method further includes determining the identity of the incorporated nucleotide based on the detection of the emission light after step (i) (i.e., directing a first excitation light and second excitation light at the reaction vessel) and detecting the absence of the emission light after step (iii) (i.e., adding a second cleaving agent into the reaction vessel). In embodiments, the method further includes determining the identity of the incorporated nucleotide based on the detection of the emission light after step (i) (i.e., directing a first excitation light and second excitation light at the reaction vessel) and detecting the absence of the emission light after step (ii) (i.e., adding a first cleaving agent into the reaction vessel) and step (iii) (i.e., adding a second cleaving agent into the reaction vessel). In embodiments, the method further includes determining the identity of the incorporated nucleotide based on the detection of the emission light after directing a first excitation light and second excitation light at the reaction vessel and detecting the absence of the emission light following addition of a cleaving agent. In embodiments, the method further includes determining the identity of the incorporated nucleotide based on the detection of the first emission light and the second emission light.

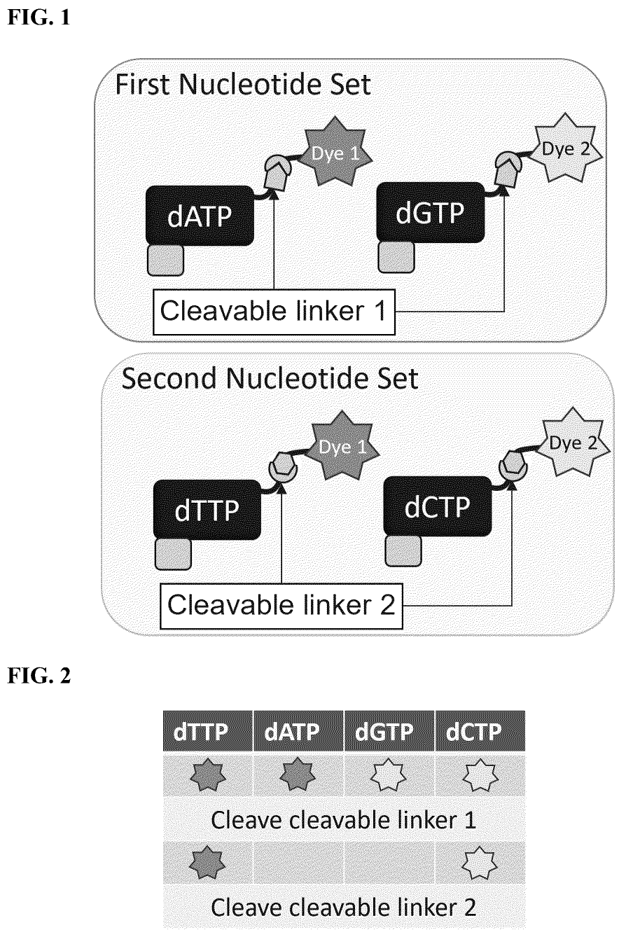

In embodiments, determining the identity of the incorporated nucleotide includes recording the signal derived the emission light of a fluorophore moiety to identify the incorporated nucleotide based on the unique fluorescence emission of each fluorophore moiety conjugated to each fluorescently labelled nucleotide and observing the absence of signal following the addition of a cleaving agent. For example, as shown in FIGS. 1 and 2, it could be determined that an adenine nucleotide is the identity of the incorporated nucleotide during a sequencing cycle if a signal that derived from the emission light of Dye 1 was detected and then disappeared following the addition of a cleaving agent specific for cleavable linker 1. In a different example with FIGS. 1 and 2, it could be determined that a cytosine nucleotide is the identity of the incorporated nucleotide during a sequencing cycle if a signal that derived from the emission light of Dye 2 was detected, and remained following the addition of cleaving agent for cleavable linker 1.

In embodiments, extension solution includes an adenine nucleotide including a first fluorophore moiety attached to the nucleotide via a first cleavable linker; a guanine nucleotide including a second fluorophore moiety attached to the nucleotide via a second cleavable linker; a cytosine nucleotide including a third fluorophore moiety attached to the nucleotide via a third cleavable linker; a thymine nucleotide including a fourth fluorophore moiety attached to the nucleotide via a fourth cleavable linker. In embodiments, the extension solution includes a polymerase. In embodiments, the extension solution includes magnesium (e.g., MgCl₂).

In embodiments, extension solution includes an adenine nucleotide including a first fluorophore moiety attached to the nucleotide via a first cleavable linker; a cytosine nucleotide including a second fluorophore moiety attached to the nucleotide via a second cleavable linker; a guanine nucleotide including a third fluorophore moiety attached to the nucleotide via a third cleavable linker; a thymine nucleotide including a fourth fluorophore moiety attached to the nucleotide via a fourth cleavable linker.

In embodiments, the first cleavable linker and the second cleavable linker are selected from the group: an enzyme-cleavable linker, a photocleavable linker, an acid-cleavable linker, a base-cleavable linker, an oxidant-cleavable linker, a reductant-cleavable linker, or a fluoride-cleavable linker; and the third cleavable linker and the fourth cleavable linker are selected from the group: an enzyme-cleavable linker, a photocleavable linker, an acid-cleavable linker, a base-cleavable linker, an oxidant-cleavable linker, a reductant-cleavable linker, or a fluoride-cleavable linker. In embodiments, the first cleavable linker and the second cleavable linker include a disulfide moiety, a dialkylketal moiety, an allyl moiety, an azide moiety, a hydrazine moiety, a cyanoethyl moiety, or a nitrobenzyl moiety. In embodiments, cleavable linker includes a polynucleotide sequence including a restriction site. In embodiments, cleavable linker includes a restriction site. In embodiments, the first cleavable linker and second cleavable linker includes a restriction site. In embodiments, the third cleavable linker and fourth cleavable linker includes a restriction site. In embodiments, the first cleavable linker and the second cleavable linker are orthogonal cleavable linkers relative to the third cleavable linker and fourth cleavable linker. In embodiments, the first cleavable linker and the second cleavable linker includes reductant-cleavable moiety, and the third cleavable linker and the fourth cleavable linker include an oxidant-cleavable moiety. In embodiments, the first cleavable linker and the second cleavable linker includes reductant-cleavable moiety, and the third cleavable linker and the fourth cleavable linker include an enzyme-cleavable moiety. In embodiments, the first cleavable linker and the second cleavable linker includes a disulfide moiety, and the third cleavable linker and the fourth cleavable linker include a β-galactosidase substrate moiety. In embodiments, the first cleavable linker and the second cleavable linker includes a disulfide moiety, and the third cleavable linker and the fourth cleavable linker include a vicinal (i.e., 1,2-diol) diol moiety.

In embodiments, the first cleavable linker includes an enzyme-cleavable linker, a photocleavable linker, an acid-cleavable linker, a base-cleavable linker, an oxidant-cleavable linker, a reductant-cleavable linker, or a fluoride-cleavable linker. In embodiments, the first cleavable linker includes a restriction site. In embodiments, the first cleavable linker includes a disulfide moiety, a dialkylketal moiety, an allyl moiety, an azide moiety, a hydrazine moiety, a cyanoethyl moiety, or a nitrobenzyl moiety.

In embodiments, the second cleavable linker includes an enzyme-cleavable linker, a photocleavable linker, an acid-cleavable linker, a base-cleavable linker, an oxidant-cleavable linker, a reductant-cleavable linker, or a fluoride-cleavable linker. In embodiments, the second cleavable linker includes a restriction site. In embodiments, the second cleavable linker includes a disulfide moiety, a dialkylketal moiety, an allyl moiety, an azide moiety, a hydrazine moiety, a cyanoethyl moiety, or a nitrobenzyl moiety.

In embodiments, the third cleavable linker includes an enzyme-cleavable linker, a photocleavable linker, an acid-cleavable linker, a base-cleavable linker, an oxidant-cleavable linker, a reductant-cleavable linker, or a fluoride-cleavable linker. In embodiments, the third cleavable linker includes a restriction site. In embodiments, the third cleavable linker includes a disulfide moiety, a dialkylketal moiety, an allyl moiety, an azide moiety, a hydrazine moiety, a cyanoethyl moiety, or a nitrobenzyl moiety.

In embodiments, the fourth cleavable linker includes an enzyme-cleavable linker, a photocleavable linker, an acid-cleavable linker, a base-cleavable linker, an oxidant-cleavable linker, a reductant-cleavable linker, or a fluoride-cleavable linker. In embodiments, the fourth cleavable linker includes a restriction site. In embodiments, the fourth cleavable linker includes a disulfide moiety, a dialkylketal moiety, an allyl moiety, an azide moiety, a hydrazine moiety, a cyanoethyl moiety, or a nitrobenzyl moiety.

In embodiments, the method includes contacting the cleavable linker as described herein (e.g., the first cleavable linker as described herein, the second cleavable linker as described herein, the third cleavable linker as described herein, and/or the fourth cleavable linker as described herein) with a cleaving agent. In embodiments, adding a cleaving agent into the reaction vessel includes contacting the cleavable linker with a cleaving agent, cleaving the first and second cleavable linkers, while leaving the other cleavable linkers (i.e., the third and the fourth) unmodified. In embodiments, the first cleavable linker and second cleavable linker are cleaved, while the third cleavable linker and fourth cleavable linker remain (i.e., remain uncleaved) following the exposure to the first cleaving agent. In embodiments, the third cleavable linker and fourth cleavable linker are cleaved following the exposure to the second cleaving agent. In embodiments, the first cleavable linker and second cleavable linker are cleaved under identical conditions. In embodiments, the third cleavable linker and fourth cleavable linker are cleaved under identical conditions. In embodiments, the first cleavable linker and second cleavable linker are cleaved under orthogonal cleaving conditions relative to the cleaving conditions specific for third cleavable linker and fourth cleavable linker.

In embodiments, the cleaving agent cleaves the cleavable site of cleavable linker as described herein (e.g., the first cleavable linker as described herein, the second cleavable linker as described herein, the third cleavable linker as described herein, and/or the fourth cleavable linker as described herein). In embodiments, the cleavable linker is cleaved by contacting the cleavable linker with a cleaving agent. In embodiments, the cleavable linker can be cleaved by enzymes, nucleophilic/basic reagents, reducing agents, photo-irradiation, electrophilic/acidic reagents, organometallic and metal reagents, or oxidizing reagents. In embodiments, the cleavable linker can be chemically cleaved by a chemical. In embodiments, the chemically cleavable linker is split in response to the presence of a acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄). In embodiments, the cleaving agent is a phosphine containing reagent (e.g., TCEP or THPP), sodium dithionite (Na₂S₂O₄), weak acid, hydrazine (N₂H₄), Pd(0), or light-irradiation (e.g., ultraviolet radiation). In embodiments, a chemically cleavable linker is non-enzymatically cleavable. In embodiments, cleaving includes removing. In embodiments, the cleavable linker includes one or more cleavable site(s). Any suitable enzymatic, chemical, or photochemical cleavage reaction may be used to cleave the cleavable site. In embodiments, cleaving the cleavable linker can be chemical cleavage of a bond between a deoxyribonucleotide and a ribonucleotide, in which case, the cleavable site may include one or more ribonucleotides. In embodiments, cleaving of the cleavable site can be a chemical reduction of a disulfide linkage with a reducing agent (e.g., THPP or TCEP), in which case, the cleavable site should include an appropriate disulfide linkage; chemical cleavage of a diol linkage with periodate, in which case, the cleavable site should include a diol linkage; generation of an abasic site and subsequent hydrolysis, etc. In embodiments, the linker includes a diol linkage, which permits cleavage by treatment with periodate (e.g., sodium periodate). It will be appreciated that more than one diol can be included at the cleavable site. One or more diol units may be incorporated into a polynucleotide using standard methods for automated chemical DNA synthesis. The diol linker is cleaved by treatment with any substance which promotes cleavage of the diol (e.g., a diol-cleaving agent). In embodiments, the diol-cleaving agent is periodate, e.g., aqueous sodium periodate (NaIO₄). Following treatment with the diol-cleaving agent (e.g., periodate) to cleave the diol, the cleaved product may be treated with a “capping agent” in order to neutralize reactive species generated in the cleavage reaction. Suitable capping agents for this purpose include amines, e.g., ethanolamine or propanolamine. In embodiments, cleavage may be accomplished by using a modified nucleotide as the cleavable site (e.g., uracil, 8oxoG, 5-mC, 5-hmC) that is removed or nicked via a corresponding DNA glycosylase, endonuclease, or combination thereof.

In embodiments, the cleavable linker as described herein (e.g., the first cleavable linker as described herein, the second cleavable linker as described herein, the third cleavable linker as described herein, and/or the fourth cleavable linker as described herein) includes two or more cleavable sites. Any suitable enzymatic, chemical, or photochemical cleavage reaction may be used to cleave the cleavable site. In embodiments, the cleavable site includes one or more deoxyuracil nucleobases (dUs). In embodiments, the cleavable site includes multiple deoxyuracil nucleobases (dUs). In embodiments, the cleavable site includes a plurality of consecutive nucleobases (dUs). In embodiments, the cleavable site is cleaved as a result of enzymatic cleaving. In embodiments, the cleaving agent is an enzyme. In embodiments, the enzyme is one or more restriction enzymes. The restriction enzyme will recognize a particular restriction site sequences in one or both strands of the cleavable site, resulting in cleavage of the cleavable site. The resulting restriction enzyme digestion may cleave one or both strands of a duplex template. The enzymatic cleavage reaction may result in removal of a part or the whole of the strand being cleaved. In embodiments, the restriction enzyme recognition sequence included in the cleavable site is selected to be a “rare-cutting” restriction enzyme recognition sequence, e.g., a restriction enzyme that cuts with low frequency in any given genome. For example, Nod is a rare cutter with an eight-base recognition site, which will occur on average about once every 65,000 base pairs in a genome (assuming an average frequency of each type of canonical base of ¼). Other rare-cutting enzymes are known in the art and commercially available, including AbsI, AscI, BbvCI, CciNI, FseI, MreI, PaIAI, RigI, SdaI, and SgsI.

In embodiments, the cleavable linker as described herein (e.g., the first cleavable linker as described herein, the second cleavable linker as described herein, the third cleavable linker as described herein, and/or the fourth cleavable linker as described herein) include one or more cleavable sites. In embodiments, the cleavable site includes one or more deoxyuracil triphosphates (dUTPs), deoxy-8-oxo-guanine triphosphates (d-8-oxoGs), methylated nucleotides, or ribonucleotides. In embodiments, the cleavable site includes one or more deoxyuracil triphosphates (dUTPs). In embodiments, the cleavable site includes one or more deoxy-8-oxo-guanine triphosphates (d-8-oxoGs). In embodiments, the cleavable site includes one or more methylated nucleotides. In embodiments, the cleavable site includes one or more ribonucleotides. The one or more cleavable sites may include a modified nucleotide, ribonucleotide, or a sequence containing a modified or unmodified nucleotide that is specifically recognized by a cleavage agent. The cleavable site(s) may be deoxyuracil triphosphate (dUTP), deoxy-8-Oxo-guanine triphosphate (d-8-oxoG), or other modified nucleotide(s), such as those described, for example, in US 2012/0238738, which is incorporated herein by reference for all purposes, and include modified ribonucleotides and deoxyribonucleotides including abasic sugar phosphates, inosine, deoxyinosine, 2,6-diamino-4-hydroxy-5-formamidopyrimidine (foramidopyrimidine-guanine, (fapy)-guanine), 8-oxoadenine, 1,N6-ethenoadenine, 3-methyladenine, 4,6-diamino-5-formamidopyrimidine, 5,6-dihydrothymine, 5,6-dihydroxyuracil, 5-formyluracil, 5-hydroxy-5-methylhydanton, 5-hydroxycytosine, 5-hydroxymethylcystosine, 5-hydroxymethyluracil, 5-hydroxyuracil, 6-hydroxy-5,6-dihydrothymine, 6-methyladenine, 7,8-dihydro-8-oxoguanine (8-oxoguanine), 7-methylguanine, aflatoxin B1-fapy-guanine, fapy-adenine, hypoxanthine, methyl-fapy-guanine, methyltartonylurea and thymine glycol. In embodiments, the cleavable site includes an abasic site, deoxyuracil triphosphate (dUTP), deoxy-8-Oxo-guanine triphosphate (d-8-oxoG), methylated nucleotide, ribonucleotide, or a sequence containing a modified or unmodified nucleotide that is specifically recognized by a cleaving agent. In embodiments, the cleavable site includes one or more ribonucleotides. In embodiments, the cleavable site includes 2 to 5 ribonucleotides. In embodiments, the cleavable site includes one ribonucleotide. In embodiments, the cleavable sites can be cleaved at or near a modified nucleotide or bond by enzymes or chemical reagents, collectively referred to here and in the claims as “cleaving agents.” Examples of cleaving agents include DNA repair enzymes, glycosylases, DNA cleaving endonucleases, or ribonucleases. For example, cleavage at dUTP may be achieved using uracil DNA glycosylase and endonuclease VIII (USER™, NEB, Ipswich, Mass.), as described in U.S. Pat. No. 7,435,572. In embodiments, when the modified nucleotide is a ribonucleotide, the cleavable site can be cleaved with an endoribonuclease. In embodiments, cleaving an extension product includes contacting the cleavable site with a cleaving agent, wherein the cleaving agent includes a reducing agent, sodium periodate, RNase, formamidopyrimidine DNA glycosylase (Fpg), endonuclease, restriction enzyme, or uracil DNA glycosylase (UDG). In embodiments, the cleaving agent is an endonuclease enzyme such as nuclease P1, AP endonuclease, T7 endonuclease, T4 endonuclease IV, Bal 31 endonuclease, Endonuclease I (endo I), Micrococcal nuclease, Endonuclease II (endo VI, exo III), nuclease BAL-31 or mung bean nuclease. In embodiments, the cleaving agent includes a restriction endonuclease, including, for example a type IIS restriction endonuclease. In embodiments, the cleaving agent is an exonuclease (e.g., RecBCD), restriction nuclease, endoribonuclease, exoribonuclease, or RNase (e.g., RNAse I, II, or III). In embodiments, the cleaving agent is a restriction enzyme. In embodiments, the cleaving agent includes a glycosylase and one or more suitable endonucleases. In embodiments, cleavage is performed under alkaline (e.g., pH greater than 8) buffer conditions at between 40° C. to 80° C.

In some embodiments, the cleaving agent includes one or more restriction endonucleases. When employing restriction endonucleases for cleavage, careful selection of the restriction endonuclease is beneficial, given the need for high efficiency cleavage and the fact that efficiency of cleavage can vary significantly according to the specific restriction endonuclease. Using a novel single molecule counting approach, Zhang et al. (see, Zhang Y et al. PLoS ONE. 2020. 15(12): e0244464, which is incorporated herein by reference in its entirety) precisely determined the cleavage efficiency of a variety of common restriction enzymes and the CRISPR-Cas9 nuclease. Zhang reported single enzyme digestion efficiencies ranging from as low as 67.12% for NdeI to as high as 99.53% for EcoRI-HF. Importantly, Zhang notes that the duration of digestion has minimal effect on the overall digestion efficiency such that the fraction of digested templates is nearly unchanged after the first 5 minutes of incubation, suggesting that a 5-minute incubation time serves as a reasonable starting point for optimization of many candidate restriction endonucleases.

In embodiments, the cleaving agent includes a single restriction endonuclease. In embodiments, the restriction endonuclease may include XbaI, EcoRI-HF, NheI, BamHI, XcmI, PflMI, BstEII, NcoI, HpaI, BsgI, AfeI, StuI, BsrGI, or a CRISPR-Cas9 nuclease (e.g., to achieve an approximate 95% cleavage or digestion rate, or the cleaving activity). In embodiments, the restriction endonuclease may include XbaI, EcoRI, BamHI, XcmI or BstEII (e.g., to achieve an approximate 98% or greater cleavage or digestion rate, or the cleaving activity). In embodiments, the restriction endonuclease may include EcoRI or XbaI (e.g., to achieve an approximate 99% or greater cleavage or digestion rate, or the cleaving activity). In some embodiments, the efficiency of cleavage may be further improved by inclusion of more than one restriction enzyme recognition site between the adapter (e.g., adapter including a platform primer binding sequence and/or sequencing primer binding sequence) and insert sequence. In some embodiments, multiple restriction endonucleases may be used in combination to precisely tune the cleavage efficiency. For example, in embodiments where >99.5% cleavage efficiency is required, a suitable dual restriction endonuclease cleavage solution may include XbaI (99.25% efficiency, as reported in Zhang) and NdeI (67.12% efficiency, as reported in Zhang), while the library constructs contain recognition sites for both XbaI and NdeI. Here, the estimated combined cleavage efficiency of the dual restriction endonuclease system is approximately 1-(1−0.9925)(1−0.6712)=99.83%.

In embodiments, cleaving includes maintaining suitable reaction conditions to permit efficient cleavage (e.g., buffer, pH, temperature conditions). In embodiments, cleaving is performed at about 20° C. to about 60° C. In embodiments, cleavage is performed at about 20° C. to about 30° C., about 30° C. to about 40° C., about 40° C. to about 50° C., or about 50° C. to about 60° C. In embodiments, cleavage is performed at about 20° C., about 25° C., about 30° C., about 35° C., about 37° C., about 40° C., about 42° C., about 45° C., about 48° C., about 50° C., about 55° C., or about 60° C. In embodiments, cleavage is performed at less than 20° C. In embodiments, cleavage is performed at greater than 60° C.

In embodiments, cleavage is performed for about 5 seconds (sec) to about 24 hours (hrs). In embodiments, cleavage is performed for about 5 sec to about 30 sec, about 30 sec to about 60 sec, about 1 minute (min) to about 5 min, about 5 min to about 15 min, about 15 min to about 30 min, about 30 min to about 60 min, about 1 hr to about 4 hrs, about 4 hrs to about 12 hrs, or about 12 hrs to about 24 hrs. In embodiments, cleavage is performed for about 5 sec, 15 sec, 30 sec, 45 sec, 1 min, 2 min, 3 min, 4 min, 5 min, 6 min, 7 min, 8 min, 9 min, 10 min, 11 min, 12 min, 13 min, 14 min, or about 15 min. In embodiments, cleavage is performed for about 20 min, 25 min, 30 min, 35 min, 40 min, 45 min, 50 min, 55 min, or about 1 hr. In embodiments, cleavage is performed for about 2 hrs, 3 hrs, 4 hrs, 5 hrs, 6 hrs, 7 hrs, 8 hrs, 9 hrs, 10 hrs, 11 hrs, or about 12 hrs. In embodiments, cleavage is performed for about 14 hrs, 16 hrs, 18 hrs, 20 hrs, 22 hrs, or about 24 hrs.

In embodiments, cleavage is performed with about 1 unit (U) to about 50 U of restriction endonuclease. The term “unit (U)” or “enzyme unit (U)” is used in accordance with its plain and ordinary meaning, and refers to the amount of the enzyme that catalyzes the conversion of one micromole of substrate per minute under the specified conditions of a given assay. In embodiments, cleavage is performed with about 1 U to about 5 U of restriction endonuclease. In embodiments, cleavage is performed with about 5 U to about 10 U of restriction endonuclease. In embodiments, cleavage is performed with about 10 U to about 15 U of restriction endonuclease. In embodiments, cleavage is performed with about 15 U to about 20 U of restriction endonuclease. In embodiments, cleavage is performed with about 20 U to about 25 U of restriction endonuclease. In embodiments, cleavage is performed with about 25 U to about 35 U of restriction endonuclease. In embodiments, cleavage is performed with about 35 U to about 50 U of restriction endonuclease. In embodiments, cleavage is performed with about 1, 2, 3,4, 5, 6, 7, 8, 9, 10, 11, 12, 13, 14, 15, 16, 17, 18, 19, 20, 21, 22, 23, 24, 25, 30, 35, 40, 45 or 50 U of restriction endonuclease. In embodiments, cleavage is performed with less than about 1 U of restriction endonuclease. In embodiments, cleavage is performed with greater than about 50 U of restriction endonuclease.

In embodiments, the method further includes amplifying a nucleic acid molecule to generate amplification products. In embodiments, amplifying includes contacting the flow cell assembly as described herein with one or more reagents for amplifying the target polynucleotide. Examples of reagents include but are not limited to polymerase, buffer, and nucleotides (e.g., an amplification reaction mixture). In certain embodiments the term “amplifying” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In embodiments, amplifying generates an amplicon. In embodiments, amplifying generates a rolony. In embodiments, an amplicon contains multiple, tandem copies of the circularized nucleic acid molecule of the corresponding sample nucleic acid. The number of copies can be varied by appropriate modification of the amplification reaction including, for example, varying the number of amplification cycles run, using polymerases of varying processivity in the amplification reaction and/or varying the length of time that the amplification reaction is run, as well as modification of other conditions known in the art to influence amplification yield. Generally, the number of copies of a nucleic acid in an amplicon is at least 100, 200, 500, 1000, 2000, 3000, 4000, 5000, 6000, 7000, 8000, 9000 and 10,000 copies, and can be varied depending on the application. As disclosed herein, one form of an amplicon is as a nucleic acid “ball” localized to the particle and/or well of the array. The number of copies of the nucleic acid can therefore provide a desired size of a nucleic acid “ball” or a sufficient number of copies for subsequent analysis of the amplicon, e.g., sequencing.

In embodiments, amplifying includes bridge polymerase chain reaction (bPCR) amplification, solid-phase rolling circle amplification (RCA), solid-phase exponential rolling circle amplification (eRCA), solid-phase recombinase polymerase amplification (RPA), solid-phase helicase dependent amplification (HDA), template walking amplification, or emulsion PCR on particles, or combinations of the methods. In embodiments, amplifying includes a bridge polymerase chain reaction amplification. In embodiments, amplifying includes a thermal bridge polymerase chain reaction (t-bPCR) amplification. In embodiments, amplifying includes a chemical bridge polymerase chain reaction (c-bPCR) amplification. Chemical bridge polymerase chain reactions include fluidically cycling a denaturant (e.g., formamide) and one or more additives (e.g., ethylene glycol) and maintaining the temperature within a narrow temperature range (e.g., +/−5° C.) or isothermally. In embodiments, c-bPCR does not include isothermal amplification, rather it requires minor (e.g., +/−5° C.) thermal oscillations. In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a much lower concentration than traditional chemical bridge polymerase chain reactions. In embodiments, amplifying includes generating a double-stranded amplification product.

It will be appreciated that any of the amplification methodologies described herein or known in the art can be utilized with universal or target-specific primers to amplify the target polynucleotide. Suitable methods for amplification include, but are not limited to, the polymerase chain reaction (PCR), strand displacement amplification (SDA), transcription mediated amplification (TMA) and nucleic acid sequence-based amplification (NASBA), for example, as described in U.S. Pat. No. 8,003,354, which is incorporated herein by reference in its entirety. The above amplification methods can be employed to amplify one or more nucleic acids of interest. Additional examples of amplification processes include, but are not limited to, bridge-PCR, recombinase polymerase amplification (RPA), loop-mediated isothermal amplification (LAMP), rolling circle amplification (RCA), strand displacement amplification (SDA), rolling circle amplification (RCA) with exponential strand displacement amplification. In embodiments, amplification includes an isothermal amplification reaction. In embodiments, amplification includes bridge amplification. In general, bridge amplification uses repeated steps of annealing of primers to templates, primer extension, and separation of extended primers from templates. Because primers are attached within the core polymer, the extension products released upon separation from an initial template is also attached within the core. The 3′ end of an amplification product is then permitted to anneal to a nearby reverse primer that is also attached within the core, forming a “bridge” structure. The reverse primer is then extended to produce a further template molecule that can form another bridge. In embodiments, forward and reverse primers hybridize to primer binding sites that are specific to a particular target nucleic acid. In embodiments, forward and reverse primers hybridize to primer binding sites that have been added to, and are common among, target polynucleotides. Adding a primer binding site to target nucleic acids can be accomplished by any suitable method, examples of which include the use of random primers having common 5′ sequences and ligating adapter nucleotides that include the primer binding site. Examples of additional clonal amplification techniques include, but are not limited to, bridge PCR, solid-phase rolling circle amplification (RCA), solid-phase exponential rolling circle amplification, solid-phase recombinase polymerase amplification (RPA), solid-phase helicase dependent amplification (HDA), template walking amplification, emulsion PCR on particles (beads), or combinations of the aforementioned methods. Optionally, during clonal amplification, additional solution-phase primers can be supplemented in the microplate for enabling or accelerating amplification. In embodiments, the amplifying includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101(43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyberbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, and Pol I DNA polymerases. SD polymerase, Bst large fragment polymerase, or a phi29 polymerase or mutant thereof.

In embodiments, the first excitation light and second excitation light are directed from a light source, where the light source includes a laser, LED (light emitting diode), a mercury or tungsten lamp, or a super-continuous diode. In embodiments, the first excitation light and second excitation light independently have a maximum excitation wavelength between 400-450 nm. In embodiments, the first excitation light and second excitation light independently have a maximum excitation wavelength between 450-500 nm. In embodiments, the first excitation light and second excitation light independently have a maximum excitation wavelength between 500-550 nm. In embodiments, the first excitation light and second excitation light independently have a maximum excitation wavelength between 550-600 nm. In embodiments, the first excitation light and second excitation light independently have a maximum excitation wavelength between 600-650 nm. In embodiments, the first excitation light and second excitation light independently have a maximum excitation wavelength between 650-700 nm. In embodiments, the first excitation light and second excitation light independently have a maximum excitation wavelength between 700-750 nm. In embodiments, the first excitation light and second excitation light independently have a maximum excitation wavelength between 750-800 nm. In embodiments, the first excitation light and second excitation light independently have a maximum excitation wavelength of 325 nm, 343 nm, 350 nm, 353 nm, 359 nm, 360 nm, 395 nm, 400 nm, 401 nm, 402 nm, 403 nm, 425 nm, 434 nm, 440 nm, 466 nm, 480 nm, 485 nm, 489 nm, 490 nm, 492 nm, 493 nm, 494 nm, 495 nm, 496 nm, 498 nm, 499 nm, 500 nm, 502 nm, 503 nm, 505 nm, 517 nm, 518 nm, 520 nm, 525 nm, 528 nm, 530 nm, 531 nm, 535 nm, 542 nm, 544 nm, 547 nm, 550 nm, 553 nm, 554 nm, 558 nm, 560 nm, 561 nm, 562 nm, 565 nm, 567 nm, 570 nm, 572 nm, 579 nm, 581 nm, 589 nm, 590 nm, 591 nm, 593 nm, 596 nm, 610 nm, 631 nm, 632 nm, 638 nm, 650 nm, 652 nm, 654 nm, 663 nm, 675 nm, 680 nm, 692 nm, 696 nm, 743 nm, 752 nm, 777 nm, or 782 nm.

In embodiments, the emission light detected after step (i) (i.e., directing a first excitation light and second excitation light at the reaction vessel) has a maximum emission wavelength between 400-450 nm. In embodiments, the emission light detected after step (i) (i.e., directing a first excitation light and second excitation light at the reaction vessel) has a maximum emission wavelength between 450-500 nm. In embodiments, the emission light detected after step (i) (i.e., directing a first excitation light and second excitation light at the reaction vessel) has a maximum emission wavelength between 500-550 nm. In embodiments, the emission light detected after step (i) (i.e., directing a first excitation light and second excitation light at the reaction vessel) has a maximum emission wavelength between 550-600 nm. In embodiments, the emission light detected after step (i) (i.e., directing a first excitation light and second excitation light at the reaction vessel) has a maximum emission wavelength between 600-650 nm. In embodiments, the emission light detected after step (i) (i.e., directing a first excitation light and second excitation light at the reaction vessel) has a maximum emission wavelength between 650-700 nm. In embodiments, the emission light detected after step (i) (i.e., directing a first excitation light and second excitation light at the reaction vessel) has a maximum emission wavelength between 700-750 nm. In embodiments, the emission light detected after step (i) (i.e., directing a first excitation light and second excitation light at the reaction vessel) has a maximum emission wavelength between 750-800 nm. In embodiments, the emission light detected after step (i) (i.e., directing a first excitation light and second excitation light at the reaction vessel) has a maximum emission wavelength between 800-850 nm. In embodiments, the emission light detected after step (i) (i.e., directing a first excitation light and second excitation light at the reaction vessel) has a maximum emission of 410 nm, 420 nm, 421 nm, 423 nm, 432 nm, 442 nm, 445 nm, 455 nm, 506 nm, 512 nm, 514 nm, 517 nm, 518 nm, 519 nm, 520 nm, 521 nm, 523 nm, 525 nm, 528 nm, 533 nm, 537 nm, 539 nm, 540 nm, 542 nm, 548 nm, 550 nm, 551 nm, 554 nm, 555 nm, 556 nm, 565 nm, 568 nm, 570 nm, 572 nm, 573 nm, 574 nm, 575 nm, 576 nm, 578 nm, 580 nm, 590 nm, 591 nm, 594 nm, 595 nm, 596 nm, 603 nm, 605 nm, 613 nm, 615 nm, 617 nm, 618 nm, 619 nm, 620 nm, 629 nm, 630 nm, 640 nm, 647 nm, 648 nm, 658 nm, 660 nm, 668 nm, 670 nm, 673 nm, 675 nm, 691 nm, 694 nm, 695 nm, 702 nm, 712 nm, 719 nm, 767 nm, 776 nm, 778 nm, 794 nm, or 804 nm.

In embodiments, the emission light detected after step (ii) (i.e., adding a first cleaving agent into the reaction vessel) has a maximum emission wavelength between 400-450 nm. In embodiments, the emission light detected after step (ii) (i.e., adding a first cleaving agent into the reaction vessel) has a maximum emission wavelength between 450-500 nm. In embodiments, the emission light detected after step (ii) (i.e., adding a first cleaving agent into the reaction vessel) has a maximum emission wavelength between 500-550 nm. In embodiments, the emission light detected after step (ii) (i.e., adding a first cleaving agent into the reaction vessel) has a maximum emission wavelength between 550-600 nm. In embodiments, the emission light detected after step (ii) (i.e., adding a first cleaving agent into the reaction vessel) has a maximum emission wavelength between 600-650 nm. In embodiments, the emission light detected after step (ii) (i.e., adding a first cleaving agent into the reaction vessel) has a maximum emission wavelength between 650-700 nm. In embodiments, the emission light detected after step (ii) (i.e., adding a first cleaving agent into the reaction vessel) has a maximum emission wavelength between 700-750 nm. In embodiments, the emission light detected after step (ii) (i.e., adding a first cleaving agent into the reaction vessel) has a maximum emission wavelength between 750-800 nm. In embodiments, the emission light detected after step (ii) (i.e., adding a first cleaving agent into the reaction vessel) has a maximum emission wavelength between 800-850 nm. In embodiments, the emission light detected after step (ii) (i.e., adding a first cleaving agent into the reaction vessel) has a maximum emission of 410 nm, 420 nm, 421 nm, 423 nm, 432 nm, 442 nm, 445 nm, 455 nm, 506 nm, 512 nm, 514 nm, 517 nm, 518 nm, 519 nm, 520 nm, 521 nm, 523 nm, 525 nm, 528 nm, 533 nm, 537 nm, 539 nm, 540 nm, 542 nm, 548 nm, 550 nm, 551 nm, 554 nm, 555 nm, 556 nm, 565 nm, 568 nm, 570 nm, 572 nm, 573 nm, 574 nm, 575 nm, 576 nm, 578 nm, 580 nm, 590 nm, 591 nm, 594 nm, 595 nm, 596 nm, 603 nm, 605 nm, 613 nm, 615 nm, 617 nm, 618 nm, 619 nm, 620 nm, 629 nm, 630 nm, 640 nm, 647 nm, 648 nm, 658 nm, 660 nm, 668 nm, 670 nm, 673 nm, 675 nm, 691 nm, 694 nm, 695 nm, 702 nm, 712 nm, 719 nm, 767 nm, 776 nm, 778 nm, 794 nm, or 804 nm.

In embodiments, the first cleavable linker and the second cleavable linker is an enzyme-cleavable linker. In embodiments, the first cleavable linker and the second cleavable linker is a photocleavable linker. In embodiments, the first cleavable linker and the second cleavable linker is an acid-cleavable linker. In embodiments, the first cleavable linker and the second cleavable linker is a base-cleavable linker. In embodiments, the first cleavable linker and the second cleavable linker is an oxidant-cleavable linker. In embodiments, the first cleavable linker and the second cleavable linker is a reductant-cleavable linker. In embodiments, the first cleavable linker and the second cleavable linker is a fluoride-cleavable linker.

In embodiments, the third cleavable linker and the fourth cleavable linker is an enzyme-cleavable linker. In embodiments, the third cleavable linker and the fourth cleavable linker is a photocleavable linker. In embodiments, the third cleavable linker and the fourth cleavable linker is an acid-cleavable linker. In embodiments, the third cleavable linker and the fourth cleavable linker is a base-cleavable linker. In embodiments, the third cleavable linker and the fourth cleavable linker is an oxidant-cleavable linker. In embodiments, the third cleavable linker and the fourth cleavable linker is a reductant-cleavable linker. In embodiments, the third cleavable linker and the fourth cleavable linker is a fluoride-cleavable linker.

In embodiments, the first nucleotide includes a reversible terminator. In embodiments, the second nucleotide includes a reversible terminator. In embodiments, the third nucleotide includes a reversible terminator. In embodiments, the fourth nucleotide includes a reversible terminator.

In embodiments, the first nucleotide, the second nucleotide, the third nucleotide, and the fourth nucleotide each independently include the formula:

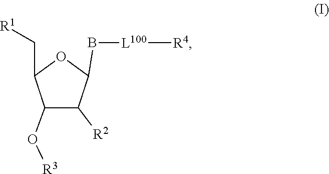

wherein B, R¹, R², R³, R⁴, and L¹⁰⁰ are described herein.

In embodiments, the first nucleotide, the second nucleotide, the third nucleotide, and the fourth nucleotide each independently include the formula:

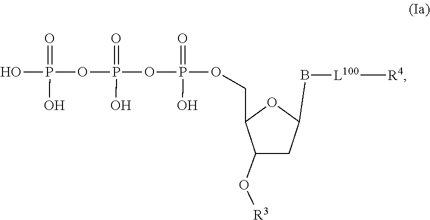

wherein B, R³, R⁴, and L¹⁰⁰ are described herein.

In embodiments, the first nucleotide, the second nucleotide, the third nucleotide, and the fourth nucleotide are selected from the following group:

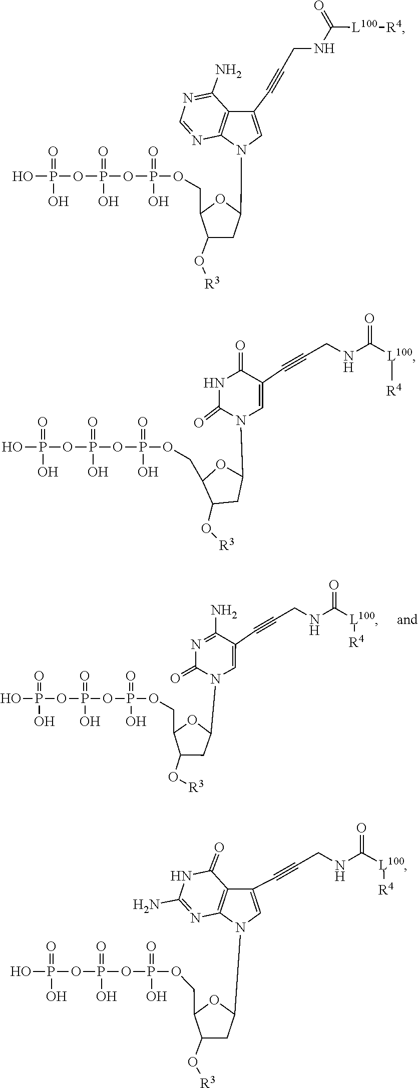

wherein R³, R⁴, and L¹⁰⁰ are described herein.

In embodiments, L¹⁰⁰ is a divalent linker including

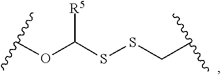

wherein R⁵ is described herein. In embodiments, L¹⁰⁰ is a divalent linker including

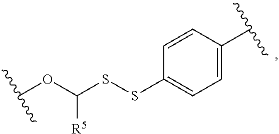

wherein R⁵ is described herein.

In embodiments, L¹⁰⁰ is a divalent linker including

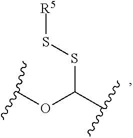

wherein R⁵ is described herein. In embodiments, L¹⁰⁰ is a divalent linker including

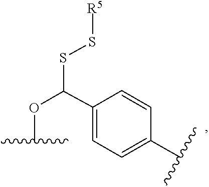

wherein R⁵ is described herein. In embodiments, L¹⁰⁰ is a divalent linker including

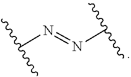 [SMILES: */N=N/*]

In embodiments, L¹⁰⁰ is a divalent linker including

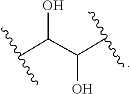 [SMILES: *C(O)C(*)O]

In embodiments, L¹⁰⁰ is a divalent linker including

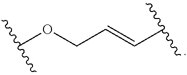 [SMILES: */C=C/CO*]

In embodiments, L¹⁰⁰ is a divalent linker including

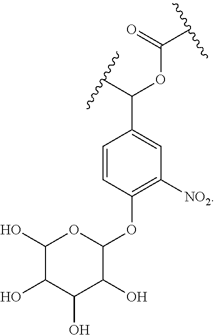 [SMILES: *C(=O)OC(*)c1ccc(OC2OC(O)C(O)C(O)C2O)c([N+](=O)[O-])c1]

In embodiments, L¹⁰⁰ is an enzymatically cleavable linker, wherein L¹⁰⁰ includes a Cathepsin B substrate moiety (e.g., a moiety including

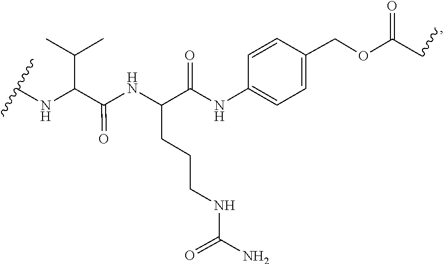

valine-citrulline dipeptide, alanine-citrulline dipeptide, z-Arginine-Arginine-para-nitroanalide, or z-Arginine-Arginine-amino-4-methylcoumarin as described in Zheng, Su. Acta Pharmaceutica Sinica B. 2021 December; 11(12): 3889-3907 and Yoon, M. et al. Biochemistry. 2023 Aug. 1; 62(15): 2289-2300), β-glucuronidase substrate moiety (e.g., a moiety including

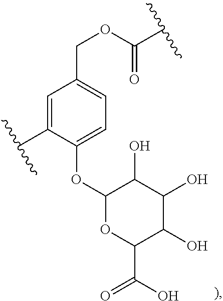 [SMILES: *C(=O)OCc1ccc(OC2OC(C(=O)O)C(O)C(O)C2O)c(*)c1]

β-galactosidase substrate moiety (e.g., a moiety including

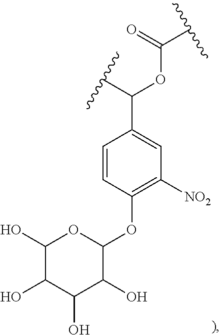 [SMILES: *C(=O)OC(*)c1ccc(OC2OC(O)C(O)C(O)C2O)c([N+](=O)[O-])c1]

a sulfatase substrate moiety (e.g., a moiety including

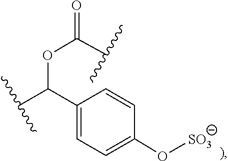

or derivatives thereof. In embodiments, L¹⁰⁰ is a divalent enzymatically cleavable linker including a β-galactosidase substrate moiety. In embodiments, L¹⁰⁰ is a divalent linker that is cleavable in the presence of a reducing agent, wherein L¹⁰⁰ includes a disulfide moiety or an azo moiety. In embodiments, L¹⁰⁰ is a divalent linker that is cleavable in the presence of an oxidative agent, wherein L¹⁰⁰ includes vicinal (i.e., 1,2-diol) diol moiety or selenium containing moiety. In embodiments, L¹⁰⁰ is a divalent linker that is a photocleavable linker, wherein L¹⁰⁰ includes 2-nitrobenzyl moiety (i.e., ortho-nitrobenzyl), phenacyl ester moiety, 8-quinolinyl benzenesulfonate moiety, or dicoumarin moiety, or bis-arylhydrazone moiety. In embodiments, L¹⁰⁰ is a divalent linker that is cleavable in the presence of a base, wherein L¹⁰⁰ includes cyanoethyl moiety or thioester moiety. In embodiments, L¹⁰⁰ is a divalent linker that is cleavable in the presence of an acid, wherein L¹⁰⁰ includes an acetal moiety, cyclic acetal moiety, dialkylketal moiety, silyl ether moiety, or hydrazone moiety (see, e.g., Leriche, G. et al. Bioorg Med Chem. 2012 Jan. 15; 20(2):571-82). In embodiments, L¹⁰⁰ is a divalent linker including a polynucleotide sequence. In embodiments, L¹⁰⁰ is a divalent linker including a polypeptide sequence (e.g., a linker including a 15-residue (Gly₄Ser)₃ peptide).

In embodiments, the first cleavable linker and the second cleavable linker include a disulfide moiety. In embodiments, the first cleavable linker and the second cleavable linker include a dialkylketal moiety. In embodiments, the first cleavable linker and the second cleavable linker include an allyl moiety. In embodiments, the first cleavable linker and the second cleavable linker include an azide moiety. In embodiments, the first cleavable linker and the second cleavable linker include a hydrazine moiety. In embodiments, the first cleavable linker and the second cleavable linker include a cyanoethyl moiety. In embodiments, the first cleavable linker and the second cleavable linker include a nitrobenzyl moiety.

In embodiments, prior to adding an extension solution comprising four nucleotides, the method further includes forming a circular oligonucleotide via ligation. In embodiments, the ligation includes enzymatic ligation. In embodiments, the two ends of an extended oligonucleotide primer are ligated directly together. In embodiments, the two ends of the extended oligonucleotide primer are ligated together with the aid of a bridging oligonucleotide (sometimes referred to as a splint oligonucleotide) that is complementary with the two ends of the extended oligonucleotide primer. In embodiments, ligating includes enzymatic ligation including a ligation enzyme (e.g., Circligase enzyme, Taq DNA Ligase, HiFi Taq DNA Ligase, T4 ligase, PBCV-1 DNA Ligase (also known as SplintR™ ligase) or Ampligase DNA Ligase). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR ligase) or a Taq DNA Ligase. In embodiments, ligating includes chemical ligation (e.g., enzyme-free, click-mediated ligation). In embodiments, the oligonucleotide primer includes a first bioconjugate reactive moiety capable of bonding upon contact with a second (complementary) bioconjugate reactive moiety.

In embodiments, the circular oligonucleotide is about 100 to about 1000 nucleotides in length. In embodiments, the circular oligonucleotide is about 100 nucleotides, about 110 nucleotides, about 120 nucleotides, about 130 nucleotides, about 140 nucleotides, about 150 nucleotides, about 160 nucleotides, about 170 nucleotides, about 180 nucleotides, about 190 nucleotides, or about 200 nucleotides. In embodiments, the circular oligonucleotide is about 100 nucleotides, about 150 nucleotides, about 200 nucleotides, about 250 nucleotides, about 300 nucleotides, about 350 nucleotides, about 400 nucleotides, about 450 nucleotides, about 500 nucleotides, about 550 nucleotides, about 600 nucleotides, about 650 nucleotides, about 700 nucleotides, about 750 nucleotides, about 800 nucleotides, about 850 nucleotides, about 900 nucleotides, about 950 nucleotides, or about 1000 nucleotides. Circular oligonucleotides may be conveniently isolated by a conventional purification column, digestion of non-circular DNA by one or more appropriate exonucleases, or both.

In embodiments, the method includes amplifying the circular oligonucleotide. In embodiments, the method includes amplifying the circular oligonucleotide in the cell or tissue. In embodiments, the method further includes amplifying the circular oligonucleotide by extending an amplification primer hybridized to the circular oligonucleotide with a strand-displacing polymerase, wherein the amplification primer extension generates an extension product including multiple complements of the circular oligonucleotide. In embodiments, the method further includes sequencing the extension product.

In embodiments, the method includes removing the circular oligonucleotide (e.g., for further processing, such as enrichment and/or amplification, and detection). In embodiments, removing the oligonucleotide includes incubation in a denaturant, for example, wherein the denaturant is a buffered solution including about 0% to about 50% dimethyl sulfoxide (DMSO); about 0% to about 50% ethylene glycol; about 0% to about 20% formamide; or about 0 to about 3M betaine, or a mixture thereof. Incubation in a denaturant should only remove the circular oligonucleotide and not remove the bound probes from the protein(s). Optimization of denaturant conditions may be performed to identify conditions suitable for selective denaturation. In embodiments, the reaction conditions are modified to denaturing conditions by i) increasing the temperature, ii) contacting the oligonucleotide with a chemical denaturant, or iii) a combination thereof.

In embodiments, amplifying includes hybridizing an amplification primer to the circular oligonucleotide and extending the primer with a strand-displacing polymerase to generate an amplification product including multiple complements of the circular oligonucleotide. In embodiments, the method includes amplifying the circular oligonucleotide by extending an amplification primer hybridized to the circular oligonucleotide with a strand-displacing polymerase, wherein the amplification primer extension generates an extension product including multiple complements of the circular oligonucleotide. In embodiments, the amplification primer extension generates an extension product including one or more complements of the circular oligonucleotide. In embodiments, the amplification primer extension generates an extension product including 2, 3, 4, 5, 6, 7, 8, 9, 10, 11, 12, 13, 14, 15, 16, 17, 18, 19, 20, or more complements of the circular oligonucleotide. In embodiments, the method further includes sequencing the extension product.

In embodiments, the method includes amplifying the circular polynucleotide of the cell in situ. In embodiments, amplifying the circular polynucleotide generates an amplification product. In embodiments, the amplification product includes three or more copies of the circular polynucleotide. In embodiments, the amplification product includes at least three or more copies of the circular polynucleotide. In embodiments, the amplification product includes at least five or more copies of the circular polynucleotide. In embodiments, the amplification product includes at 5 to 10 copies of the circular polynucleotide. In embodiments, the amplification product includes 10 to 20 copies of the circular polynucleotide. In embodiments, the amplification product includes 20 to 50 copies of the circular polynucleotide.

In embodiments, amplifying the circular polynucleotide includes incubating the circular polynucleotide with the strand-displacing polymerase (a) for about 1 minute to about 2 hours, and/or (b) at a temperature of about 20° C. to about 50° C. In embodiments, amplifying the circular polynucleotide includes incubating the circular polynucleotide with the strand-displacing polymerase for about 1 minute to about 2 hours. In embodiments, amplifying the circular polynucleotide includes incubating the circular polynucleotide with the strand-displacing polymerase for about 5, about 10, about 20, about 30, about 40, about 45, about 50, about 55, or about 60 minutes.

In embodiments, amplifying the circular polynucleotide includes incubating the circular polynucleotide with the strand-displacing polymerase for about 1 hour to about 12 hours. In embodiments, amplifying includes incubation with the strand-displacing polymerase for about 60 seconds to about 60 minutes. In embodiments, amplifying includes incubation with the strand-displacing polymerase for about 10 minutes to about 60 minutes. In embodiments, amplifying includes incubation with the strand-displacing polymerase for about 10 minutes to about 30 minutes. In embodiments, amplifying the circular polynucleotide includes incubating the circular polynucleotide with the strand-displacing polymerase for about 1, about 2, about 3, about 4, about 5, about 6, about 7, about 8, about 9, about 10, about 11, or about 12 hours. In embodiments, amplifying the circular polynucleotide includes incubating the circular polynucleotide with the strand-displacing polymerase for more than 12 hours.

In embodiments, amplifying the circular polynucleotide includes incubating the circular polynucleotide with the strand-displacing polymerase at a temperature of about 20° C. to about 50° C. In embodiments, incubation with the strand-displacing polymerase is at a temperature of about 20° C., about 25° C., about 30° C., about 35° C., about 40° C., about 45° C., or about 50° C. In embodiments, incubation with the strand-displacing polymerase is at a temperature of about 35° C. to 42° C. In embodiments, incubation with the strand-displacing polymerase is at a temperature of about 35° C., about 36° C., about 37° C., about 38° C., about 39° C., about 40° C., about 41° C., or about 42° C. In embodiments, the strand-displacing polymerase is a phi29 polymerase, a SD polymerase, a Bst large fragment polymerase, phi29 mutant polymerase, a Thermus aquaticus polymerase, or a thermostable phi29 mutant polymerase.

In embodiments, the amplifying includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101(43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyperbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, and Pol I DNA polymerases. SD polymerase, Bst large fragment polymerase, or a phi29 polymerase or mutant thereof. In embodiments, the strand-displacing enzyme is an SD polymerase, Bst large fragment polymerase, or a phi29 polymerase or mutant thereof. In embodiments, the strand-displacing polymerase is Bst DNA Polymerase Large Fragment, Thermus aquaticus (Taq) polymerase, or a mutant thereof.

In embodiments, the amplification method includes a standard dNTP mixture including dATP, dCTP, dGTP and dTTP (for DNA) or dATP, dCTP, dGTP and dUTP (for RNA). In embodiments, the amplification method includes a mixture of standard dNTPs and modified nucleotides that contain functional moieties (e.g., bioconjugate reactive groups) that serve as attachment points to the cell or the matrix in which the cell is embedded (e.g., a hydrogel). In embodiments, the amplification method includes a mixture of standard dNTPs and modified nucleotides that contain functional moieties (e.g., bioconjugate reactive groups) that participate in the formation of a bioconjugate linker. The modified nucleotides may react and link the amplification product to the surrounding cell scaffold. For example, amplifying may include an extension reaction wherein the polymerase incorporates a modified nucleotide into the amplification product, wherein the modified nucleotide includes a bioconjugate reactive moiety (e.g., an alkynyl moiety) attached to the nucleobase. The bioconjugate reactive moiety of the modified nucleotide participates in the formation of a bioconjugate linker by reacting with a complementary bioconjugate reactive moiety present in the cell (e.g., a crosslinking agent, such as NHS-PEG-azide, or an amine moiety) thereby attaching the amplification product to the internal scaffold of the cell. In embodiments, the functional moiety can be covalently cross-linked, copolymerize with or otherwise non-covalently bound to the matrix. In embodiments, the functional moiety can react with a cross-linker. In embodiments, the functional moiety can be part of a ligand-ligand binding pair. Suitable exemplary functional moieties include an amine, acrydite, alkyne, biotin, azide, and thiol. In embodiments of crosslinking, the functional moiety is cross-linked to modified dNTP or dUTP or both. In embodiments, suitable exemplary cross-linker reactive groups include imidoester (DMP), succinimide ester (NHS), maleimide (Sulfo-SMCC), carbodiimide (DCC, EDC) and phenyl azide. Cross-linkers within the scope of the present disclosure may include a spacer moiety. In embodiments, such spacer moieties may be functionalized. In embodiments, such spacer moieties may be chemically stable. In embodiments, such spacer moieties may be of sufficient length to allow amplification of the nucleic acid bound to the matrix. In embodiments, suitable exemplary spacer moieties include polyethylene glycol, carbon spacers, photo-cleavable spacers and other spacers known to those of skill in the art and the like. In embodiments, amplification reactions include standard dNTPs and a modified nucleotide (e.g., amino-allyl dUTP, 5-TCO-PEG4-dUTP, C8-Alkyne-dUTP, 5-Azidomethyl-dUTP, 5-Vinyl-dUTP, or 5-Ethynyl dLTTP). For example, during amplification a mixture of standard dNTPs and aminoallyl deoxyuridine 5′-triphosphate (dUTP) nucleotides may be incorporated into the amplicon and subsequently cross-linked to the cell protein matrix by using a cross-linking reagent (e.g., an amine-reactive crosslinking agent with PEG spacers, such as (PEGylated bis(sulfosuccinimidyl)suberate) (BS(PEG)9)).

In certain embodiments the term “amplifying” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In embodiments, amplifying generates an amplicon. In embodiments, an amplicon contains multiple, tandem copies of the circularized nucleic acid molecule of the corresponding sample nucleic acid. The number of copies can be varied by appropriate modification of the amplification reaction including, for example, varying the number of amplification cycles run, using polymerases of varying processivity in the amplification reaction and/or varying the length of time that the amplification reaction is run, as well as modification of other conditions known in the art to influence amplification yield. Generally, the number of copies of a nucleic acid in an amplicon is at least 100, 200, 500, 1000, 2000, 3000, 4000, 5000, 6000, 7000, 8000, 9000 and 10,000 copies, and can be varied depending on the application. As disclosed herein, one form of an amplicon is as a nucleic acid “ball” or “cluster” localized to the particle and/or well of the array. The number of copies of the nucleic acid can therefore provide a desired size of a nucleic acid “ball” or a sufficient number of copies for subsequent analysis of the amplicon, e.g., sequencing.

In embodiments of the methods provided herein, the amplicon clusters have a mean or median separation from one another of about 0.5-5 μm. In embodiments, the mean or median separation is about 0.1-10 microns, 0.25-5 microns, 0.5-2 microns, 1 micron, or a number or a range between any two of these values. In embodiments, the mean or median separation is about or at least about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4., 4.5, 4.6, 4.7, 4.8, 4.9, 5.0 μm or a number or a range between any two of these values. The mean or median separation may be measured center-to-center (i.e., the center of one amplicon cluster to the center of a second amplicon cluster). In embodiments of the methods provided herein, the amplicon clusters have a mean or median separation (measured center-to-center) from one another of about 0.5-5 μm. The mean or median separation may be measured edge-to-edge (i.e., the edge of one amplicon cluster to the edge of a second amplicon cluster). In embodiments of the methods provided herein, the amplicon clusters have a mean or median separation (measured edge-to-edge) from one another of about 0.2-5 μm.

In embodiments, the method further includes detecting the amplification products. In embodiments, the amplification products include the template polynucleotide described herein. In embodiments, detecting the amplification products includes detecting the label (e.g., the nucleic acid sequence or template polynucleotide described herein). In embodiments, detecting the amplification products includes detecting the oligonucleotide label (e.g., template polynucleotide described herein). In embodiments, detecting includes sequencing. In embodiments, sequencing includes extending a sequencing primer annealed to the target polynucleotide to incorporate a nucleotide containing a detectable label that indicates the identity of a nucleotide in the target polynucleotide, detecting the detectable label, and optionally repeating the extending and detecting of steps. In embodiments, the methods include sequencing one or more bases of a target nucleic acid by extending a sequencing primer hybridized to a target nucleic acid (e.g., an amplification product of a target nucleic acid). In embodiments, the sequencing includes sequencing-by-synthesis, sequencing by ligation, sequencing-by-hybridization, or pyrosequencing, and generates a sequencing read. In embodiments, generating a sequencing read includes executing a plurality of sequencing cycles, each cycle including extending the sequencing primer by incorporating a nucleotide or nucleotide analogue using a polymerase and detecting a characteristic signature indicating that the nucleotide or nucleotide analogue has been incorporated.

In embodiments, sequencing includes a plurality of sequencing cycles. In embodiments, sequencing includes 20 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue.

In embodiments, the method includes sequencing the first and/or the second strand of a amplification product by extending a sequencing primer hybridized thereto. A variety of sequencing methodologies can be used such as sequencing-by-synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, that are present at features of an array are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by an SBS process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. No. 10,738,072. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Non-limiting examples of suitable labels are described in U.S. Pat. Nos. 8,178,360, 5,188,934 (4,7-dichlorofluorscein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthene dyes): U.S. Pat. No. 5,688,648 (energy transfer dyes); and the like.

Sequencing includes, for example, detecting a sequence of signals. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the nucleotides are labeled with at least two unique fluorescent dyes. In embodiments, the readout is accomplished by epifluorescence imaging. A variety of sequencing chemistries are available, non-limiting examples of which are described herein.

Use of the sequencing method outlined above is a non-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

In embodiments, detecting includes detecting 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 transcripts per μm². In embodiments, detecting includes detecting 4, 5, 6, 7, 8, 9, 10, 11, 12, 13, 14, 15, 16, 17, 18, 19, 2, 21, 22, 23, 24, 25, 26, 27, 28, 29, or 30 transcripts per μm². In embodiments, detecting includes detecting 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, or 3 transcripts per μm³. In embodiments, detecting includes detecting 4, 5, 6, 7, 8, 9, 10, 11, 12, 13, 14, 15, 16, 17, 18, 19, 2, 21, 22, 23, 24, 25, 26, 27, 28, 29, or 30 transcripts per μm³.

In embodiments, the method further includes obtaining an image of a cell or tissue. In embodiments, the imaging includes phase-contrast microscopy, bright-field microscopy, Nomarski differential-interference-contrast microscopy, dark field microscopy, electron microscopy, or cryo-electron microscopy. In embodiments, the light transmittance of the sample is measured. For example, light transmittance may be measured with a visible near-infrared optical fiber spectrometer, wherein a circular spot of light (e.g., diameter, 5 mm) is irradiated on the central part a sample and the transmitted light is collected using an optical sensor.

In embodiments, the method further includes an imaging modality including immunofluorescence (IF), or immunohistochemistry modality (e.g., immunostaining). In embodiments, the method further includes an imaging modality including fluorescent hematoxylin and eosin (H&E) modality. For example, the method includes contacting the sample with a stain (e.g., Acridine orange, Auramine O, Calcofluor white, DAPI, Ethidium bromide, Hoechst 33258, Propidium iodide, Rhodamine B, SYBR Green, Texas Red, Thioflavin T, TOTO®-3, Uvitex 2B, YOYO®-1, 7-Aminoactinomycin D (7-AAD), TO-PRO®, TOPRO-3®, or eosin). In embodiments, the method includes ER staining (e.g., contacting the tissue section with a cell-permeable dye which localizes to the endoplasmic reticula), Golgi staining (e.g., contacting the tissue section with a cell-permeable dye which localizes to the Golgi), F-actin staining (e.g., contacting the tissue section with a phalloidin-conjugated dye that binds to actin filaments), lysosomal staining (e.g., contacting the tissue section with a cell-permeable dye that accumulates in the lysosome via the lysosome pH gradient), mitochondrial staining (e.g., contacting the tissue section with a cell-permeable dye which localizes to the mitochondria), nucleolar staining, or plasma membrane staining. For example, the method includes live cell imaging (e.g., obtaining images of the tissue section) prior to or during fixing, immobilizing, and permeabilizing the tissue section. Immunohistochemistry (IHC) is a powerful technique that exploits the specific binding between an antibody and antigen to detect and localize specific antigens in cells and tissue, commonly detected and examined with the light microscope. Known IHC modalities may be used, such as the protocols described in Magaki, S., Hojat, S. A., Wei, B., So, A., & Yong, W. H. (2019). Methods in molecular biology(Clifton, N.J.), 1897, 289-298, which is incorporated herein by reference. In embodiments, the additional imaging modality includes bright field microscopy, phase contrast microscopy, Nomarski differential-interference-contrast microscopy, or dark field microscopy. In embodiments, the method further includes determining the cell morphology of the tissue section (e.g., the cell boundary or cell shape) using known methods in the art. For example, to determining the cell boundary includes comparing the pixel values of an image to a single intensity threshold, which may be determined quickly using histogram-based approaches as described in Carpenter, A. et al Genome Biology 7, R100 (2006) and Arce, S., Sci Rep 3, 2266 (2013)). By “microscopic analysis” is meant the analysis of a specimen using techniques that provide for the visualization of aspects of a specimen that cannot be seen with the unaided eye, i.e., that are not within the resolution range of the normal human eye. Such techniques may include, without limitation, optical microscopy, e.g., bright field, oblique illumination, dark field, phase contrast, differential interference contrast, interference reflection, epifluorescence, confocal microscopy, CLARITY-optimized light sheet microscopy (COLM), light field microscopy, tissue expansion microscopy, etc., laser microscopy, such as, two photon microscopy, electron microscopy, and scanning probe microscopy. By “preparing a biological specimen for microscopic analysis” is generally meant rendering the specimen suitable for microscopic analysis at an unlimited depth within the specimen. In embodiments, the immobilized tissue section is imaged using “optical sectioning” techniques, such as laser scanning confocal microscopes, laser scanning 2-Photon microscopy, parallelized confocal (i.e. spinning disk), computational image deconvolution methods, and light sheet approaches. Optical sectioning microscopy methods provide information about single planes of a volume by minimizing contributions from other parts of the volume and do so without physical sectioning. The resulting “stack” of such optically sectioned images, represents a full reconstruction of the 3-dimensional features of a tissue volume. A typical confocal microscope includes a 10×/0.5 objective (dry; working distance, 2.0 mm) and/or a 20×/0.8 objective (dry; working distance, 0.55 mm), with a s z-step interval of 1 to 5 μm. A typical light sheet fluorescence microscope includes an sCMOS camera, a 2x/0.5 objective lens, and zoom microscope body (magnification range of ×0.63 to ×6.3). For entire scanning of whole samples, the z-step interval is 5 or 10 μm, and for image acquisition in the regions of interest, an interval in the range of 2 to 5 μm may be used.

In embodiments, the imaging modality is capable of imaging an imaging area of about 1 cm² to about 10 cm², 1 cm² to about 5 cm², 5 cm² to about 10 cm², 10 cm² to about 30 cm², 30 cm² to about 60 cm², 60 cm² to about 90 cm², 90 cm² to about 120 cm², or more.

In embodiments, the collection of information (e.g., sequencing information and cell morphology) is referred to as a signature. The term “signature” may encompass any gene or genes, protein or proteins, or epigenetic element(s) whose expression profile or whose occurrence is associated with a specific cell type, subtype, or cell state of a specific cell type or subtype within a population of cells. It is to be understood that also when referring to proteins (e.g., differentially expressed proteins), such may fall within the definition of “gene” signature. Levels of expression or activity or prevalence may be compared between different cells in order to characterize or identify for instance signatures specific for cell (sub)populations. Increased or decreased expression or activity of signature genes may be compared between different cells in order to characterize or identify for instance specific cell (sub)populations.

In an aspect is provided a method of sequencing a template polynucleotide. In embodiments, the method includes (a) contacting a first primer hybridized to the template polynucleotide with four nucleotides and incorporating with a polymerase one of the four nucleotides into the first primer, wherein two of the four nucleotides comprise a first fluorophore moiety attached to each nucleotide via a first cleavable linker and two of the four nucleotides comprise a second fluorophore moiety attached to each nucleotide via a second cleavable linker, wherein the first fluorophore moiety generates a first emission light and the second fluorophore moiety generates a second emission light; (b) determining the identity of the incorporated nucleotide by: (i) detecting the first emission light; cleaving the first cleavable linker; and detecting the absence of the first emission light; (ii) detecting the first emission light; cleaving the first cleavable linker; and detecting the first emission light again; (iii) detecting the second emission light; cleaving the first cleavable linker; and detecting the absence of the first emission light; or (iv) detecting the second emission light; cleaving the first cleavable linker; and detecting the second emission light; (c) repeating steps (a) and (b), thereby sequencing a template polynucleotide.

In an aspect is provided a method of sequencing a template polynucleotide. In embodiments, the method described herein includes (a) contacting a first primer hybridized to the template polynucleotide with four nucleotides and incorporating with a polymerase one of the nucleotides attached to a fluorophore moiety via a first cleavable linker and two of the four nucleotides includes a second fluorophore moiety attached to each nucleotide via a second cleavable linker, wherein the first fluorophore moiety generates a first emission light and the second fluorophore moiety generates a second emission light; (b) determining the identity of the incorporated nucleotide by: (i) detecting the first emission light; cleaving the first cleavable linker; and detecting the absence of the first emission light; (ii) detecting the first emission light; cleaving the first cleavable linker; and detecting the first emission light again; (iii) detecting the second emission light; cleaving the first cleavable linker; and detecting the absence of the first emission light; or (iv) detecting the second emission light; cleaving the first cleavable linker; and detecting the second emission light; (c) repeating steps (a) and (b), thereby sequencing a template polynucleotide.

In embodiments, the method described herein includes (a) contacting a first primer hybridized to the template polynucleotide with four nucleotides and incorporating with a polymerase nucleotides attached to a fluorophore moiety via a first cleavable linker and two of the four nucleotides includes a second fluorophore moiety attached to each nucleotide via a second cleavable linker, wherein the first fluorophore moiety generates a first emission light and the second fluorophore moiety generates a second emission light; (b) determining the identity of the incorporated nucleotide by detecting the first emission light, cleaving the first cleavable linker, and detecting the absence of the first emission light; (c) repeating steps (a) and (b), thereby sequencing a template polynucleotide.

In embodiments, the method described herein includes (a) contacting a first primer hybridized to the template polynucleotide with four nucleotides and incorporating with a polymerase nucleotides attached to a fluorophore moiety via a first cleavable linker and two of the four nucleotides includes a second fluorophore moiety attached to each nucleotide via a second cleavable linker, wherein the first fluorophore moiety generates a first emission light and the second fluorophore moiety generates a second emission light; (b) determining the identity of the incorporated nucleotide by detecting the first emission light, cleaving the first cleavable linker, and detecting the first emission light again; (c) repeating steps (a) and (b), thereby sequencing a template polynucleotide.

In embodiments, the method described herein includes (a) contacting a first primer hybridized to the template polynucleotide with four nucleotides and incorporating with a polymerase nucleotides attached to a fluorophore moiety via a first cleavable linker and two of the four nucleotides includes a second fluorophore moiety attached to each nucleotide via a second cleavable linker, wherein the first fluorophore moiety generates a first emission light and the second fluorophore moiety generates a second emission light; (b) determining the identity of the incorporated nucleotide by detecting the second emission light, cleaving the first cleavable linker, and detecting the second emission light; (c) repeating steps (a) and (b), thereby sequencing a template polynucleotide.

In an aspect is a provided a method of extending a primer in or on a cell or tissue. In embodiments, the method described herein includes (a) adding an extension solution comprising four nucleotides to the cell or tissue comprising a polymerase and the primer, wherein the primer is hybridized to a target polynucleotide, and incorporating one of four nucleotides into the primer, wherein the extension solution includes a first nucleotide including a first fluorophore moiety attached to the nucleotide via a first cleavable linker; a second nucleotide including a second fluorophore moiety attached to the nucleotide via a second cleavable linker; a third nucleotide including a third fluorophore moiety attached to the nucleotide via a third cleavable linker; a fourth nucleotide including a fourth fluorophore moiety attached to the nucleotide via a fourth cleavable linker, wherein the first and the second cleavable linkers are cleavable under identical conditions and the third and the fourth cleavable linkers are cleavable under different identical conditions and (b) exciting a fluorophore, wherein exciting includes (i) directing a first excitation light and second excitation light at the cell or tissue; (ii) adding a first cleaving agent to the cell or tissue; followed by (iii) adding a second cleaving agent into the cell or tissue. In embodiments, the method further includes detecting the incorporated nucleotide, wherein detecting includes detecting an emission light after step (i) (i.e., directing a first excitation light and second excitation light at the cell or tissue). In embodiments, the method further includes detecting the incorporated nucleotide, wherein detecting comprises detecting a first emission light after step (i) (i.e., directing a first excitation light and second excitation light at the cell or tissue) and detecting a second emission light after step (ii) (i.e., adding a first cleaving agent to the cell or tissue). In embodiments, the method further includes determining the identity of the incorporated nucleotide based on the detection of the emission light. In embodiments, the template polynucleotide is in a cell. In embodiments, the template polynucleotide is on a cell. In embodiments, the template polynucleotide is in a tissue.

In an aspect is provided a method of sequencing a template polynucleotide in or on a cell or tissue. In embodiments, the method includes (a) contacting a first primer hybridized to the template polynucleotide with four nucleotides and incorporating with a polymerase one of the four nucleotides into the first primer, wherein two of the four nucleotides comprise a first fluorophore moiety attached to each nucleotide via a first cleavable linker and two of the four nucleotides comprise a second fluorophore moiety attached to each nucleotide via a second cleavable linker, wherein the first fluorophore moiety generates a first emission light and the second fluorophore moiety generates a second emission light; (b) determining the identity of the incorporated nucleotide by: (i) detecting the first emission light; cleaving the first cleavable linker; and detecting the absence of the first emission light; (ii) detecting the first emission light; cleaving the first cleavable linker; and detecting the first emission light again; (iii) detecting the second emission light; cleaving the first cleavable linker; and detecting the absence of the first emission light; or (iv) detecting the second emission light; cleaving the first cleavable linker; and detecting the second emission light; (c) repeating steps (a) and (b), thereby sequencing a template polynucleotide. In embodiments, the template polynucleotide is in a cell. In embodiments, the template polynucleotide is on a cell. In embodiments, the template polynucleotide is in a tissue.

In some embodiments, samples (e.g., a sample including one or more cells or tissues, as described herein) may be pretreated. Pretreatments for increasing the availability of protein targets for interaction with specific detection reagents in situ (e.g., “antigen retrieval”) are known in the art, as exemplified by Shi et al. 1997, J. Histochem Cytochem, 45(3):327. In some embodiments, antigen retrieval may be achieved using protease-induced epitope retrieval (PIER), and may employ enzymes such as proteinase K, pepsin, trypsin, protease, and any subtypes thereof, in an appropriate buffer to restore the epitope for antibody binding. In some embodiments, antigen retrieval may be achieved using heat-induced epitope retrieval (HIER) and may employ heat to reverse some cross-links and allow the restoration of epitopes. In some embodiments, citrate buffers, Tris, and EDTA base may be employed as exemplary heat-induced reagents in appropriately pH stabilized manner (e.g., 10 mM sodium citrate, 6.0 pH; 1 mM EDTA, pH 8.0; 10 mM Tris base, 1 mM EDTA solution, 0.05% Tween 20, pH 9.0). Detergents (e.g., Tween 20) may be added to the HIER buffer to increase the epitope retrieval. In certain embodiments, many proprietary formulations are available for the PIER or HIER mediate antigen retrieval.

In some embodiments, the sample may be treated with a blocking solution, prior to introduction of the nucleotides as described herein to the sample, in order to reduce the likelihood of any unspecific binding. Typically, depending on the tissue type and the method of antigen detection, endogenous biotin or enzymes may need to be blocked or quenched, respectively, prior to antibody staining. In some embodiments, samples are incubated with a “blocking buffer” that blocks reactive sites to which probes may otherwise bind. In embodiments, the blocking buffer may include normal serum (i.e., goat serum). In embodiments, the blocking buffer may include non-fat dry milk. In embodiments, the blocking buffer may include FBS (fetal bovine serum). In embodiments, the blocking buffer may include BSA (bovine serum albumin). In embodiments, the blocking buffer may include gelatin. In embodiments, any number of commercial blocking buffers, each with proprietary formulations, may be used. There are many commercial blocking buffers available that are known in the art. In some embodiments, the sample may incubated with the blocking buffer for about 5 minutes to about 1 hour, about 5 minutes to about 40 minutes, about 5 minutes to about 30 minutes, about 5 minutes to about 20 minutes, or about 5 minutes to about 10 minutes. In some embodiments, the sample may incubated with the blocking buffer at room temperature. In some embodiments, the sample may incubated with the blocking buffer at a temperature of about 4° C. to about 35° C., about 4° C. to about 25° C., about 4° C. to about 20° C., about 4° C. to about 10° C., about 10° C. to about 25° C., about 10° C. to about 20° C., about 10° C. to about 15° C., about 35° C. to about 50° C., about 35° C. to about 45° C., about 35° C. to about 40° C., about 40° C. to about 50° C., about 40° C. to about 45° C., or about 45° C. to about 50° C.

Methods to minimize background staining in situ are known in the art. Such methods include, but are not limited to, experimenting with serial dilutions of the concentrated antibody. For example, a common approach includes starting with the dilution recommended by the manufacturer with one dilution concentration above and one dilution concentration below. For example, if the recommended dilution is 1:400, then testing serial dilutions of 1:200, 1:400, and 1:800 may show optimal signal to background staining on one of the dilutions.

Methods for optimizing incubation time and/or incubation temperature to minimize background staining in situ are known in the art. In some embodiments, the sample may incubated with a probe for about 60 minutes to about 120 minutes, about 60 minutes to about 110 minutes, about 60 minutes to about 100 minutes, about 60 minutes to about 90 minutes, or about 60 minutes to about 80 minutes. In some embodiments, the sample may incubated with a probe overnight. In some embodiments, the sample may incubated with the probe at room temperature. In some embodiments, the sample may incubated with a probe at a temperature of about 4° C. to about 35° C., about 4° C. to about 25° C., about 4° C. to about 20° C., about 4° C. to about 10° C., about 10° C. to about 25° C., about 10° C. to about 20° C., about 10° C. to about 15° C., about 35° C. to about 50° C., about 35° C. to about 45° C., about 35° C. to about 40° C., about 40° C. to about 50° C., about 40° C. to about 45° C., or about 45° C. to about 50° C.

In some embodiments, the sample may be washed, e.g., with a washing buffer, after probe incubation is completed. In some embodiments, the sample may be washed in around 3 distinct changes of washing buffer. In some embodiments, the washing buffer may contain around 0.1% TBS-Tween. In some embodiments, the sample may be washed using a different reaction container for each wash. In some embodiments, the sample is washed for a total of about 5 minutes to about 20 minutes, about 9 minutes to about 20 minutes, about 10 minutes to about 20 minutes, about 11 minutes to about 20 minutes, about 12 minutes to about 20 minutes, about 13 minutes to about 20 minutes, or about 15 minutes to about 20 minutes.

In embodiments, controls may be included. For example, a tissue known to express the antigen may be used as a positive control. In embodiments, a tissue known not to express the antigen may be used as a negative control. In embodiments, a tissue may be probed in the same way, albeit with the omission of the target-specific probe, as a negative control.

In embodiments, the method includes binding a first probe to a first protein and binding a second probe to a second, different, protein, wherein the first probe includes a specific binding reagent attached to a first oligonucleotide including a first barcode sequence (e.g., a template polynucleotide described herein), and the second probe includes a specific binding reagent attached to a second oligonucleotide including a second barcode sequence (e.g., a template polynucleotide described herein). In embodiments, the method includes extending the first oligonucleotide including the first barcode sequence (e.g., a template polynucleotide described herein). In embodiments, the method includes ligating the first oligonucleotide including the first barcode sequence (e.g., a template polynucleotide described herein). In embodiments, the method includes forming a circular oligonucleotide including the first oligonucleotide including the first barcode sequence (e.g., a template polynucleotide described herein). In embodiments, the method includes extending the second oligonucleotide including the second barcode sequence (e.g., a template polynucleotide described herein). In embodiments, the method includes ligating the second oligonucleotide including the second barcode sequence (e.g., a template polynucleotide described herein). In embodiments, the method includes forming a circular oligonucleotide including the second oligonucleotide including the second barcode sequence (e.g., a template polynucleotide described herein).

In embodiments, the probe described herein is capable of binding to a biomolecule. In embodiments, the biomolecule is a lipid, carbohydrate, peptide, protein, or antigen binding fragment. In embodiments, the biomolecule is a lipid. In embodiments, the biomolecule is a carbohydrate. In embodiments, the biomolecule is a peptide. In embodiments, the biomolecule is a protein. In embodiments, the biomolecule is an antigen binding fragment. In embodiments, the biomolecule is an oligonucleotide. In embodiments, the target is a nucleic acid (e.g., the template polynucleotide described herein). In embodiments, the method further includes amplifying the nucleic acid sequence to generate amplification products. In embodiments, the method includes detecting the amplification products.

In embodiments, the target is a non-nucleic acid target. Non-nucleic acid targets include, but are not limited to, lipids, carbohydrates, peptides, proteins, glycoproteins, lipoproteins, phosphoproteins, acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In embodiments, the target is inside a cell or on a cell surface, such as a transmembrane analyte or one that is attached to the cell membrane. In embodiments, the target is an organelle (e.g., nuclei or mitochondria).

In embodiments, the probe described herein is capable of binding to an organelle. In embodiments, the organelle includes a nucleus, nucleoid, mitochondria, endoplasmic reticulum (ER), rough endoplasmic reticulum, smooth endoplasmic reticulum, Golgi apparatus, lysosomes, peroxisomes, ribosomes, cytoskeleton, microfilaments, intermediate filaments, microtubules, plasma membrane, chloroplasts (in plant cells and some protists), vacuoles, centrosomes and centrioles, nucleolus, nuclear envelope, nuclear pores, or transport vesicles. In embodiments, the biomolecule is in a cell. In embodiments, the biomolecule is on a cell (e.g., on the surface of a cell or tissue).

In embodiments, the probe is capable of binding to a protein. In embodiments, the first protein and the second protein are different proteins (e.g., a CD2 protein and a CD58 protein). In embodiments, the first protein and the second protein are the same protein. In embodiments, the first probe and the second probe contact the same protein (e.g., the first protein and the second protein are different epitopes on the same protein). In embodiments, the first probe and the second probe contact different proteins. In embodiments, the probe described herein contacts an agent (e.g., a small molecule or analyte). In embodiments, the agent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, or a sterol moiety.

In embodiments, the protein described herein is in or on a cell or tissue. In embodiments, the cell or tissue is fixed to a solid support. In embodiments, the cell is attached to a substrate. In embodiments, the cell is attached to the substrate via a bioconjugate reactive moiety. In embodiments, the cell or tissue sample is cleared (e.g., digested) of proteins, lipids, or proteins and lipids. In embodiments, the cell or tissue sample is processed according to a known technique in the art, for example CLARITY (Chung K., et al. Nature 497, 332-337 (2013)), PACT-PARS (Yang B., et al. Cell 158, 945-958 (2014).), CUBIC (Susaki E. A. et al. Cell 157, 726-739 (2014)., 18), ScaleS (Hama H., et al. Nat. Neurosci. 18, 1518-1529 (2015)), OPTIClear (Lai H. M., et al. Nat. Commun. 9, 1066 (2018)), Cₑ3D (Li W., et al. Proc. Natl. Acad. Sci. U.S.A. 114, E7321-E7330 (2017)), BABB (Dodt H. U. et al. Nat. Methods 4, 331-336 (2007)), iDISCO (Renier N., et al. Cell 159, 896-910 (2014)), uDISCO (Pan C., et al. Nat. Methods 13, 859-867 (2016)), FluoClearBABB (Schwarz M. K., et al. PLOS ONE 10, e0124650 (2015)), Ethanol-ECi (Klingberg A., et al. J. Am. Soc. Nephrol. 28, 452-459 (2017)), and PEGASOS (Jing D. et al. Cell Res. 28, 803-818 (2018)). In embodiments, the tissue includes liver tissue, kidney tissue, bone tissue, lung tissue, thymus tissue, adrenal tissue, skin tissue, bladder tissue, colon tissue, spleen tissue, or brain tissue.

In embodiments, the cell forms part of a tissue in situ. In embodiments, the cell is an isolated single cell. In embodiments, the cell is a prokaryotic cell. In embodiments, the cell is a eukaryotic cell. In embodiments, the cell is a bacterial cell, a fungal cell, a plant cell, or a mammalian cell. In embodiments, the cell is a stem cell. In embodiments, the stem cell is an embryonic stem cell, a tissue-specific stem cell, a mesenchymal stem cell, or an induced pluripotent stem cell. In embodiments, the cell is an endothelial cell, muscle cell, myocardial, smooth muscle cell, skeletal muscle cell, mesenchymal cell, epithelial cell; hematopoietic cell, such as lymphocytes, including T cell, e.g., (Th1 T cell, Th2 T cell, Th0 T cell, cytotoxic T cell); B cell, pre-B cell; monocytes; dendritic cell; neutrophils; or a macrophage. In embodiments, the cell is a stem cell, an immune cell, a cancer cell, a viral-host cell, or a cell that selectively binds to a desired target. In embodiments, the cell includes a T cell receptor gene sequence, a B cell receptor gene sequence, or an immunoglobulin gene sequence. In embodiments, the cell includes a Toll-like receptor (TLR) gene sequence. In embodiments, the cell includes a gene sequence corresponding to an immunoglobulin light chain polypeptide and a gene sequence corresponding to an immunoglobulin heavy chain polypeptide. In embodiments, the cell is a genetically modified cell.

In embodiments, the cell is a viral-host cell. A “viral-host cell” is used in accordance with its ordinary meaning in virology and refers to a cell that is infected with a viral genome (e.g., viral DNA or viral RNA). The cell, prior to infection with a viral genome, can be any cell that is susceptible to viral entry. In embodiments, the viral-host cell is a lytic viral-host cell. In embodiments, the viral-host cell is capable of producing viral protein. In embodiments, the viral-host cell is a lysogenic viral-host cell. In embodiments, the cell is a viral-host cell including a viral nucleic acid sequence, wherein the viral nucleic acid sequence is from a Hepadnaviridae, Adenoviridae, Herpesviridae, Poxviridae, Parvoviridae, Reoviridae, Coronaviridae, Retroviridae virus.

In embodiments, the cell is an adherent cell (e.g., epithelial cell, endothelial cell, or neural cell). Adherent cells are usually derived from tissues of organs and attach to a substrate (e.g., epithelial cells adhere to an extracellular matrix coated substrate via transmembrane adhesion protein complexes). Adherent cells typically require a substrate, e.g., tissue culture plastic, which may be coated with extracellular matrix (e.g., collagen and laminin) components to increase adhesion properties and provide other signals needed for growth and differentiation. Examples of such cells include, but are not limited to, cell lines derived from hematopoietic cells, and from the following cell lines: Colo205, CCRF-CEM, HL-60, K562, MOLT-4, RPMI-8226, SR, HOP-92, NCI-H322M, and MALME-3M. Non-limiting examples of adherent cells include DU145 (prostate cancer) cells, H295R (adrenocortical cancer) cells, HeLa (cervical cancer) cells, KBM-7 (chronic myelogenous leukemia) cells, LNCaP (prostate cancer) cells, MCF-7 (breast cancer) cells, MDA-MB-468 (breast cancer) cells, PC3 (prostate cancer) cells, SaOS-2 (bone cancer) cells, SH-SY5Y (neuroblastoma, cloned from a myeloma) cells, T-47D (breast cancer) cells, THP-1 (acute myeloid leukemia) cells, U87 (glioblastoma) cells, National Cancer Institute's 60 cancer cell line panel (NCI60), vero (African green monkey Chlorocebus kidney epithelial cell line) cells, MC3T3 (embryonic calvarium) cells, GH3 (pituitary tumor) cells, PC12 (pheochromocytoma) cells, dog MDCK kidney epithelial cells, Xenopus A6 kidney epithelial cells, zebrafish AB9 cells, and Sf9 insect epithelial cells. In embodiments, the cell is a neuronal cell, an endothelial cell, epithelial cell, germ cell, plasma cell, a muscle cell, peripheral blood mononuclear cell (PBMC), a myocardial cell, or a retina cell.

In embodiments, the cell is bound to a known antigen. In embodiments, the cell is a cell that selectively binds to a desired target, wherein the target is an antibody, or antigen binding fragment, an aptamer, affimer, non-immunoglobulin scaffold, small molecule, or genetic modifying agent. In embodiments, the cell is a leukocyte (i.e., a white-blood cell). In embodiments, leukocyte is a granulocyte (neutrophil, eosinophil, or basophil), monocyte, or lymphocyte (T cells and B cells). In embodiments, the cell is a lymphocyte. In embodiments, the cell is a T cell, an NK cell, or a B cell. In embodiments, the cell is an immune cell. In embodiments, the immune cell is a granulocyte, a mast cell, a monocyte, a neutrophil, a dendritic cell, or a natural killer (NK) cell. In embodiments, the immune cell is an adaptive cell, such as a T cell, NK cell, or a B cell. In embodiments, the cell includes a T cell receptor gene sequence, a B cell receptor gene sequence, or an immunoglobulin gene sequence. In embodiments, the plurality of target nucleic acids includes non-contiguous regions of a nucleic acid molecule. In embodiments, the non-contiguous regions include regions of a VDJ recombination of a B cell or T cell.

In embodiments, the cell is a cancer cell. In embodiments, the cancer is lung cancer, colorectal cancer, skin cancer, colon cancer, pancreatic cancer, breast cancer, cervical cancer, lymphoma, leukemia, or a cancer associated with aberrant K-Ras, aberrant APC, aberrant Smad4, aberrant p53, or aberrant TGFβ. In embodiments, the cancer cell includes a ERBB2, KRAS, TP53, PIK3CA, or FGFR2 gene. In embodiments, the cancer cell includes a HER2 gene. In embodiments, the cancer cell includes a cancer-associated gene (e.g., an oncogene associated with kinases and genes involved in DNA repair) or a cancer-associated biomarker. A “biomarker” is a substance that is associated with a particular characteristic, such as a disease or condition. A change in the levels of a biomarker may correlate with the risk or progression of a disease or with the susceptibility of the disease to a given treatment. In embodiments, the cancer is Acute Myeloid Leukemia, Adrenocortical Carcinoma, Bladder Urothelial Carcinoma, Breast Ductal Carcinoma, Breast Lobular Carcinoma, Cervical Carcinoma, Cholangiocarcinoma, Colorectal Adenocarcinoma, Esophageal Carcinoma, Gastric Adenocarcinoma, Glioblastoma Multiforme, Head and Neck Squamous Cell Carcinoma, Hepatocellular Carcinoma, Kidney Chromophobe Carcinoma, Kidney Clear Cell Carcinoma, Kidney Papillary Cell Carcinoma, Lower Grade Glioma, Lung Adenocarcinoma, Lung Squamous Cell Carcinoma, Mesothelioma, Ovarian Serous Adenocarcinoma, Pancreatic Ductal Adenocarcinoma, Paraganglioma & Pheochromocytoma, Prostate Adenocarcinoma, Sarcoma, Skin Cutaneous Melanoma, Testicular Germ Cell Cancer, Thymoma, Thyroid Papillary Carcinoma, Uterine Carcinosarcoma, Uterine Corpus Endometrioid Carcinoma, or Uveal Melanoma. In embodiments, the cancer-associated gene is a nucleic acid sequence identified within The Cancer Genome Atlas Program, accessible at online at cancer.gov/tcga.

In embodiments, the cell in situ is obtained from a subject (e.g., human or animal tissue). Once obtained, the cell is placed in an artificial environment in plastic or glass containers supported with specialized medium containing essential nutrients and growth factors to support proliferation. In embodiments, the cell is permeabilized and immobilized to a solid support surface (e.g., a microplate). In embodiments, the cell is permeabilized and immobilized within a well of the microplate. In embodiments, the cell is immobilized to a solid support surface (e.g., a well or a slide). In embodiments, the surface includes a patterned surface (e.g., suitable for immobilization of a plurality of cells in an ordered pattern. In embodiments, a plurality of cells is immobilized in wells of a microplate that have a mean or median separation from one another of about 10-20 μm. In embodiments, a plurality of cells is immobilized in wells of a microplate that have a mean or median separation from one another of about 10-20; 10-50; or 100 μm. In embodiments, a plurality of cells is arrayed on a substrate (e.g., a solid support). In embodiments, the solid support includes a functionalized glass surface or a functionalized plastic surface. In embodiments, the cell is attached to a multiwell container (e.g., a flow cell).

In embodiments, the cell is attached to the substrate (e.g., a solid support, such as a plastic or glass solid support) via a bioconjugate reactive linker. In embodiments, the cell is attached to the substrate via a specific binding reagent. In embodiments, the specific binding reagent includes an antibody, single-chain Fv fragment (scFv), antibody fragment-antigen binding (Fab), or an aptamer. In embodiments, the specific binding reagent includes an antibody, or antigen binding fragment, an aptamer, affimer, or non-immunoglobulin scaffold. In embodiments, the specific binding reagent is a peptide, a cell penetrating peptide, an aptamer, a DNA aptamer, an RNA aptamer, an antibody, an antibody fragment, a light chain antibody fragment, a single-chain variable fragment (scFv), a lipid, a lipid derivative, a phospholipid, a fatty acid, a triglyceride, a glycerolipid, a glycerophospholipid, a sphingolipid, a saccharolipid, a polyketide, a polylysine, polyethyleneimine, diethylaminoethyl (DEAE)-dextran, cholesterol, or a sterol moiety. Substrates may be prepared for selective capture of particular cells. For example, a substrate containing a plurality of bioconjugate reactive moieties or a plurality of specific binding reagents, optionally in an ordered pattern, contacts a plurality of cells. Only cells containing complementary bioconjugate reactive moieties or complementary specific binding reagents are capable of reacting, and thus adhering, to the substrate. In embodiments, the cell is immobilized to a substrate. Substrates can be two- or three-dimensional and can include a planar surface (e.g., a glass slide). A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites. In embodiments, the substrate includes a polymeric coating, optionally containing bioconjugate reactive moieties capable of affixing the sample. Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a sample. In embodiments, the substrate is not a flow cell. In embodiments, the substrate includes a polymer matrix material (e.g., polyacrylamide, cellulose, alginate, polyamide, cross-linked agarose, cross-linked dextran or cross-linked polyethylene glycol), which may be referred to herein as a “matrix”, “synthetic matrix”, “exogenous polymer” or “exogenous hydrogel”. In embodiments, a matrix may refer to the various components and organelles of a cell, for example, the cytoskeleton (e.g., actin and tubulin), endoplasmic reticulum, Golgi apparatus, vesicles, etc. In embodiments, the matrix is endogenous to a cell. In embodiments, the matrix is exogenous to a cell. In embodiments, the matrix includes both the intracellular and extracellular components of a cell. In embodiments, polynucleotide primers may be immobilized on a matrix including the various components and organelles of a cell. Immobilization of polynucleotide primers on a matrix of cellular components and organelles of a cell is accomplished as described herein, for example, through the interaction/reaction of complementary bioconjugate reactive moieties. In embodiments, the exogenous polymer may be a matrix or a network of extracellular components that act as a point of attachment (e.g., act as an anchor) for the cell to a substrate.

In embodiments, the methods are performed in situ on isolated cells or in tissue sections (alternatively referred to as a sample) that have been prepared according to methodologies known in the art. Methods for permeabilization and fixation of cells and tissue samples are known in the art, as exemplified by Cremer et al., The Nucleus: Volume 1: Nuclei and Subnuclear Components, R. Hancock (ed.) 2008; and Larsson et al., Nat. Methods (2010) 7:395-397, the content of each of which is incorporated herein by reference in its entirety. In embodiments, the cell is cleared (e.g., digested) of proteins, lipids, or proteins and lipids. In embodiments, the biological sample can be permeabilized using any of the methods described herein (e.g., using any of the detergents described herein, e.g., SDS and/or N-lauroylsarcosine sodium salt solution) before or after enzymatic treatment (e.g., treatment with any of the enzymes described herein, e.g., trypsin, proteases (e.g., pepsin and/or proteinase K)). In embodiments, the biological sample can be permeabilized by contacting the sample with a permeabilization solution. In some embodiments, the biological sample is permeabilized by exposing the sample to greater than about 1.0 w/v % (e.g., greater than about 2.0 w/v %, greater than about 3.0 w/v %, greater than about 4.0 w/v %, greater than about 5.0 w/v %, greater than about 6.0 w/v %, greater than about 7.0 w/v %, greater than about 8.0 w/v %, greater than about 9.0 w/v %, greater than about 10.0 w/v %, greater than about 11.0 w/v %, greater than about 12.0 w/v %, or greater than about 13.0 w/v %) sodium dodecyl sulfate (SDS) and/or N-lauroylsarcosine or N-lauroylsarcosine sodium salt. In some embodiments, the biological sample can be permeabilized by exposing the sample (e.g., for about 5 minutes to about 1 hour, about 5 minutes to about 40 minutes, about 5 minutes to about 30 minutes, about 5 minutes to about 20 minutes, or about 5 minutes to about 10 minutes) to about 1.0 w/v % to about 14.0 w/v % (e.g., about 2.0 w/v % to about 14.0 w/v %, about 2.0 w/v % to about 12.0 w/v %, about 2.0 w/v % to about 10.0 w/v %, about 4.0 w/v % to about 14.0 w/v %, about 4.0 w/v % to about 12.0 w/v %, about 4.0 w/v % to about 10.0 w/v %, about 6.0 w/v % to about 14.0 w/v %, about 6.0 w/v % to about 12.0 w/v %, about 6.0 w/v % to about 10.0 w/v %, about 8.0 w/v % to about 14.0 w/v %, about 8.0 w/v % to about 12.0 w/v %, about 8.0 w/v % to about 10.0 w/v %, about 10.0% w/v % to about 14.0 w/v %, about 10.0 w/v % to about 12.0 w/v %, or about 12.0 w/v % to about 14.0 w/v %) SDS and/or N-lauroylsarcosine salt solution and/or proteinase K (e.g., at a temperature of about 4° C. to about 35° C., about 4° C. to about 25° C., about 4° C. to about 20° C., about 4° C. to about 10° C., about 10° C. to about 25° C., about 10° C. to about 20° C., about 10° C. to about 15° C., about 35° C. to about 50° C., about 35° C. to about 45° C., about 35° C. to about 40° C., about 40° C. to about 50° C., about 40° C. to about 45° C., or about 45° C. to about 50° C.).

In embodiments, the cell is exposed to paraformaldehyde (i.e., by contacting the cell with paraformaldehyde). In embodiments, the cell is exposed to glutaraldehyde (i.e., by contacting the cell with glutaraldehyde). Any suitable permeabilization and fixation technologies can be used for making the cell available for the detection methods provided herein. In embodiments the method includes affixing single cells or tissues to a transparent substrate. Exemplary tissue includes those from skin tissue, muscle tissue, bone tissue, organ tissue and the like. In embodiments, the method includes immobilizing the cell in situ to a substrate and permeabilized for delivering probes, enzymes, nucleotides and other components required in the reactions. In embodiments, the cell includes many cells from a tissue section in which the original spatial relationships of the cells are retained. In embodiments, the cell in situ is within a Formalin-Fixed Paraffin-Embedded (FFPE) sample. In embodiments, the cell is subjected to paraffin removal methods, such as methods involving incubation with a hydrocarbon solvent, such as xylene or hexane, followed by two or more washes with decreasing concentrations of an alcohol, such as ethanol. The cell may be rehydrated in a buffer, such as PBS, TBS or MOPs. In embodiments, the FFPE sample is incubated with xylene and washed using ethanol to remove the embedding wax, followed by treatment with Proteinase K to permeabilized the tissue. In embodiments, the cell is fixed with a chemical fixing agent. In embodiments, the chemical fixing agent is formaldehyde or glutaraldehyde. In embodiments, the chemical fixing agent is glyoxal or dioxolane. In embodiments, the chemical fixing agent includes one or more of ethanol, methanol, 2-propanol, acetone, and glyoxal. In embodiments, the chemical fixing agent includes formalin, Greenfix®, Greenfix® Plus, UPM, CyMol®, HOPE®, CytoSkelFix™, F-Solv©, FineFIX®, RCL2/KINFix, UMFIX, Glyo-Fixx®, Histochoice®, or PAXgene®. In embodiments, the cell is fixed within a synthetic three-dimensional matrix (e.g., polymeric material). In embodiments, the synthetic matrix includes polymeric-crosslinking material. In embodiments, the material includes polyacrylamide, poly-ethylene glycol (PEG), poly(acrylate-co-acrylic acid) (PAA), or Poly(N-isopropylacrylamide) (NIPAM). In embodiments, the sample can be a biological sample selected from the group consisting of a freshly isolated sample, a fixed sample, a frozen sample, an embedded sample, a processed sample, or a combination thereof.

In embodiments the cell is lysed to release nucleic acid or other materials from the cells. For example, the cells may be lysed using reagents (e.g., a surfactant such as Triton™-X or SDS, an enzyme such as lysozyme, lysostaphin, zymolase, cellulase, mutanolysin, glycanases, proteases, mannase, proteinase K, etc.) or a physical lysing mechanism a physical condition (e.g., ultrasound, ultraviolet light, mechanical agitation, etc.). The cells may release, for instance, DNA, RNA, mRNA, proteins, or enzymes. The cells may arise from any suitable source. For instance, the cells may be any cells for which nucleic acid from the cells is desired to be studied or sequenced, etc., and may include one, or more than one, cell type. The cells may be for example, from a specific population of cells, such as from a certain organ or tissue (e.g., cardiac cells, immune cells, muscle cells, cancer cells, etc.), cells from a specific individual or species (e.g., human cells, mouse cells, bacteria, etc.), cells from different organisms, cells from a naturally occurring sample (e.g., pond water, soil, etc.), or the like. In some cases, the cells may be dissociated from tissue. In embodiments, the method does not include dissociating the cell from the tissue or the cellular microenvironment. In embodiments, the method does not include lysing the cell.

In embodiments, a permeabilization solution can contain additional reagents or a biological sample may be treated with additional reagents in order to optimize biological sample permeabilization. In some embodiments, an additional reagent is an RNA protectant. As used herein, the term “RNA protectant” typically refers to a reagent that protects RNA from RNA nucleases (e.g., RNases). Any appropriate RNA protectant that protects RNA from degradation can be used. A non-limiting example of an RNA protectant includes organic solvents (e.g., at least 60%, 65%, 70%, 75%, 80%, 85%, 90%, or 95% v/v organic solvent), which includes ethanol, methanol, propan-2-ol, acetone, trichloroacetic acid, propanol, polyethylene glycol, acetic acid, or a combination thereof. In embodiments, the RNA protectant includes ethanol, methanol and/or propan-2-ol, or a combination thereof. In embodiments, the RNA protectant includes RNAlater™ ICE (ThermoFisher Scientific). In embodiments, the RNA protectant includes a salt. The salt may include ammonium sulfate, ammonium bisulfate, ammonium chloride, ammonium acetate, cesium sulfate, cadmium sulfate, cesium iron (II) sulfate, chromium (III) sulfate, cobalt (II) sulfate, copper (II) sulfate, lithium chloride, lithium acetate, lithium sulfate, magnesium sulfate, magnesium chloride, manganese sulfate, manganese chloride, potassium chloride, potassium sulfate, sodium chloride, sodium acetate, sodium sulfate, zinc chloride, zinc acetate and zinc sulfate. In some embodiments, the biological sample is treated with one or more RNA protectants before, contemporaneously with, or after permeabilization.

In embodiments, the probe described herein binds to a carbohydrate, which is attached to the protein described herein. In embodiments, the probe described herein binds to a glycoprotein at the surface of the cell or tissue. In embodiments, the probe described herein binds to a glycolipid at the surface of the cell or tissue.

Post-translational modifications (PTMs) like glycosylation and phosphorylation play crucial roles in regulating protein function and cellular processes. Glycosylation, for example, can affect protein stability, folding, localization, and recognition by other molecules. By detecting and studying these modifications, scientists can gain insights into protein structure, function, and signaling pathways, helping us understand cellular processes, diseases, and develop targeted therapies. In embodiments, the probe described herein binds to a post-translational modification (e.g., phosphorylation analyte, methylation analyte, nitrosylation analyte, acetylation analyte, or glycosylation analyte). For example, phosphorylation is the addition of a phosphate group to a protein, such as phosphorylation of a serine residue in the protein kinase AMPK. In embodiments, the probe described herein binds to a phosphorylated amino acid. In embodiments, the probe described herein binds to a methylated amino acid. Methylation involves the addition of a methyl group to the protein described herein. One example is the methylation of histone proteins, which can regulate gene expression by acting as a signal for other proteins to bind and modify chromatin structure. In embodiments, the probe described herein binds to a nitrosylated amino acid. Nitrosylation is the addition of a nitric oxide group to the protein described herein. In cardiovascular disease, the nitrosylation of a specific cysteine residue in hemoglobin can affect its oxygen-carrying capacity. In embodiments, the probe described herein binds to an aceylated amino acid. Acetylation is the addition of an acetyl group to the protein described herein. Acetylation of histone tails can alter the chromatin structure, influencing gene expression. For instance, acetylation of histone H3 lysine 9 (H3K9) is associated with transcriptional activation. In embodiments, the probe described herein binds to an glycosylated amino acid. Glycosylation refers to the addition of a sugar molecule (oligosaccharide) to the protein described herein. One example is N-linked glycosylation, where sugars are added to a specific asparagine residue. Glycosylation of the glycoprotein erythropoietin is important for its stability and secretion.

In embodiments, the probe described herein includes a specific binding agent. In embodiments, the specific binding reagent is capable of binding to a cluster of differentiation (CD) marker, integrin, selectin, cadherin, cytokine receptor, chemokine receptor, Toll-like receptor (TLR), ion channel, transmembrane protein, lipoprotein, glycoprotein, cell surface protein, transport protein, or transcription factor. In embodiments, the specific binding agent is capable of binding an intracellular organelle. In embodiments, the intracellular organelle includes actin, centrosomes and centrioles, chloroplasts (in plant cells and some protists), cytoskeleton, endoplasmic reticulum, endosome, golgi apparatus, intermediate filaments, lysosome, microfilaments, microtubules, mitochondria, nuclear envelope, nuclear pores, nucleoid, nucleolus, nucleus, peroxisome, phosphatidylserine, plasma membrane, ribosomes, rough endoplasmic reticulum, smooth endoplasmic reticulum, transferrin receptor, transport vesicles, and/or vacuoles. In embodiments, the biomolecule specific binding agent is capable of binding to a biomolecule in the mitogen-activated protein kinase (MAPK) pathway, PI3K/AKT/mTOR pathway, Wnt/0-catenin pathway, intrinsic (mitochondrial) pathway, extrinsic (death receptor) pathway, caspase cascade, Notch signaling pathway, hedgehog signaling pathway, TGF-β (transforming growth factor Beta) pathway, JAK/STAT pathway, G-protein coupled receptor (GPCR) pathway, calcium signaling pathway, glycolysis, citric acid cycle (Krebs Cycle), oxidative phosphorylation, lipid metabolism pathway, amino acid metabolism, Toll-like receptor (TLR) pathway, NF-κB signaling pathway, complement pathway, nucleotide excision repair (NER), base excision repair (BER), mismatch repair (MMR), cyclin-dependent kinase (CDK) pathway, Rb (retinoblastoma) pathway, p53 pathway, unfolded protein response (UPR), heat shock response pathway, oxidative stress pathway, BMP (bone morphogenetic protein) pathway, FGF (fibroblast growth factor) pathway, Sonic Hedgehog pathway, neurotrophin signaling pathway, synaptic transmission pathway, axon guidance pathways, insulin signaling pathway, thyroid hormone pathway, steroid hormone pathway, VEGF (vascular endothelial growth factor) pathway, DNA methylation pathway, histone modification pathway, or angiogenesis. In embodiments, the biomolecule specific binding agent is capable of binding to a biomolecule on the surface of or in a B cell, Mature B Cell, Follicular B cell, Marginal Zone B cell, Short lived plasma cell, Memory B cell, Long lived plasma cell, B1 cell, Breg, Germinal Center B cell, Macrophage, Monocyte, M1 macrophage, M2 macrophage, Dendritic Cell, Plasmacytoid dendritic cell, Monocyte-derived dendritic cell, T cell, T Follicular Helper, Th1, Th2, Th9, Th17, Th22, Treg, platelet (activated), platelet (rested), natural killer cell, neutrophil, basophil, eosinophil, mast cell, astrocyte, neuron, glial cell, lymphocyte, myeloid cell, granulocytes, neural cells, stem cells, endothelial cells, epithelial cells, mesenchymal stem cell, hematopoietic stem cell, embryonic stem, stromal cell, erythrocyte, fibroblast, or apoptotic cell.

In embodiments, the specific binding reagent is an antibody, single-chain Fv fragment (scFv), antibody fragment-antigen binding (Fab), lectin, affimer, or an aptamer. The selection of the specific binding reagent depends on the intended target biomolecule. In embodiments, the specific binding reagent binds to a target molecule having a K_{D} is less than 10⁻⁶ M, less than 10⁻⁷ M, less than 10⁻⁸ M, less than 10⁻⁹ M, less than 10⁻⁹ M, less than 10⁻¹¹ M, or less than about 10⁻¹² M or less. The K_{D} represents the concentration of ligand (e.g., target molecule) at which half of the binding sites of the binding partner (e.g., specific binding reagent) are occupied. A lower K_{D} indicates stronger binding affinity, as less ligand is required to achieve 50% occupancy. In embodiments, the K_{D} is 10⁻⁶ M to 10⁻¹² M. In embodiments, the K_{D} is 10⁻⁸ M to 10⁻¹² M. In embodiments, the K_{D} is 10⁻⁹ M to 10⁻¹² M. In embodiments, the K_{D} is 10⁻¹⁰ M to 10⁻¹² M. In embodiments, the K_{D} is 10⁻⁸ M to 10⁻¹⁰ M. Non-limited examples of target biomolecules and specific binding reagents is provided in the Target Table:

Target Table:

| Target Biomolecule | Specific Binding Reagent |
|---|---|
| Protein | Antibodies, aptamers, RNAs, modified bases |
| Carbohydrate | Lectins |
| Peptide | Antibodies or aptamers |
| Small molecule(s) | Proteins or aptamers |
| Lipids | Proteins or aptamers |
| Biotinylated biomolecule | Avidin (e.g., streptavidin) |

In embodiments, each probe is an antibody, an antibody fragment, an affimer, an aptamer, or a nucleic acid. The antibodies used for the probes may be polyclonal or monoclonal antibodies, or fragments of antibodies. Further, the antibodies forming the protein-probe complex may have the same binding specificity or differ in their binding specificities. Further contemplated herein is the use of variations, e.g., that are described in WO2012/104261, which is incorporated herein by reference in its entirety. For example, the probes may each be linked to their respective antibody at the 5′ end, or one probe may be linked at the 5′ end and the other at the 3′ end.

A probe is defined herein as an entity including an analyte-binding domain specific for a biomolecule (e.g., a protein), and a nucleic acid domain (e.g., a probe oligonucleotide). By “specific for biomolecule” is meant that the biomolecule-binding domain specifically recognizes and binds a particular target biomolecule, i.e., it binds its target biomolecule with higher affinity than it binds to other biomolecules or moieties. In embodiments, the biomolecule-binding domain is an antibody, in particular a monoclonal antibody. Antibody fragments or derivatives of antibodies including the biomolecule-binding domain are also suitable for use as the biomolecule binding domain. Examples of such antibody fragments or derivatives include Fab, Fab′, F(ab′)2 and scFv molecules.

A Fab fragment consists of the antigen-binding domain of an antibody. An individual antibody may be seen to contain two Fab fragments, each consisting of a light chain and its conjoined N-terminal section of the heavy chain. Thus, a Fab fragment contains an entire light chain and the VH and CH1 domains of the heavy chain to which it is bound. Fab fragments may be obtained by digesting an antibody with papain.

F(ab′)2 fragments consist of the two Fab fragments of an antibody, plus the hinge regions of the heavy domains, including the disulfide bonds linking the two heavy chains together. In other words, a F(ab′)2 fragment can be seen as two covalently joined Fab fragments. F(ab′)2 fragments may be obtained by digesting an antibody with pepsin. Reduction of F(ab′)2 fragments yield two Fab′ fragments, which can be seen as Fab fragments containing an additional sulfhydryl group which can be useful for conjugation of the fragment to other molecules. ScFv molecules are synthetic constructs produced by fusing together the variable domains of the light and heavy chains of an antibody. Typically, this fusion is achieved recombinantly, by engineering the antibody gene to produce a fusion protein which includes both the heavy and light chain variable domains.

The nucleic acid domain of a probe may be a DNA domain or an RNA domain. Preferably it is a DNA domain. In embodiments, the nucleic acid domains (e.g., probe oligonucleotide) of the probes are designed to hybridize to another oligonucleotide molecule. In embodiments the probe oligonucleotides of the probes are single-stranded. In other embodiments, the probe oligonucleotides of the probes are partially single-stranded, including both a single-stranded portion and a double-stranded portion.

In embodiments, specific binding entails a binding affinity, typically expressed as a KD (such as a KD measured by surface plasmon resonance at an appropriate temperature, such as 37° C.). In embodiments, the KD of a specific binding interaction is less than about 100 nM, 50 nM, 10 nM, 1 nM, 0.05 nM, or lower. In embodiments, the KD of a specific binding interaction is about 0.01-100 nM, 0.1-50 nM, or 1-10 nM. In embodiments, the KD of a specific binding interaction is less than 10 nM. The binding affinity of an antibody can be readily determined by one of ordinary skill in the art (for example, by Scatchard analysis). A variety of immunoassay formats can be used to select antibodies specifically immunoreactive with a particular antigen. For example, solid-phase ELISA immunoassays are routinely used to select monoclonal antibodies specifically immunoreactive with an analyte. See Harlow and Lane, ANTIBODIES: A LABORATORY MANUAL, Cold Springs Harbor Publications, New York, (1988) for a description of immunoassay formats and conditions that can be used to determine specific immunoreactivity. Typically, a specific or selective reaction will be at least twice background signal to noise and more typically more than 10 to 100 times greater than background.

### EXAMPLES

### Example 1. Two Color Sequencing Using Orthogonal Cleavable Linkers

Among numerous commercially available high throughput sequencing platforms, some platforms rely on using nucleobase-specific fluorescently labelled nucleotides and detecting the fluorescence emissions associated with each nucleotide type (e.g., adenine, thymine, guanine, and cytosine) to determine the sequence of a target polynucleotide. During a sequencing cycle, the fluorescence signal is recorded when a fluorescently-excited labelled nucleotide is incorporated into a growing strand complementary to the template polynucleotide strand that is immobilized onto a flow cell. The optical components of fluorescence-based sequencing platforms employ either four detection channels or two detection channels to detect the fluorescence signals following nucleotide incorporation to facilitate base calling. Platforms leveraging four detection channels (referred herein as 4-color or “4-channel”) utilize four different wavelength emission filters to detect the unique fluorescence emissions from each of the four fluorophores, wherein each detection channel corresponds to one of the four different fluorescent dyes used for labeling nucleotides during the sequencing process. These fluorescent dyes are typically represented by four different colors (e.g., red, green, blue, and yellow), enabling the identification of the four nucleotide bases (A, T, C, and G). These 4-color sequencing platforms have been implemented for broad applications in genomics but suffer from higher costs compared to platforms that use two detection channels (referred herein as 2-color or 2-channel). Since a four-color platform uses four distinct fluorescent dyes, the optical detection system needs to be capable of detecting and differentiating between four different wavelengths of light emitted by the different dyes. This requires more advanced optics and detectors capable of multicolor imaging. On the other hand, a two-color platform requires a simpler optical detection system that can discriminate between only two wavelengths of light emitted by the two different fluorescent dyes used.

Sequencing platforms that rely on two detection channels aim to overcome challenges identified in the 4-color sequencing platforms. Instead of acquiring four images in four distinct channels, the 2-color sequencing platforms require merely two detection channels to determine all four bases. For example, one implementation of a 2-color sequencing platform, the first nucleotide type (e.g., cytosine nucleotide) is detected using an emission wavelength filter for the emission of fluorophore A; the second nucleotide type (e.g., thymine nucleotide) is detected using an emission wavelength filter for the emission of fluorophore B, where fluorophore B is spectrally distinct from fluorophore A; the third nucleotide type (e.g., adenine nucleotide) is detected by the emission of both fluorophores A and B; the last nucleotide type (e.g., guanine nucleotide) is detected by the absence of fluorescent signal (see, for example the two-color sequencing systems and methods as described in U.S. Pat. Nos. 9,453,258 and/or 8,617,811, each of which are incorporated herein by reference). The use of two spectrally distinct fluorophores, instead of four fluorophores, reduces the complexity of nucleobase discrimination following image acquisition, and may improve the efficiency of base calling. Despite improvements in speed afforded by the aforementioned 2-color sequencing platforms, one key drawback stems from the reliance of the absence of fluorescence signal for base calling of one of the nucleotides. The lack of fluorescence signal could also signify the lack of extension at a given cluster during a sequencing cycle as the lack of extension (i.e., failure to incorporate an unlabeled nucleotide) would also result in a lack of fluorescence signal, which could result in errors in base calling or overcalling of the unlabeled bases (De-Kayne et al. Mol Ecol Resour. 2021 April; 21(3):653-660, which is incorporated herein by reference). Additionally, Arora et al. performed whole genome sequencing of three cancer cell lines using a 4-color sequencing platform, Illumina® HiSeq X™ Ten, and a 2-color sequencing platform, Illumina® NovaSeq™ 6000 (Arora et al. Sci Rep. 2019 Dec. 13; 9(1):19123, which is incorporated herein by reference). Using the NovaSeq™ sequencing system, the emission corresponding to the cytosine nucleotide is detected using a red wavelength filter, the emission corresponding to the thymine is detected using a green wavelength filter, emission detected by both the red and green wavelength filters correspond to the adenine nucleotide, and the lack of emission is used to call for the guanine nucleotide. Relative to the sequencing data obtained from the 4-color sequencing platform, Arora et al. observed higher frequencies in base calling of the guanine nucleotide and higher occurrences of thymine to guanine nucleotide mismatches in the sequencing data obtained from the 2-color sequencing platform. Arora et al. postulated that these observations resulted from artifactual base calling due to using the absence of signal to call for the guanine nucleotide. As described supra, the ambiguity associated with incorporating an unlabeled nucleotide coupled with the reliance of the absence of fluorescence signal for base calling could lead to erroneous base calling, and these key features remain as important limitations to current commercially available 2-color sequencing platforms.

Provided herein, inter alia, are novel compounds and methods useful for an improved sub-four color (e.g., a 2-color) sequencing method. In embodiments, and as described herein, the compounds and methods utilize four fluorescently labeled nucleotides that are differentiated by orthogonal cleavable linkers to facilitate base calling in a 2-color sequencing system. As described herein, pluralities of two sets of fluorescently labeled nucleotides, wherein each set contains two of the four nucleotides types (e.g., one set includes A and T, the other set includes C and G). Each nucleotide is conjugated to one of the two spectrally distinct fluorophores via the same cleavable linker type (i.e., a cleavable linker capable of being cleaved under similar or identical conditions). Through the use of orthogonal cleavable linkers, the cleavable linker attached to each fluorescently labeled nucleotide within a given set will be removed under identical cleaving conditions, but different cleaving conditions are used to remove the cleavable linkers belonging to the first and second set.

FIG. 1 illustrates an example of two potential sets of fluorescently labelled nucleotides, where each set differ from the other by the orthogonal cleavable linkers attached between the nucleobase and the fluorophore moiety (shown as the star shape). It is understood that any configuration of the two sets is contemplated herein, for example dATP and dCTP may form the first set, or alternatively dATP and dTTP form the first set. The differences between each set relates to the cleavable linker type. As depicted in FIG. 1, the first set includes an adenine nucleotide covalently labeled with Dye 1 and a guanine nucleotide covalently labeled with Dye 2. The adenine nucleotide and guanine nucleotide are attached to the respective fluorophore moieties via cleavable linker 1. The second set includes a thymine nucleotide covalently labeled with Dye 1 and a cytosine nucleotide covalently labeled with Dye 2. The thymine nucleotide and cytosine nucleotide are attached to the respective fluorophore moieties via cleavable linker 2. The illustrations of the modified nucleotides include a cartoon block on the lower left side of each nucleotide, representing a 3′ reversible terminator. A reversible terminator serves as a temporary stopper for DNA synthesis, allowing the sequencing machinery to determine the identify of the incorporated base before continuing with the next cycle. After the base identification, the reversible terminator is chemically cleaved (e.g., contacting the incorporated nucleotide with a cleaving agent) thereby removing the blocking group. In embodiments, the reversible terminator and the cleavable linker are cleaved under identical conditions. This cleavage reaction allows the DNA synthesis to continue with the next nucleotide incorporation. The reversible nature of the terminator enables repeating this process for multiple cycles, facilitating high-throughput sequencing.

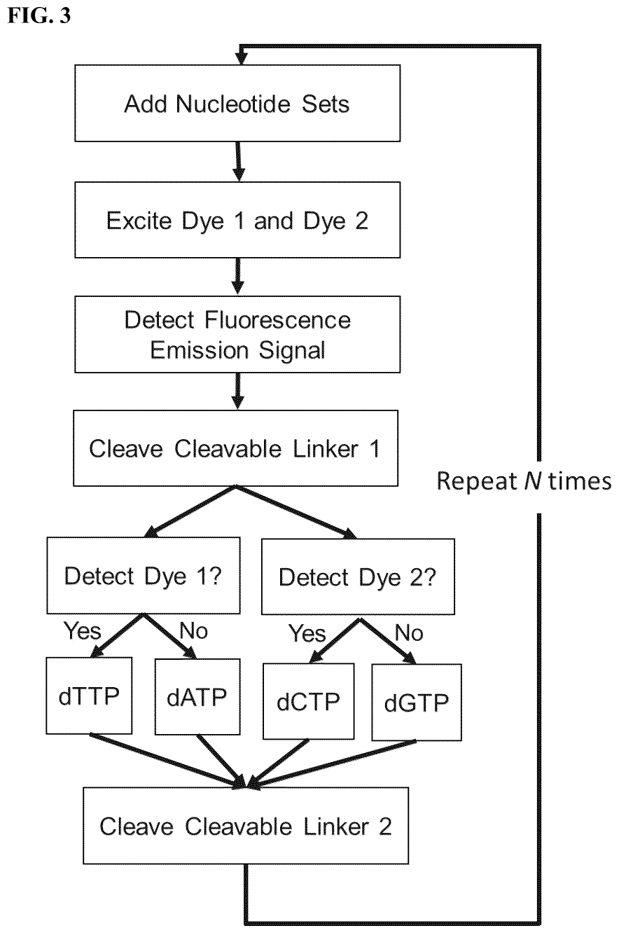

Shown in FIG. 2 is a table outlining the dye detection events following successive rounds of imaging and cleaving using the nucleotide sets illustrated in FIG. 1. For example, following incorporation and excitation an image is obtained in both channels and the fluorescence emission signal deriving from Dye 1 of the adenine and the thymine nucleotides as well as the signal from Dye 2 of the guanine and cytosine nucleotides may be detected during the first image event. Though not explicitly shown in the workflow, it is understood that one of the corresponding nucleotides is incorporated into a complementary strand during a sequencing cycle. The possibility of detecting Dye 1 and Dye 2 is depicted by the presence of all the stars in the first row of FIG. 2. After the cleavage of cleavable linker 1, the fluorescence emission signal of Dye 1 from the adenine nucleotide and the fluorescence emission signal of Dye 2 from the guanine nucleotide would be extinguished if either of these nucleotides were incorporated into the complementary strand during the sequencing cycle. Any fluorescence signal detected from Dye 1 or Dye 2 following the cleavage of cleavable linker 1 would derive from the thymine nucleotide and cytosine nucleotide, respectively, as these nucleotides are attached to the respective fluorophore moieties via cleavable linker 2. Following the cleavage of cleavable linker 2, the fluorescence emission signal of Dye 1 or Dye 2 will also be removed and a new sequencing cycle may then occur. This process occurs iteratively, as illustrated in FIG. 3, to facilitate base calling using the fluorescently labeled nucleotides described herein with repeated steps of excitation, detection of fluorescence emission signal, cleavage of cleavable linker 1, detection of remaining or the absence of a fluorescent emission signal. Cleavage of cleavable linker 2 resets the cycle.

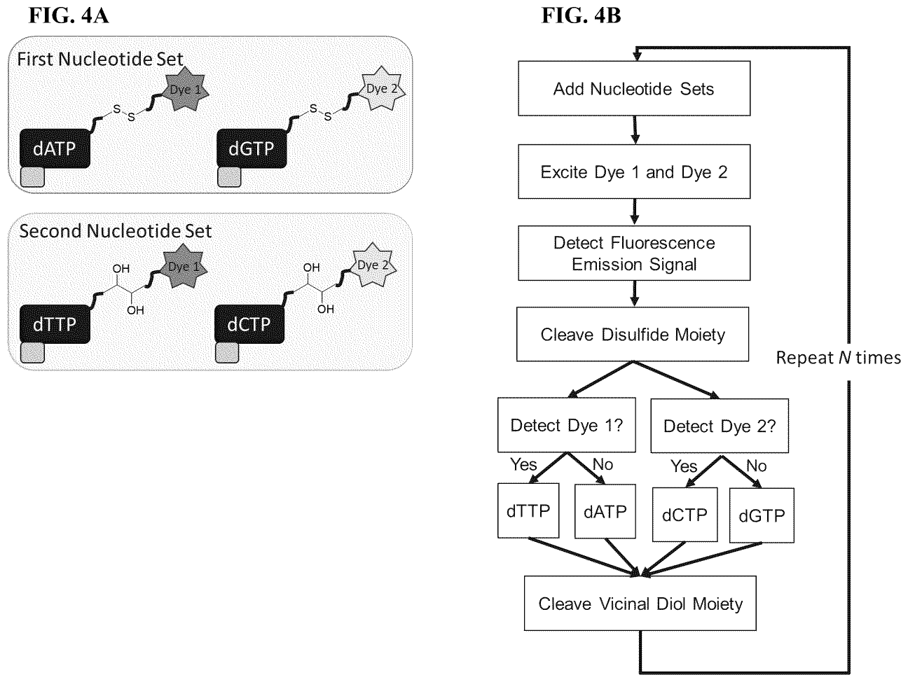

Examples of orthogonal cleavable linkers are contemplated herein. For example, the first set of fluorescently labelled nucleotides includes cleavable linkers with a disulfide moiety, while the nucleotides from the second set are covalently attached to the respective fluorophore moieties via covalent linkers containing a vicinal diol (FIG. 4A). Using these orthogonal cleavable linkers, the fluorophore moieties from the first set of nucleotides are removed following the cleavage of the disulfide moieties in the presence of a reducing agent (e.g., THPP), while the fluorophore moieties from the second set of nucleotides are removed following the cleavage of the vicinal diols moieties in the presence of an oxidizing agent (e.g., NaIO₄). As shown in FIG. 4B, the identity of the incorporated base is determined from the combination of the detection of the fluorescence emission signal prior to the cleavage of the disulfide moiety present in the cleavable linkers of the first set of nucleotides and the absence of the signal thereof.

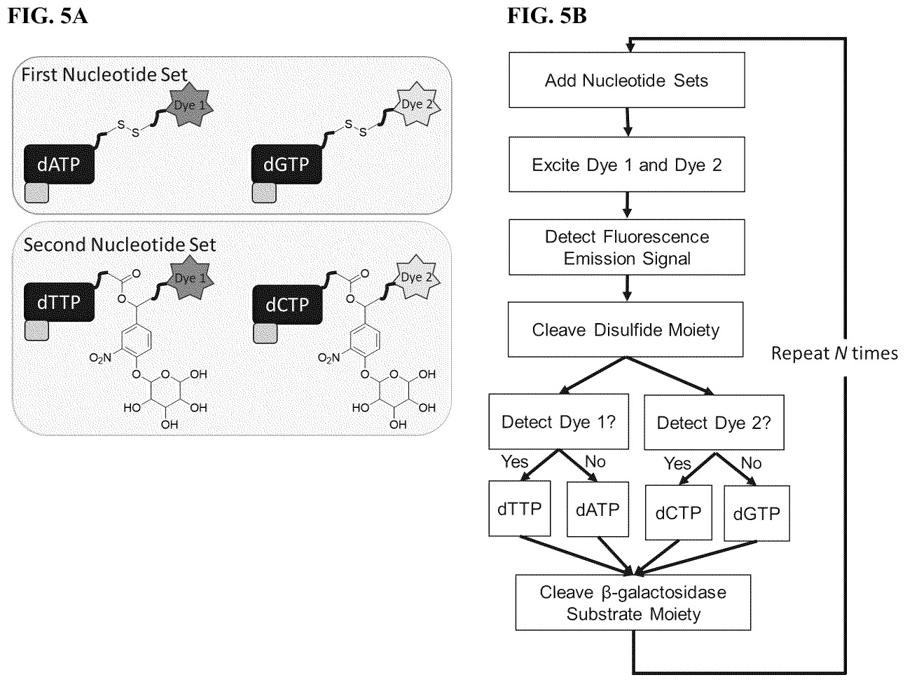

Alternative orthogonal cleavable linkers are contemplated herein. As shown in FIG. 5A, the fluorescently labelled nucleotides of the first set harbor a disulfide moiety in the cleavable linker, while the fluorescently labelled nucleotides of the second set include a β-galactosidase substrate moiety in the cleavable linker. The use of these cleavable linkers benefits from the orthogonality afforded by the reductant-labile disulfide moiety and the β-galactosidase sensitive substrate. Similar to the process shown in FIG. 4B, the identity of the incorporated base is determined from the combination of the detection of the fluorescence emission signal prior to the cleavage of the disulfide moiety present in the cleavable linkers of the first set of nucleotides and the absence of the signal thereof (FIG. 5B).

### Example 2. Two Color Sequencing in a Flow Cell

A significant advantage provided from the nucleotides and methods described herein over conventional methods of 2-color sequencing derives from the reliance of both the detection of a fluorescence emission signal from each nucleotide following the sequential addition of orthogonal cleaving agents.

Current SBS platforms utilize clonal amplification of the initial template library molecules to create clusters (i.e., polonies) on a flow cell, each containing 100s to 10,000s of forward and reverse copies of an initial template library molecule, to increase the signal-to-noise ratio because the systems are not sensitive enough to detect the extension of one base at the individual DNA template molecule level. Flow cells provide a convenient format for housing an array of clusters, in particular when subjected to an SBS or other detection technique that involves repeated delivery of reagents in cycles. For example, bridge amplification methods allow amplification products to be immobilized on a solid support in order to form arrays comprised of colonies (or “clusters”) of immobilized nucleic acid molecules. Each cluster or colony on such an array is formed from a plurality of identical immobilized polynucleotide strands and a plurality of identical immobilized complementary polynucleotide strands. The products of solid-phase amplification reactions are referred to as “bridged” structures when formed by annealed pairs of immobilized polynucleotide strands and immobilized complementary strands, both strands being immobilized on the solid support at the 5′ end, preferably via a covalent attachment to the surface of the flow cell.

To initiate a first SBS cycle, labeled nucleotides and a DNA polymerase in a buffer can be flowed into/through a flow cell that houses the surface-immobilized clusters. Extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments that have been attached at different locations of an array can be subjected to an SBS technique as described herein under conditions where events occurring for different templates can be distinguished due to their location in the array. For example, the sequencing step may include annealing and extending a sequencing primer to incorporate a detectable label that indicates the identity of a nucleotide in the target polynucleotide, detecting the detectable label, and repeating the extending and detecting steps. In embodiments, the nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 10,738,072 and/or 11,174,281. Once such a modified nucleotide has been incorporated into the growing polynucleotide chain complementary to the region of the template being sequenced, there is no free 3′-OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Washes can be carried out between the various delivery steps as needed. The cycle can then be repeated N times to extend the primer by N nucleotides, thereby detecting a sequence of length N. Example SBS procedures, fluidic systems and detection platforms that can be readily adapted for use with an array produced by the methods of the present disclosure are described, for example, in Bentley et al., Nature 456:53-59 (2008), US Patent Publication 2018/0274024, WO 2017/205336, US Patent Publication 2018/0258472, each of which are incorporated herein in their entirety for all purposes.

The initiation point for the first sequencing reaction is provided by annealing a sequencing primer complementary to an immobilized strand, wherein the strand is covalently attached to a solid support. In the presence of an enzyme (e.g., a DNA polymerase), the two sets of nucleotides (e.g., the labeled nucleotides illustrated in FIG. 1) contact the sequencing primer. Under suitable conditions, one of the four nucleotides is incorporated into the sequencing primer. To detect the identity of the incorporated nucleotide, an excitation light (e.g., a laser) is directed toward the nucleotide which excites the attached fluorophore. A first image is obtained and the first cleavable linker is cleaved, thereby removing any fluorophores attached to a nucleotide via the first cleavable linker type. A second image is obtained to determine the identity of the incorporated nucleotide. Table 1 provides a representation of data collected for two sequencing cycles using the nucleotides described in FIG. 1 and the workflow of FIG. 3.

TABLE 1
Base calling using fluorescently labelled nucleotides for
2-color sequencing for two sequencing cycles. A “1”
in the table indicates the fluorescent emission is detected,
whereas a “0” means no emissions are detected. “CL”
is an abbreviation for cleavable linker.

| | | |
|---|---|---|
| Image Event | Image Event | Cleave |
| (Prior to Cleaving) | (Post Cleave CL1) | CL2 |

Cycle 1

| | | | |
|---|---|---|---|
| Probe set 1 | | | |
| dATP-CL1-Dye 1 | 0 | 0 | 0 |
| dGTP-CL1-Dye 2 | 0 | 0 | 0 |
| Probe set 2 | | | |
| dTTP-CL2-Dye 1 | 1 | 1 | 0 |

Cycle 2

| | | | |
|---|---|---|---|
| Probe set 1 | | | |
| dATP-CL1-Dye 1 | 0 | 0 | 0 |
| dGTP-CL1-Dye 2 | 1 | 0 | 0 |
| Probe set 2 | | | |
| dTTP-CL2-Dye 1 | 0 | 0 | 0 |
| dCTP-CL2-Dye 2 | 0 | 0 | 0 |

During the first sequencing cycle, all four nucleotides are added to the flow cell. The fluorescence emission signal of Dye 1 is detected prior to the addition of the cleaving agent. The fluorescence emission signal of Dye 1 persists following the cleavage of cleavable linker 1. Based on the combination of detection events, i.e., the combination of detecting Dye 1 prior to the addition of cleaving agents and following the addition of cleaving agents enables the identification of thymine as the incorporated base during the first sequencing cycle. The second cleavable linker is cleaved enabling a second sequencing cycle.

During the second sequencing cycle, all four nucleotides are added to the flow cell. The fluorescence emission signal of Dye 2 is detected prior to the addition of the cleaving agent. A cleaving agent is added, cleaving the first cleavable linker, and a second image event occurs. The signal associated with Dye 2 is not detected. This combination of detecting Dye 2, followed by detection of its absence enables guanine to be inferred as the incorporated nucleotide. The application of the nucleotides described herein enables a bimodal strategy of detection of fluorescence and the absence thereof to ensure accurate base calling for incorporation events as fluorescence emission signal corresponding to all four nucleotides are measured.

### Example 3. Applications of Two Color Sequencing for Spatial Transcriptomics

One key influencing factor in the pathophysiological development of a disease stems from the aberrant gene and protein expression of disease-relevant genes and proteins along with the spatial heterogeneity in their abundance and distribution among cells and tissues. Spatial biology techniques, such as in situ sequencing, enables the scrutiny of disease-relevant biomolecules (such as lipids, carbohydrates, nucleic acids, and/or proteins) in the original context of intact tissue, which enables the evaluation of these macromolecules in relation to the tissue architecture and cellular microenvironment, both of which are governed by the intracellular and intercellular communication in situ.

Provided herein are compositions and methods for detecting biomolecules (e.g., nucleic acids) in tissue sections in situ. Tissue sections may be manipulated using methods and techniques known in the art to for in situ transcriptomics workflows (see, e.g., U.S. Pat. No. 11,891,656, which is incorporated herein by reference in its entirety). For example, a tissue section including a nucleic acid of interest (e.g., the mRNA transcript of the oncogene ERBB2) is detected in a tissue section adhered onto a functionalized glass slide described herein. Detection of the nucleic acid of interest requires the development of a detection agent, such as an oligonucleotide probe or padlock probe, with a sequence capable of hybridizing with the nucleic acid of interest to facilitate its detection in situ (e.g., oligonucleotide label). In embodiments, 1, 2, 3, 4, 5, 6, 7, 8, 9, 10, 11, 12, 13, 14, 15, 16, 17, 18, 19, or 20 unique padlock probes were used to target each gene. In embodiments, 3 unique padlock probes were used to target each gene. In embodiments, 5 unique padlock probes were used to target each gene. In embodiments, 7 unique padlock probes were used to target each gene. In embodiments, 9 unique padlock probes were used to target each gene. In embodiments, 11 unique padlock probes were used to target each gene. In embodiments, 12 unique padlock probes were used to target each gene. The determination of the sequence of the oligonucleotide label and its association to the nucleic acid of interest is made a priori, and the oligonucleotide label the is capable of being detected by various methods. In embodiments, the oligonucleotide label is amplified prior to detection to boost its signal for detection. In embodiments, the padlock probe harboring the oligonucleotide label is ligated and amplified prior to detection. In embodiments, the mode of detection is by sequencing-by-synthesis, where the sequence of the oligonucleotide label is detected and used to associate and identify the nucleic acid of interest in the tissue section following bioinformatic analyses. In embodiments, sequencing-by-synthesis is performed using the methods and compositions described herein (e.g., see Examples 1 and 2). In embodiments, the template polynucleotide as described herein includes the sequence of the nucleic acid of interest or complement thereof (e.g., a sequence of an endogenous nucleic acid molecule, which may be amplified via RCA). In embodiments, the template polynucleotide as described herein includes the sequence of the oligonucleotide label or complement thereof. In embodiments, the template polynucleotide as described herein includes the sequence of the nucleic acid of interest or complement thereof and the sequence of the oligonucleotide label or complement thereof.

### Example 4. Applications of Two Color Sequencing for Combined Spatial Transcriptomics and Proteomics

We proceeded to adapt the workflow described supra to enable a combined transcriptomic and proteomics readout using the same tissue attached to a solid support described herein (e.g., solid support including a functionalized glass slide described herein). Buccitelli et al. reported that transcriptomics and proteomics provide non-redundant readouts as mRNA levels detected from a transcriptomics readout do not invariably correlate with its protein levels (see, e.g., Buccitelli et. al. Nat Rev Genet. 2020 October; 21(10):630-644. doi: 10.1038/s41576-020-0258-4. Epub 2020 Jul. 24, which is incorporated herein by reference in its entirety). Different confounding factors, such as epigenetic changes to the transcriptional state of a gene, transcription rates, mRNA half-lives, whether proteins are transported to different cellular locations following their translation, and protein half-lives, could impact protein levels and/or cause deviations in correlations between the mRNA and protein levels for individual genes. As such, obtaining proteomics data and/or in combination with the transcriptomics data from the same tissue section provides immeasurable insight to unraveling the biological complexities of healthy and/or diseased cells within a tissue sample.

Provided herein are compositions and methods for in situ detection of nucleic acids and proteins in tissue sections. A combined transcriptomics and proteomics study was conducted using a solid support and nucleotides described herein to detect nucleic acids of interest and proteins of interest in a tissue section. In embodiments, the solid support is a flow cell that includes a two-lane configuration. In embodiments, the solid support is a flow cell that includes a four-lane configuration. Tissues were prepared for transfer to the solid support in deparaffinization and heat-induced antigen retrieval steps using techniques known in the art (see, e.g., PCT Publication WO2023076832A1). Following the transfer of the tissues onto the solid support, padlock probes targeting the target RNA transcripts were allowed to hybridize with the nucleic acids of interest. In embodiments, 3 unique padlock probes with a sequence capable of hybridizing with a nucleic acid of interest are used to facilitate its detection in situ. In embodiments, 7 unique padlock probes with a sequence capable of hybridizing with a nucleic acid of interest are used to facilitate its detection in situ. In embodiments, 12 unique padlock probes with a sequence capable of hybridizing with a nucleic acid of interest are used to facilitate its detection in situ. Following hybridization, the padlock probes targeting the nucleic acids of interest were ligated using SplintR® ligase and amplified using rolling circle amplification. Following amplification of the ligated padlock probes corresponding to the nucleic acids of interest, the tissue section was contacted with detection agents targeting proteins of interest. In embodiments, the detection agent is an antibody with an oligonucleotide label, where the determination of the sequence of the oligonucleotide label and its association to a protein of interest is made a priori. In embodiments, the oligonucleotide label is a padlock probe, where the sequence of the padlock probe and its association to a protein of interest is made a priori. Following the binding interaction between target-specific antibody and the protein of interest, padlock probes associated with the proteins of interest were ligated using SplintR® ligase and amplified using rolling circle amplification. Amplicons corresponding to the padlock probes targeting nucleic acids of interest and amplicons associated with proteins of interest were sequenced. In embodiments, tissue sample was further detected using fluorescent hematoxylin and eosin (H&E) staining. In embodiments, the template polynucleotide as described herein includes the sequence of the oligonucleotide label or complement thereof, wherein the determination of the sequence of the oligonucleotide label enables its association to the protein of interest. In embodiments, the template polynucleotide as described herein includes the sequence of the oligonucleotide label or complement thereof, wherein the determination of the sequence of the oligonucleotide label enables its association to the nucleic acid of interest.

Alternatively, or additionally, methods for the detection of proteins of interest without the detection of nucleic acids of interest were also contemplated. For a proteomics workflow, tissues were prepared for transfer to the solid support in deparaffinization and heat-induced antigen retrieval steps as described supra and contacted with detection agents targeting proteins of interest. In embodiments, the detection agent is an antibody with an oligonucleotide label, where the determination of the sequence of the oligonucleotide label and its association to the protein of interest is made a priori. In embodiments, the oligonucleotide label is a padlock probe, where the sequence of the padlock probe and its association to the protein of interest is made a priori. Following the binding interaction between target-specific antibody and the protein of interest, padlock probes associated with the proteins of interest were ligated using SplintR® ligase and amplified using rolling circle amplification. Amplicons associated with proteins of interest were sequenced. In embodiments, biomolecules and/or cellular structures of interest were further detected using fluorescent hematoxylin and eosin (H&E) staining. In embodiments, about 10, 11, 12, 13, 14, 15, 16, 17, 18, 19, 20, 21, 22, 23, 24, 25, 26, 27, 28, 29, 30, or more proteins of interest are detected. In embodiments, about 10 proteins of interest are detected. In embodiments, about 15 proteins of interest are detected. In embodiments, about 20 proteins of interest are detected.

### Example 5. Applications of Two Color Sequencing and Cell Paints

The detection and analysis of multiple biomolecules within the same cell or tissue section is crucial for understanding the phenotypic and functional architecture of healthy and diseased states. Traditional single-plex techniques, such as enzyme-linked immunosorbent assays (ELISA), are limited in their ability to provide comprehensive insights due to their focus on single analytes. In contrast, multiplexed detection methods offer the potential to simultaneously analyze multiple biomarkers, thereby providing a more holistic view of cellular and tissue states. However, existing multiplexed antibody-based techniques, including those employing fluorophores, metal markers, and DNA barcodes, face significant challenges. These challenges include the need for meticulous antibody validation, issues with spectral overlap, and the complex and time-consuming nature of sequential staining and bleaching protocols.

Fluorescent multiplexing techniques, such as multiplex immunofluorescence and tissue-based circular immunofluorescence, rely on labeling biomolecules with distinct fluorophores. While these methods can provide sensitive and specific detection, they are constrained by the limited spectral range available for fluorescence detection. Spectral overlap occurs when fluorophores emit light at similar wavelengths, making it difficult to distinguish between different targets. This overlap necessitates the use of dyes with minimal emission overlap, often restricting the number of biomarkers that can be simultaneously detected to four or five. Additionally, methods for removing or inactivating fluorophores after each round of staining, such as enzymatic digestion or chemical bleaching, can damage the sample and prolong the imaging process. As a result, detecting a large number of targets can become impractically lengthy and complex.

To address these limitations, advanced techniques like DNA barcoding have been developed, enabling higher multiplexing capabilities by avoiding spectral limitations. However, these methods typically require complex probe design and hybridization protocols, which can be cumbersome and expensive. Additionally, despite these advances, the sole reliance on antibodies remains a hurdle, as it requires rigorous validation to ensure accuracy and reproducibility. Consequently, there is a pressing need for innovative approaches that can overcome these challenges, enabling efficient and accurate multiplex detection of biomolecules in situ, without the drawbacks associated with current technologies.

The present disclosure addresses the limitations of existing multiplexed biomolecule detection methods by introducing an advanced cell painting technique, capable of being used in existing spatial biology platforms (e.g., the G4X™ Platform or ImageXpress® Confocal HT.ai system). This innovative approach combines the principles of traditional cell painting with the enhanced capabilities of cleavable linkers and sequential staining cycles. By integrating these elements, the invention enables the detection of a significantly larger number of cellular structures and biomarkers within the same sample, overcoming the spectral overlap and optical cross-talk issues inherent in conventional fluorescence-based methods.

Described herein is the use of cleavable linkers that connect targeting molecules, such as phalloidin or wheat germ agglutinin (WGA), to fluorescent dyes. These linkers can be cleaved through specific chemical, enzymatic, or photolytic reactions, allowing for the sequential removal of dyes after imaging. This cyclical process of staining, imaging, and cleaving is akin to painting a portrait or screen printing, where one color is added at a time to build a complete image. Similarly, each round of staining adds new layers of information about the cellular structures, ultimately revealing a comprehensive and detailed picture of the cell or tissue with all structures resolved. By employing four distinct fluorescent dyes in each cycle and minimizing optical cross-talk by using only two dyes per cycle when necessary, the system can achieve high-resolution, high-throughput imaging of numerous cellular components and biomarkers. By employing the compositions and methods described herein for two color sequencing with the use of the cell paints described herein enables the detection of nucleic acids and cellular components with decreased optical cross-talk challenges.

The mushroom toxin phalloidin is a small bicyclic peptide consisting of seven amino acids with a molecular weight of 789. Phalloidin binds to both large and small filamentous actin (F-actin) with high affinity, and compared to actin-specific antibodies, the non-specific binding of phalloidin is negligible, thus providing minimal background and high contrast during cellular imaging. Phalloidin-dye conjugates have been described previously, for example Capani et al Journal of Histochemistry & Cytochemistry. 2001; 49(11):1351-1361, and including a cleavable site in the linker to the fluorophore enables the conjugate to be used in the method described herein. For example, the probe may have the structure:

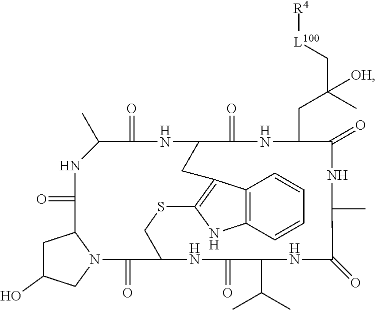

where L¹⁰⁰ is the cleavable linker and R⁴ is a fluorophore moiety.

The method also incorporates automated imaging and image analysis software, enhancing the efficiency and reproducibility of the staining and imaging process. This automation reduces manual intervention, minimizes potential errors, and facilitates large-scale studies. The resulting high-dimensional data can be integrated and analyzed to provide comprehensive profiles of cellular phenotypes, enabling detailed studies of cellular behavior, disease mechanisms, and treatment responses.

The phenotypic profile of a cell reveals the biological state of a cell. More specifically, the phenotypic profile can be used to interrogate biological perturbations because the cellular morphology is influenced by factors such as metabolism, genetic and epigenetic state of the cell, and environmental cues. In addition, it can be used to characterize healthy cells from diseased cells. Because a phenotypic profile is an aggregation of a large number of measurements, it is sensitive to deviations or changes to those features extracted using cellular paints. To create a profile of the cells, all of the features from the different organelles that are imaged and analyzed using commercially available cell imaging software (e.g., CellProfiler™) In morphological profiling, measured features include staining intensities, textural patterns, size, and shape of the labeled cellular structures, as well as correlations between stains across channels, and adjacency relationships between cells and among intracellular structures.

Existing cell paints, described in Table 2, are employed to target specific biomolecules. In current cell painting approaches, fluorescent dyes are conjugated to targeting molecules through covalent bonding, ensuring specific and stable labeling of cellular structures. The attachment process typically involves the use of chemical linkers that form a stable covalent bond between the dye and the targeting molecule. For example, phalloidin, which binds specifically to actin filaments, is covalently linked to a fluorescent dye like Alexa Fluor® 488 using a reactive group on the dye that reacts with a functional group on phalloidin. Similarly, wheat germ agglutinin (WGA), which targets the plasma membrane, is conjugated to a fluorescent dye through a linker that attaches to its glycoprotein-binding sites. This covalent linkage ensures that the dye remains firmly attached to the targeting molecule during the staining, imaging, and any subsequent washing steps, providing consistent and reliable fluorescence labeling of the intended cellular structure. Cell paints described herein are contemplated to be used to detect cellular components in tissue prepared as described supra. Alternatively, or additionally, the use of cell paints described herein are also contemplated for the in situ detection of cellular components in a tissue section prior to or following the in situ detection of nucleic acids in the same tissue section using the methods and compositions described herein.

TABLE 2
Commercially available cell paints

| Targeting | | Cell Structure |
|---|---|---|
| Molecule | Fluorescent Dye | Targeted |
| Phalloidin | Various (e.g., Alexa Fluor ® | Actin filaments |
| | 488, Alexa Fluor ® 568) | |
| Wheat Germ | Various (e.g., Alexa Fluor ® | Plasma membrane |
| Agglutinin | 488, Alexa Fluor ® 594) | |
| (WGA) | | |
| MitoTracker ® | Various (e.g., MitoTracker ® | Mitochondria |
| | Red CMXRos, MitoTracker ® | |
| | Green FM) | |
| ER-Tracker ™ | Various (e.g., ER-Tracker ™ | Endoplasmic |
| | Red, ER-Tracker ™ Green) | reticulum |
| Concanavalin A | Various (e.g., Alexa Fluor ® | Endoplasmic |
| | 350) | reticulum |
| Golgi-Tracker ™ | Various (e.g., BODIPY ® FL | Golgi apparatus |
| | C5-Ceramide) | |
| LysoTracker ® | Various (e.g., LysoTracker ® | Lysosomes |
| | Green DND-26, LysoTracker ® | |
| | Red DND-99) | |
| | CytoFix ™ Red | |
| Annexin V | Various (e.g., Annexin V Alexa | Phosphatidylserine |
| | Fluor ® 488, Annexin V FITC) | (apoptosis marker) |
| Concanavalin A | Various (e.g., Alexa Fluor ® | Cell surface |
| (ConA) | 488, Alexa Fluor ® 594) | carbohydrates |
| Transferrin | Various (e.g., Alexa Fluor ® | Transferrin |
| | 488, Alexa Fluor ® 568) | receptors |
| Lectins (e.g., | Various (e.g., Alexa Fluor ® | Specific |
| PNA, UEA-1) | 488, Alexa Fluor ® 594) | carbohydrate |
| | | structures |

The method may be useful in detecting biomolecules such as proteins and nucleic acid molecules, organelle structures such as the Golgi Apparatus, and also the cytoskeleton. The cytoskeleton is a network of different protein fibers (e.g., actin and myosin) that maintains the shape and position of the organelles within a cell. The cytoplasm, a fluid which can be rather gel-like, surrounds the nucleus, is considered an organelle.

Additional organelles detectable using the methods and compositions described herein include the Endoplasmic Reticulum (ER), which is a network of membranes that forms channels that cris-crosses the cytoplasm utilizing its tubular and vesicular structures to manufacture various molecules. The ER includes small granular structures called ribosomes useful for the synthesis of proteins. Smooth ER makes fat compounds and deactivates certain chemicals like alcohol or detected undesirable chemicals such as pesticides. Rough ER makes and modifies proteins and stores them until notified by the cell communication system to send them to organelles that require the substances. Typically, all healthy cells in humans, except erythrocytes (red blood cells) and spermatozoa, are equipped with endoplasmic reticulum. The Golgi apparatus (also referred to as a Golgi complex) consists of one or more Golgi bodies which are located close to the nucleus and consist of flattened membranes stacked atop one another like a stack of coins. The Golgi apparatus prepares proteins and lipid (fat) molecules for use in other places inside and outside the cell. Lysosomes are membrane-enclosed organelles that have an acidic interior (pH ˜4.8) and can vary in size from 0.1 to 1.2 μm. Lysosomes house various hydrolytic enzymes responsible for digesting biopolymers such as proteins, peptides, nucleic acids, carbohydrates and lipids. Ribosomes are tiny spherical organelles distributed around the cell in large numbers to synthesize cell proteins. They also create amino acid chains for protein manufacture. Ribosomes are created within the nucleus at the level of the nucleolus and then released into the cytoplasm.

The methods and compositions described herein revolutionizes cell painting techniques by introducing the use of cleavable linkers between targeting molecules and fluorescent dyes. Prior to this disclosure, the use of cleavable linkers was avoided due to concerns over stability issues, as the linkers needed to be robust enough to withstand the staining and imaging processes yet easily cleavable when desired. The invention overcomes these stability challenges by utilizing designed cleavable linkers that maintain the stability of the dye-targeting molecule complex during imaging and can be selectively cleaved using specific chemical, enzymatic, or photolytic reactions. This innovative approach enables multiple rounds of staining and imaging, significantly expanding the multiplexing capacity and allowing for the detection of a greater number of cellular structures and biomarkers within the same cell or tissue sample.

### Example 6. Applications of Two Color Sequencing and Imaging a Multiplexed Tonsil Tissue Section

To image and analyze a multiplex tonsil tissue sample using a combination of intrinsic (e.g., Hoescht 33342) and non-intrinsic ([targeting molecule]-[cleavable linker (CL)]-[fluorophore]) cell paints, employing cleavable linkers for sequential staining and imaging cycles. By spatially separating the dyes, we minimize optical cross-talk and maximize detection clarity. Cell paints described herein are contemplated to be used to detect cellular components in tissue prepared as described supra. Alternatively, or additionally, the use of cell paints described herein are also contemplated for the in situ detection of cellular components in a tissue section prior to or following the in situ detection of nucleic acids in the same tissue section using the methods and compositions described herein.

To use the cell paints described herein, the fixed and prepared tonsil tissue sample is subjected to an initial round of staining using a set of cell paints and immunostains designed to target specific cellular components. The first set includes:
Endoplasmic Reticulum: Concanavalin A (ConA)-CL-Alexa Fluor® 532 (emission: 532 nm)
Golgi Apparatus: Wheat germ agglutinin (WGA)-CL-Alexa Fluor® 594 (emission: 594 nm)
F-Actin: Phalloidin-CL-Alexa Fluor® 647 (emission: 647 nm)
Lysosomes: LysoTracker-CL-Alexa Fluor® 680 (emission: 680 nm)

Once the tissue is stained, it is imaged to capture the fluorescence signals from each dye. Following the initial imaging, the tissue sample undergoes treatment with specific cleavage reagents designed to remove the fluorescent dyes linked through cleavable linkers. The sample is then thoroughly washed to ensure complete removal of the cleaved dyes, preparing it for the next cycle of staining. In the second cycle, the tissue is stained with a new set of cell paints targeting additional structures, each conjugated with non-overlapping dyes to avoid optical cross-talk. This second set includes:
Nucleus: Hoechst 33342 (intrinsic, excitation/emission: 387/447 nm)
Nucleoli: SYTO 14 green fluorescent nucleic acid stain (intrinsic, emission: 531/593 nm)
Mitochondria: MitoTracker Deep Red (intrinsic, emission: 628/692 nm)
Transferrin Receptors: Transferrin-CL-Alexa Fluor 532 (emission: 532 nm)
Nuclear Envelope: Anti-Lamin A/C-CL-Alexa Fluor 594 (emission: 594 nm)
Cell Surface Receptors: Anti-CD3-CL-Alexa Fluor 422 (emission: 422 nm)

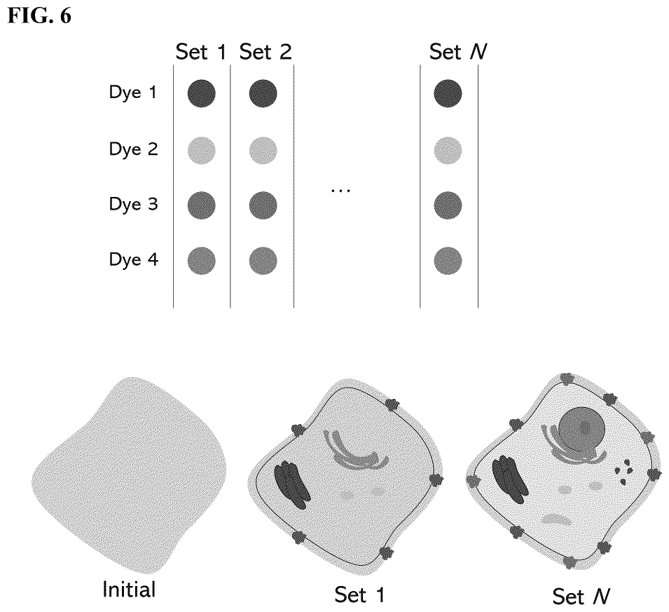

The tissue is then imaged again. After imaging, the dyes are cleaved, and the tissue is prepared for additional cycles, or detection modes, if necessary. This process of staining, imaging, and cleavage is repeated for subsequent cycles, each time introducing new cell paints to target different cellular components as illustrated in FIG. 6. Note, intrinsic stains such as Hoechst 33342 and SYTO 14, should be included in the final set so as not to interfere with detection in intervening staining cycles.

Each cycle ensures that only non-overlapping dyes are used to maintain clear separation of signals. For example, following one or more cycles using the cleavable conjugates described supra one can use traditional (i.e., non-cleavable) staining agents, such as primary antibodies (e.g., beta tubulin monoclonal antibody (ThermoFisher Scientific, 32-2600), anti-clathrin heavy chain antibody (abcam, ab21679), and anti-caveolin-1 antibody (abcam, ab2910) coupled with secondary antibody-oligonucleotide conjugates. For example, protocols for traditional immunostaining may be found in Civitci, F. et al. Protoc. Exch. doi.org/10.21203/rs.3.pex-1069/v1 (2020).

After all cycles are completed, the imaging data from each cycle are integrated using commercially available image analysis software. This software aligns the images from different cycles to create a comprehensive map of the cellular structures and biomarkers within the tonsil tissue. The data are then analyzed to quantify the expression and spatial distribution of the targeted components. By sequentially applying cell paints and utilizing cleavable linkers, this method allows for the imaging of a tonsil tissue sample, providing detailed and comprehensive visualization of various cellular components without the limitations of spectral overlap. The high-content imaging system captures high-resolution images, and the integrated data analysis offers insights into the cellular architecture and biomarker distribution within the tissue, facilitating a deeper understanding of tonsil tissue structure and function.

To facilitate the visualization of organelle and related target data commercially available software (e.g., TissueMaker®, TissueFAXS™, THUNDER™) can allow users to dynamically generate a visual interpretation of data. For example, a typical software may present a user interface with a three-dimensional representation of the cell and/or tissue. For example, the method may further include stitching. Stitching combines multiple field of view (FOV) into a single image. Stitching can be performed using a variety of techniques. For example, one approach is, for each row of FOV that together will form the combined image of the sample and each FOV within the row, determine a horizontal shift for each FOV. Once the horizontal shifting is calculated, a vertical shift is calculated for each row of FOV. The horizontal and vertical shifts can be calculated based on cross-correlation, e.g., phase correlation. With the horizontal and vertical shift for each FOV, a single combined image can be generated, and target biomolecule coordinates can be transferred to the combined image based on the horizontal and vertical shift. For the reconstruction of 3D tissues, several computational methods such as PASTE, PASTE2, SLAT, and SPACEL can be utilized. These methods and algorithms typically involve aligning detected targets between different slices and performing coordinate transformation and rotation of different slices to achieve a 3D structure composed of multiple slices.

What is claimed is:

1. A method of extending a primer, the method comprising:
(a) adding an extension solution comprising four nucleotides to a reaction vessel comprising a polymerase and the primer, wherein the primer is hybridized to a target polynucleotide, and incorporating one of four nucleotides into the primer, wherein the extension solution comprises
a first nucleotide comprising a first fluorophore moiety attached to the nucleotide via a first cleavable linker; a second nucleotide comprising a second fluorophore moiety attached to the nucleotide via a second cleavable linker; a third nucleotide comprising a third fluorophore moiety attached to the nucleotide via a third cleavable linker; a fourth nucleotide comprising a fourth fluorophore moiety attached to the nucleotide via a fourth cleavable linker, wherein the first and the second cleavable linkers are cleavable under identical conditions and the third and the fourth cleavable linkers are cleavable under different identical conditions;
(b) exciting a fluorophore, wherein exciting comprises:
(i) directing a first excitation light and second excitation light at the reaction vessel;
(ii) adding a first cleaving agent into the reaction vessel; followed by
(iii) adding a second cleaving agent into the reaction vessel.

2. The method of claim 1, further comprising detecting the incorporated nucleotide, wherein detecting comprises detecting an emission light after step (i).

3. The method of claim 2, further comprising determining the identity of the incorporated nucleotide based on the detection of the emission light.

4. The method of claim 2, further comprising determining the identity of the incorporated nucleotide based on the detection of the emission light after step (i) and detecting the absence of the emission light after step (ii).

5. The method of claim 1, further comprising detecting the incorporated nucleotide, wherein detecting comprises detecting a first emission light after step (i) and detecting a second emission light after step (ii).

6. The method of claim 5, further comprising determining the identity of the incorporated nucleotide based on the detection of the first emission light and the second emission light.

7. The method of claim 1, wherein the extension solution comprises
an adenine nucleotide comprising a first fluorophore moiety attached to the nucleotide via a first cleavable linker; a guanine nucleotide comprising a second fluorophore moiety attached to the nucleotide via a second cleavable linker; a cytosine nucleotide comprising a third fluorophore moiety attached to the nucleotide via a third cleavable linker; a thymine nucleotide comprising a fourth fluorophore moiety attached to the nucleotide via a fourth cleavable linker.

8. The method of claim 1, wherein the extension solution comprises
an adenine nucleotide comprising a first fluorophore moiety attached to the nucleotide via a first cleavable linker; a cytosine nucleotide comprising a second fluorophore moiety attached to the nucleotide via a second cleavable linker; a guanine nucleotide comprising a third fluorophore moiety attached to the nucleotide via a third cleavable linker; a thymine nucleotide comprising a fourth fluorophore moiety attached to the nucleotide via a fourth cleavable linker.

9. The method of claim 1, wherein
the first cleavable linker and the second cleavable linker are selected from the group: an enzyme-cleavable linker, a photocleavable linker, an acid-cleavable linker, a base-cleavable linker, an oxidant-cleavable linker, a reductant-cleavable linker, or a fluoride-cleavable linker; and
the third cleavable linker and the fourth cleavable linker are selected from the group: an enzyme-cleavable linker, a photocleavable linker, an acid-cleavable linker, a base-cleavable linker, an oxidant-cleavable linker, a reductant-cleavable linker, or a fluoride-cleavable linker.

10. The method of claim 1, wherein
the first cleavable linker and the second cleavable linker comprise a disulfide moiety, a dialkylketal moiety, an allyl moiety, an azide moiety, a hydrazine moiety, a cyanoethyl moiety, or a nitrobenzyl moiety.

11. The method of claim 1, wherein the first nucleotide, the second nucleotide, the third nucleotide, and the fourth nucleotide each independently comprise a reversible terminator.

12. The method of claim 1, wherein the first nucleotide, the second nucleotide, the third nucleotide, and the fourth nucleotide each independently comprise the formula: 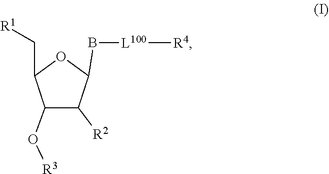
wherein
B is a divalent nucleobase;
R¹ is independently a polyphosphate moiety, monophosphate moiety, or nucleic acid moiety;
R² is hydrogen or —OH;
R³ is a reversible terminator;
L¹⁰⁰ is a cleavable linker; and
R⁴ is a fluorophore moiety.

13. The method of claim 1, wherein the first nucleotide, the second nucleotide, the third nucleotide, and the fourth nucleotide each independently comprise the formula: 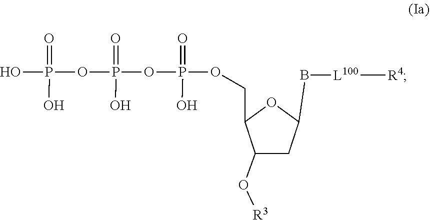
Wherein,
B is a divalent nucleobase;
R³ is a reversible terminator;
L¹⁰⁰ is a cleavable linker; and
R⁴ is a fluorophore moiety.

14. The method of claim 12, wherein B is:

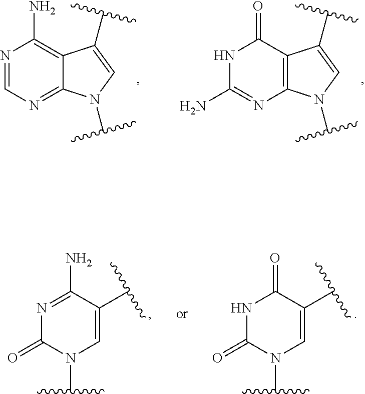 [SMILES: *c1cn(*)c2ncnc(N)c12 ; *c1cn(*)c2nc(N)[nH]c(=O)c12 ; *c1cn(*)c(=O)nc1N ; *c1cn(*)c(=O)[nH]c1=O]

15. The method of claim 1, wherein the first nucleotide, the second nucleotide, the third nucleotide, and the fourth nucleotide are selected from the following group: 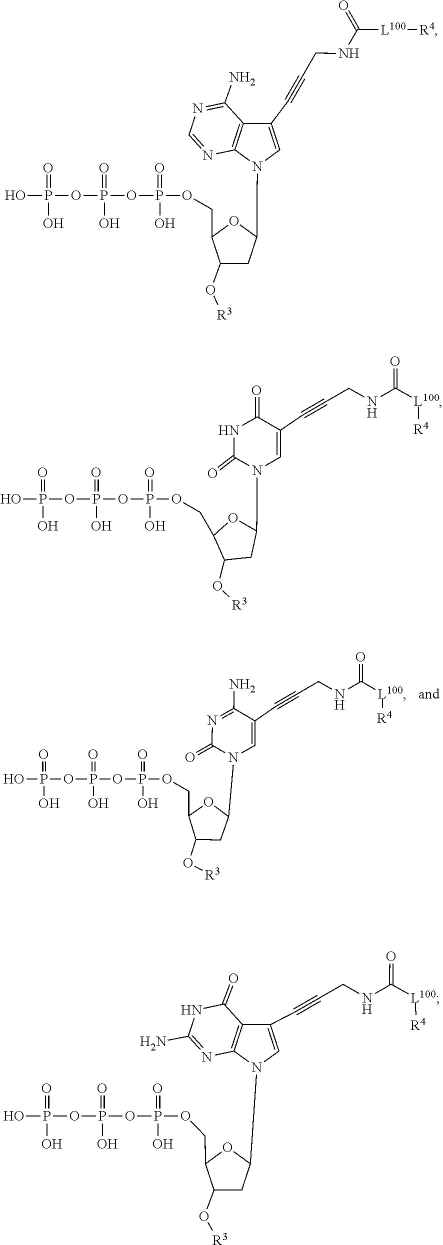
wherein
R³ is a reversible terminator;
L¹⁰⁰ is a cleavable linker; and
R⁴ is a fluorophore moiety.

16. The method of claim 12, wherein L¹⁰⁰ is a divalent linker comprising 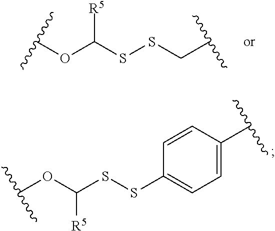
wherein,
R⁵ is independently substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

17. The method of claim 12, wherein L¹⁰⁰ is a divalent linker comprising 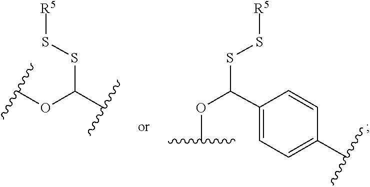
wherein R⁵ is unsubstituted C₁-C₄ alkyl.

18. The method of claim 12, wherein L¹⁰⁰ is -L¹⁰¹-L¹⁰²-L¹⁰³-L¹⁰⁴-L¹⁰⁵-; wherein,
L¹⁰¹, L¹⁰², L¹⁰³, L¹⁰⁴, and L¹⁰⁵ are independently a bond, —NH—, —O—, —C(O)—,
—C(O)NH—, —NHC(O)—, —NHC(O)NH—, —C(O)O—, —OC(O)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

19. A method of sequencing a template polynucleotide, said method comprising:
(a) contacting a first primer hybridized to the template polynucleotide with four nucleotides and incorporating with a polymerase one of said four nucleotides into the first primer, wherein
two of the four nucleotides comprise a first fluorophore moiety attached to each nucleotide via a first cleavable linker and two of the four nucleotides comprise a second fluorophore moiety attached to each nucleotide via a second cleavable linker, wherein the first fluorophore moiety generates a first emission light and the second fluorophore moiety generates a second emission light;
(b) determining the identity of the incorporated nucleotide by:
(i) detecting the first emission light; cleaving the first cleavable linker; and detecting the absence of the first emission light;
(ii) detecting the first emission light; cleaving the first cleavable linker; and detecting the first emission light again;
(iii) detecting the second emission light; cleaving the first cleavable linker; and detecting the absence of the first emission light; or
(iv) detecting the second emission light; cleaving the first cleavable linker; and detecting the second emission light;
(c) repeating steps (a) and (b), thereby sequencing a template polynucleotide.

20. A composition comprising:
a first nucleotide comprising a first fluorophore moiety attached to the nucleotide via a first cleavable linker;
a second nucleotide comprising a second fluorophore moiety attached to the nucleotide via a second cleavable linker;
a third nucleotide comprising a third fluorophore moiety attached to the nucleotide via a third cleavable linker;
a fourth nucleotide comprising a fourth fluorophore moiety attached to the nucleotide via a fourth cleavable linker;
wherein the first and the second cleavable linkers are cleavable under identical conditions and the third and the fourth cleavable linkers are cleavable under identical conditions.
